(19) United States
(12) Patent Application Publication
(10) Pub. No.: US 2025/0340643 A1
(43) Pub. Date: Nov. 6, 2025

(54) Anti-CTLA Antibody Compositions and Related Methods

(71) Applicant: Astrazeneca AB

(72) Inventors: Xia LUO, Gaithersburg, MD (US); Limin QU, Gaithersburg, MD (US); Chris AFDAHL, Gaithersburg, MD (US); Jessica Lynn PRENTICE, Gaithersburg, MD (US)

(21) Appl. No.: 19/126,456

(22) PCT Filed: Nov. 3, 2023
(86) PCT No.: PCT/EP2023/080627

§ 371 Date: May 1, 2025

Related U.S. Application Data
(60) Provisional application No. 63/382,398, filed on Nov. 4, 2022.

(51) Int. Cl. C07K 16/28; A61K 9/00; A61K 47/18; A61K 47/22; A61K 47/26

(52) U.S. Cl. CPC C07K 16/2818; A61K 9/0019; A61K 47/183; A61K 47/22; A61K 47/26; C07K 2317/40; C07K 2317/53; C07K 2317/565; C07K 2317/76; C07K 2317/92; C07K 2317/94

(57) ABSTRACT

The present disclosure provides compositions comprising anti-CTLA-4 antibodies and related methods for treating cancer and other disorders responsive to anti-CTLA-4 antagonism.

### FIELD OF THE DISCLOSURE

The present disclosure relates to compositions comprising anti-CTLA antibodies and related methods for treating cancer.

### SEQUENCE LISTING STATEMENT

A computer readable form of the Sequence Listing is filed with this application by electronic submission and is incorporated into this application by reference in its entirety. The Sequence Listing is contained in the file created on Oct. 20, 2022 having the file name “22-1658-US-PRO.xml” and is 19 kb in size.

### BACKGROUND

Cancer continues to be a major global health burden. In the United States, it is the second most common cause of death after heart disease, accounting for nearly 1 in every 4 deaths. The 5-year survival rate for all cancers diagnosed between 1999 and 2006 is 68%, which is 18% higher than the rate reported between 1975 and 1977, likely reflecting progress in diagnosing certain cancers earlier and improvements in treatment.

About 12.7 million cancer cases and 7.6 million cancer deaths are estimated to have occurred in 2008 worldwide. Most often these cancers are diagnosed at an advanced or metastatic stage where the life expectancy is very poor. Despite recent advances in chemotherapeutics and in the understanding of the molecular biology of cancer, there has been limited progress in the therapeutic options for advanced and metastatic disease. The poor prognosis reflects the limited efficacy of the treatment options available, highlighting the need for the development of newer therapeutic options.

The role of the immune system, in particular T cell-mediated cytotoxicity, in tumor and infection control is well recognized. There is mounting evidence that T cells control tumor growth and survival in cancer patients, both in early and late stages of the disease. However, tumor-specific T-cell responses are difficult to mount and sustain in cancer patients.

CTLA4 is expressed on activated T cells and serves as a co-inhibitor to keep T cell responses in check following CD28-mediated T cell activation. CTLA4 is believed to regulate the amplitude of the early activation of naïve and memory T cells following TCR engagement and to be part of a central inhibitory pathway that affects both antitumor immunity and autoimmunity. CTLA4 is expressed exclusively on T cells, and the expression of its ligands CD80 (B7.1) and CD86 (B7.2), is largely restricted to antigen-presenting cells, T cells, and other immune mediating cells. Antagonistic anti-CTLA4 antibodies that block the CTLA4 signalling pathway have been reported to enhance T cell activation in cancer and infection conditions and disorders.

Tremelimumab is a humanized immunoglobulin (Ig) G2 monoclonal antibody directed against the human T-cell receptor protein cytotoxic T-lymphocyte-associated protein 4 (CTLA4), with potential immune checkpoint inhibitory and antineoplastic activities.

### SUMMARY

The disclosure generally relates to compositions comprising anti-CTLA antibodies and related methods for treating cancer.

In one aspect, the disclosure herein provides a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; and one or more deamidated variants of the anti-CTLA-4 antibody, wherein the composition comprises ≤45%, ≤40%, ≤35%, ≤30%, ≤20%, ≤15%, ≤10%, ≤5%, ≤4%, or ≤3% of the deamidated variants.

In another aspect, the disclosure herein provides a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; and one or more oxidized variants of the anti-CTLA antibody, wherein the composition comprises ≤35%, ≤30%, ≤20%, ≤15%, ≤10%, ≤5%, ≤4%, or ≤3% of the oxidized variants.

In another aspect, the disclosure herein provides a composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; and one or more aggregated variants of the anti-CTLA-4 antibody, wherein the composition comprises ≤26%, ≤25%, ≤20%, ≤10%, ≤5%, ≤4%, ≤3%, ≤2%, or ≤1% of the aggregated variants.

In another aspect, the disclosure herein provides a composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; and a fragmented variant of the CTLA-4 antibody, wherein the composition comprises ≤10%, ≤5%, ≤4%, ≤3%, ≤2%, or ≤1% fragmented variant.

In another aspect, the disclosure herein provides a composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; and up to 100% heavy chain N-terminal pyro-glutamate variant and/or up to 100% heavy chain C-terminal lysine cleaved variant of the CTLA-4 antibody.

In another aspect, the disclosure herein provides a composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; further comprising ≤34 ng/mg of host cell protein (HCP).

In another aspect, the disclosure herein provides a composition comprising an anti-CTLA-4 antibody having a heavy chain comprising the amino acid sequence of SEQ ID NO: 10 and a light chain comprising the amino acid sequence of SEQ ID NO: 9, wherein the composition comprises: (i)≤35%, ≤30%, ≤20%, ≤15%, ≤10%, ≤5%, ≤4%, or ≤3% oxidized variant of the anti-CTLA-4 antibody; (ii)≤26%, ≤25%, ≤20%, ≤10%, ≤5%, ≤4%, ≤3%, ≤2%, or ≤1% aggregated variant of the anti-CTLA-4 antibody; (iii)≤45%, ≤40%, ≤35%, 30%, ≤20%, ≤15%, ≤10%, ≤5%, ≤4%, or ≤3% deamidated variant of the anti-CTLA-4 antibody; (iv)≤10%, ≤5%, ≤4%, ≤3%, ≤2%, or ≤1% fragmented variant of the anti-CTLA-4 antibody.

In another aspect, the disclosure herein provides a composition comprising an anti-CTLA-4 antibody having a heavy chain comprising the amino acid sequence of SEQ ID NO: 10 and a light chain comprising the amino acid sequence of SEQ ID NO: 9, wherein the composition further comprises: (i)≤30% of a variant oxidized at Heavy Chain Trp-52 of the anti-CTLA-4 antibody; (ii)≤35% of a variant deamidated at Heavy Chain Met-256 and/or Heavy Chain Met-432 of the anti-CTLA-4 antibody; (iii)≤34 ng/mg Host Cell protein; (iv) ≤4% aggregated variant of the anti-CTLA-4 antibody; and/or (iv)≤10% fragmented variant of the anti-CTLA-4 antibody.

These and other features and advantages of the present disclosure will be more fully understood from the following detailed description taken together with the accompanying claims. It is noted that the scope of the claims is defined by the recitations therein and not by the specific discussion of features and advantages set forth in the present description

### BRIEF DESCRIPTION OF THE DRAWINGS

FIG. 1 shows IEC profiles of fraction B, D, E.

FIG. 2 shows cIEF analysis of IEC fractions.

FIG. 3 shows the effect of LC Asn-30 deamidation on potency.

FIG. 4 shows fragmentation sites of tremelimumab

### DETAILED DESCRIPTION

The present disclosure relates to compositions comprising anti-CTLA antibodies and related methods for treating cancer.

### 1. General Definitions

The term “antibody” as used herein in the broadest sense to refer to molecules with an immunoglobulin-like domain (e.g., IgG, IgM, IgA, IgD, or IgE) and includes monoclonal, recombinant, polyclonal, chimeric, human, and humanized molecules of this type.

The term, full, whole or intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallizable fragment. The Fab fragment is composed of the variable region at the amino-terminus, variable heavy (VH) or variable light (VL), and the constant region at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences which are called m, a, g, e and d respectively, each heavy chain can pair with either a K or 1 light chain. The majority of antibodies in the serum belong to the IgG class, there are four isotypes of human IgG, IgG1, IgG2, IgG3 and IgG4, the sequences of which differ mainly in their hinge region.

Fully human antibodies can be obtained using a variety of methods, for example using yeast-based libraries or transgenic animals (e.g., mice) which are capable of producing repertoires of human antibodies. Yeast presenting human antibodies on their surface which bind to an antigen of interest can be selected using FACS (Fluorescence-Activated Cell Sorting) based methods or by capture on beads using labelled antigens. Transgenic animals that have been modified to express human immunoglobulin genes can be immunized with an antigen of interest and antigen-specific human antibodies isolated using B-cell sorting techniques. Human antibodies produced using these techniques can then be characterized for desired properties such as affinity, developability and selectivity.

Monoclonal antibodies may be produced by a eukaryotic cell clone or a prokaryotic cell clone expressing an antibody. Monoclonal antibodies may also be produced by a eukaryotic cell line which can recombinantly express the heavy chain and light chain of the antibody by virtue of having nucleic acid sequences encoding these introduced into the cell. Exemplary methods to produce antibodies from different eukaryotic cell lines such as Chinese Hamster Ovary cells, hybridomas or immortalized antibody cells derived from an animal (e.g., human) are well known to those skilled in the art.

The antibody may be derived, for example, from either rat, mouse, primate (e.g., cynomolgus, Old World monkey or Great Ape), human or other sources such as nucleic acids generated using molecular biology techniques known to those skilled in the art which encode an antibody molecule.

The antibody may be either a fully human, a humanized, or a chimeric antibody. In one embodiment, the antibody is a humanized antibody. In one embodiment, the antibody is a monoclonal antibody.

The antibody may comprise one or more modifications including, for example, a mutated constant domain such that the antibody has enhanced effector functions/ADCC and/or complement activation.

The antibody may comprise two immunoglobulin (Ig) heavy chains (“HC”) and two Ig light chains (“LC”). The basic antibody structural unit may comprise, for example, a tetramer of subunits. Each tetramer may include two pairs of polypeptide chains, each pair having one “light” (about 25 kDa) and one “heavy” chain (about 50-70 kDa). The amino-terminal portion of each chain may include a variable region of about 100 to 110 or more amino acids primarily responsible for antigen recognition. This variable region may initially be expressed linked to a cleavable signal peptide.

The variable region without the signal peptide may be referred to as a mature variable region. Thus, in one example, a light chain mature variable region may comprise a light chain variable region without the light chain signal peptide. The carboxy-terminal portion of each chain may define a constant region. In one embodiment, the antibody of the compositions described herein is a full-length antibody.

The terms “VH” and “VL” are used herein to refer to the heavy chain variable region and light chain variable region respectively of an antibody.

The mature variable regions of each light/heavy chain pair may form the antibody binding site (also referred to as the antigen binding site). “Antigen binding site” refers to a site on an antibody which is capable of specifically binding to an antigen, this may be a single variable domain, or it may be paired VH/VL domains as can be found on a standard antibody. Thus, an intact antibody may have, for example, two binding sites. Except in bifunctional or bispecific antibodies, the two binding sites can be the same. The chains all may exhibit the same general structure of relatively conserved framework regions (FR) joined by three hypervariable regions, also called complementarity determining regions or “CDRs”.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs.

The CDRs from the two chains of each pair may be aligned by the framework regions, enabling binding to a specific epitope. Thus, in one example, from N-terminal to C-terminal, both light and heavy chains comprise the domains FR1, CDR1, FR2, CDR2, FR3, CDR3 and FR4.

Acceptable heavy chain variable region and light chain variable region framework 1 framework 2 and framework 3 regions are readily recognized by those of ordinary skill in the art. Acceptable heavy chain constant regions (including hinge regions) and light chain constant regions are readily recognized by those of ordinary skill in the art as well. Acceptable antibody isotypes are similarly readily recognized by those of ordinary skill in the art.

“CDRs” are defined as the complementarity determining region amino acid sequences of an antibody. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, the terms “CDR,” “CDRL1,” “CDRL2,” “CDRL3,”

“CDRH1,” “CDRH2,” “CDRH3” follow the Kabat numbering convention. The amino acid residues in the variable region sequences and full length antibody sequences are numbered sequentially to denote any antibody variant position or post-translational modification variant position

The term “antigen-binding fragment” refers to a portion of an intact antibody and/or refers to the antigenic determining variable domains of an intact antibody. It is known that the antigen-binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

The terms “variant,” “antibody variant,” “CDR variant” and “post-translational modification variant” refer to a variant antibody sequence wherein at least one amino acid sequence has been changed with respect to the antibody sequence, for example via a post translational modification, a chemical change or a sequence change via at least one deletion, substitution or addition. Some post-translational modifications result in a chemical change which does not change the sequence (e.g., Met and oxidized Met; or Asp and isomarized/iso-Asp; or aggregation) while others result in a sequence change such as the conversion of one amino acid residue into another (e.g., Asn conversion to Asp via deamidation; or lysine deletion). Further post-translational modification variants are described below. A variant antibody sequence which comprises a sequence change may be the result of a designed sequence change or a post-translational modification.

The amino acid replacement or substitution can be conservative, semiconservative, or non-conservative. Amino acids are broadly grouped as “aromatic” or “aliphatic”. An aromatic amino acid includes an aromatic ring (e.g., histidine, phenylalanine, tyrosine, and tryptophan). Non-aromatic amino acids are broadly grouped as “aliphatic”.

In one embodiment, substitutions are conservative substitutions. It is well recognized in the art that certain amino acid substitutions are regarded as being “conservative”. Amino acids may be further divided into groups based on common side-chain properties and substitutions within groups that maintain all or substantially all of the binding affinity of the antibody are regarded as conservative substitutions.

For example, groups of amino acids include: amino acid residues with hydrophobic side chains such as methionine, alanine, valine, leucine and isoleucine; amino acids with neutral, hydrophilic side chains such as cysteine, serine and threonine; amino acids with acidic side chains such as aspartic acid and glutamic acid; amino acids with basic side chains such as asparagine, glutamine, histidine, lysine and arginine; amino acids with residues that influence chain orientation such as glycine and proline; and amino acids with aromatic side chains such as tryptophan, tyrosine and phenylalanine. The antibodies disclosed herein can comprise such “conservative” amino acid substitutions. In an alternative embodiment, an antibody variant comprises at least one substitution whilst retaining the canonical of the antibody.

“Semi-conservative mutations” include amino acid substitutions of amino acids within the broad group (i.e., aromatic or aliphatic), but not within the same side chain sub-group. For example, the substitution of aspartic acid for asparagine, or asparagine for lysine, involves amino acids within the same group (i.e., aliphatic), but different subgroups. “Non-conservative mutations” involve amino acid substitutions between different groups, for example, lysine for tryptophan, or phenylalanine for serine, etc.

In one embodiment, an antibody variant is an antibody that is at least about 80%, about 85%, about 90%, about 95%, about 96%, about 97%, about 98% or about 99% identical to (i.e., has sequence identity to) the antibody primary sequence. In another embodiment, an antibody variant comprises an antibody comprising a heavy chain amino acid sequence that is at least about 80%, about 85%, about 90%, about 95%, about 96%, about 97%, about 98% or about 99% identical to the amino acid sequence of SEQ ID NO: 10 and/or a light chain amino acid sequence that is at least about 80%, about 85%, about 90%, about 95%, about 96%, about 97%, about 980% or about 99% identical to the amino acid sequence of SEQ ID NO: 9.

“Percent identity” between a query nucleic acid sequence and a subject nucleic acid sequence is the “Identities” value, expressed as a percentage, that is calculated using a suitable algorithm or software, such as BLASTN, FASTA, DNASTAR Lasergene, GeneDoc, Bioedit, EMBOSS needle or EMBOSS infoalign, over the entire length of the query sequence after a pair-wise global sequence alignment has been performed using a suitable algorithm or software, such as BLASTN, FASTA, ClustalW, MUSCLE, MAFFT, EMBOSS Needle, T-Coffee, and DNASTAR Lasergene.

Importantly, a query sequence may be described by a nucleic acid sequence identified in one or more claims herein.

“Percent identity” between a query amino acid sequence and a subject amino acid sequence is the “Identities” value, expressed as a percentage, that is calculated using a suitable algorithm or software, such as BLASTP, FASTA, DNASTAR Lasergene, GeneDoc, Bioedit, EMBOSS needle or EMBOSS infoalign, over the entire length of the query sequence after a pair-wise global sequence alignment has been performed using a suitable algorithm/software such as BLASTP, FASTA, ClustalW, MUSCLE, MAFFT, EMBOSS Needle, T-Coffee, and DNASTAR Lasergene.

Importantly, a query sequence may be described by an amino acid sequence identified in one or more claims herein.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDRs. Alternatively, the % identity may exclude one or more or all of the CDRs, for example all of the CDRs are 100% identical to the subject sequence and the % identity variation is in the remaining portion of the query sequence, for example, the framework sequence, so that the CDR sequences are fixed and intact.

The amino acid sequences which may be useful, and included, in compositions and related methods of the disclosure may have between about 85% to about 100%, about 90% to about 100%, about 95% to about 100%, about 91%, about 92%, about 93%, about 94%, about 95%, about 96%, about 97%, about 98%, about 99% and about 100% identity to the amino acid sequences identified in the disclosure (e.g., to an antibody heavy chain or antibody light chain). In the disclosure, percent identity between the amino acid sequences described may include any discrete subrange of the percent identity ranges recited above (e.g., any range of integer values within a particular range or discrete sub-values within a particular range).

The term “specifically binds” or “binds specifically,” as used herein in relation to antibodies, means that the antibody binds to a target antigen as well as a discrete domain, or discrete amino acid sequence, within a target antigen with no or insignificant binding to other (for example, unrelated) proteins. This term, however, does not exclude the fact that the antibody may also be cross-reactive with closely related molecules (for example, those with a high degree of sequence identity or from another genera or species). The antibodies described herein may bind to human CTLA-4 with at least 2, 5, 10, 50, 100, or 1000-fold greater affinity than they bind to closely related molecules.

Affinity, also referred to as “binding affinity,” is the strength of binding at a single interaction site, i.e., of one molecule, e.g., an antibody, to another molecule, e.g., its target antigen, at a single binding site. The binding affinity of an antibody to its target may be determined by equilibrium methods (e.g., enzyme-linked immunoabsorbent assay (ELISA) or radioimmunoassay (RIA)), or kinetics (e.g., surface plasmon resonanace analysis using a BIACORE or similar instrument).

The binding affinity (KD) of the antibody-target antigen interaction may be, for example, from about 1 picomolar (pM) to about 100 micromolar (μM) (e.g., from about 1 picomolar (pM) to about 1 nanomolar (nM), from about 1 nM to about 1 micromolar (μM), or from about 1 pM to about 100 pM). In some embodiments, the anti-CTLA-4 antibody can bind to a CTLA-4 protein with a KD less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In some embodiments, the anti-CTLA-4 antibody can bind to CTLA-4 with a KD less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values).

Alternatively, the KD may be between 1 pM and 1000 pM, such as between 10 pM and 800 pM, for example about 700 pM. The binding affinity of the antibody is determined by the association constant (Ka) and the dissociation constant (Kd) (KD=Kd/Ka). The binding affinity may be measured by BIACORE (surface plasmon resonance), for example, by capture of the test antibody onto a protein-A coated sensor surface and flowing target antigen over this surface. Alternatively, the binding affinity can be measured by FORTEBIO, for example, with the test antibody receptor captured onto a protein-A coated needle and flowing target antigen over this surface.

The K may be 1×103 Ms 1 or less. The Kd may be between 1×105 Ms 1 and 1 ×103 Ms-1; or between 1×104 Ms-1 and 1×103 Ms-1. A slow Kd may result in a slow dissociation of the antibody-target antigen complex and improved neutralization of the target antigen.

The term “specific antigen binding activity” as used herein means antigen binding activity as measured by Surface Plasmon Resonance (SPR). CTLA-4 specific binding activity may be determined by SPR using a BIACORE instrument, for example performed in the binding mode. It is binding activity divided by total protein (e.g., tremelimumab) content in a sample. The term “FcRn binding activity” as used herein means Neonatal Fc (FcRn) Receptor binding activity as measured by Surface Plasmon Resonance (SPR). FcRn binding may be determined using a BIACORE instrument. It is binding activity to the FcRn receptor, divided by the total protein concentration of the sample.

The SPR method for specific antigen binding and FcRn binding uses a reference standard of tremelimumab. The tremelimumab reference standard can be used in assays to obtain system suitability and sample comparability data, to ensure methods are performing appropriately. The reference standard can allow the establishment of a calibration curve and concentrations of the samples are interpolated from the curve.

Potency is herein defined as the inhibitory activity of the anti-CTLA-4 antibody or the composition as described herein to inhibit ligand (CTLA-4) binding to CTLA-4. This may be measured by specific binding to the antigen CTLA-4, by a potency assay (e.g., IL-2 reporter assay), or by a potency reporter gene bioassay. The potency assay may be a cell-based competitive binding assay, which measures the dose-dependent ability of the antibody or the composition to inhibit CTLA-4 ligand binding to B7 ligands (CD80, CD86). Results can be reported as percent potency relative to reference material (e.g., control sample).

The terms “peptide,” “polypeptide,” “protein,” and “peptide chain” each refer to a molecule comprising two or more amino acid residues. A peptide may be monomeric or polymeric.

References to “about” as used herein when referring to a measurable value such as an amount, a temporal duration, and the like, is meant to encompass variations of ±20% or ±10%, including ±5%, ±1%, and ±0.1% from the specified value, as such variations are appropriate to perform the disclosed methods.

### 2. Anti-CTLA-4 Antibodies

In some embodiments, the anti-CTLA-4 antibody or antigen-binding fragment thereof is tremelimumab. Tremelimumab and antigen-binding fragments thereof for use in the methods compositions, and combinations provided herein comprises a heavy chain and a light chain or a heavy chain variable region and a light chain variable region. In some embodiments, tremelimumab or antigen-binding fragment thereof for use in the methods compositions, and combinations provided herein comprises a light chain variable region comprising the amino acid sequence of SEQ ID NO: 7 and a heavy chain variable region comprising the amino acid sequence of SEQ ID NO: 8. In some embodiments, tremelimumab or antigen-binding fragment thereof for use in the methods compositions, and combinations provided herein comprises a heavy chain variable region and a light chain variable region, wherein the heavy chain variable region comprises the Kabat-defined CDR1, CDR2, and CDR3 sequences of SEQ ID NOs: 4-6, and wherein the light chain variable region comprises the Kabat-defined CDR1, CDR2, and CDR3 sequences of SEQ ID NOs: 1-3. Those of ordinary skill in the art would easily be able to identify Chothia-defined, Abm-defined, or other CDR definitions known to those of ordinary skill in the art. In some embodiments, tremelimumab or antigen-binding fragment thereof for use in the methods compositions, and combinations provided herein comprises or the variable heavy chain and variable light chain CDR sequences of the 11.2.1 antibody as disclosed in U.S. Pat. No. 6,682,736, which is incorporated herein by reference in its entirety.

Tremelimumab light chain (LC) variable
region:
(SEQ ID NO: 7)
PSSLSASVGDRVTITCRASQSINSYLDWYQQKPGKAPKLLIYAASSLQSG
VPSRFSGSGSGTDFTLTISSLQPEDFATYYCQQYYSTPFTFGPGTKVEIK
RTVAAPSVFIFPPSDEQLKSGTASVVCLLNNFYPREAKV
Tremelimumab light chain (LC) variable
region (deamidated LCDR1):
(SEQ ID NO: 13)
PSSLSASVGDRVTITCRASQSIDSYLDWYQQKPGKAPKLLIYAASSLQSG
VPSRFSGSGSGTDFTLTISSLQPEDFATYYCQQYYSTPFTFGPGTKVEIK
RTVAAPSVFIFPPSDEQLKSGTASVVCLLNNFYPREAKV
Tremelimumab heavy chain (HC) variable
region:
(SEQ ID NO: 8)
GVVQPGRSLRLSCAASGFTFSSYGMHWVRQAPGKGLEWVAVIWYDGSNKY
YADSVKGRFTISRDNSKNTLYLQMNSLRAEDTAVYYCARDPRGATLYYYY
YGMDVWGQGTTVTVSSASTKGPSVFPLAPCSRSTSESTAALGCLVKDYFP
EPVTVSWNSGALTSGVH
Tremelimumab heavy chain (HC) variable
region (oxidized HC-CDR-2):
(SEQ ID NO: 14)
GVVQPGRSLRLSCAASGFTFSSYGMHWVRQAPGKGLEWVAVIXYDGSNKY
YADSVKGRFTISRDNSKNTLYLQMNSLRAEDTAVYYCARDPRGATLYYYY
YGMDVWGQGTTVTVSSASTKGPSVFPLAPCSRSTSESTAALGCLVKDYFP
EPVTVSWNSGALTSGVH (X is oxidized methionine)
Tremelimumab heavy chain CDRs
HC-CDR1:
(SEQ ID NO: 4)
GFTFSSYGMH
HC-CDR2:
(SEQ ID NO: 5)
VIWYDGSNKYYADSV
HC-CDR3:
(SEQ ID NO: 6)
DPRGATLYYYYYGMDV
HC-CDR2 (oxidized):
(SEQ ID NO: 12)
VIXYDGSNKYYADSV (X is oxidized tryptophan)
Tremelimumab light chain CDR1s
LC-CDR1:
(SEQ ID NO: 1)
RASQSINSYLD
LC-CDR2:
(SEQ ID NO: 2)
AASSLQS
LC-CDR3:
(SEQ ID NO: 3)
QQYYSTPFT
LC-CDR1 (deamidated):
(SEQ ID NO: 11)
RASQSIDSYLD
Tremelimumab Full light chain
(SEQ ID NO: 9)
DIQMTQSPSSLSASVGDRVTITCRASQSINSYLDWYQQKPGKAPKLLIYA
ASSLQSGVPSRFSGSGSGTDFTLTISSLQPEDFATYYCQQYYSTPFTFGP
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Tremelimumab Full heavy chain
(SEQ ID NO: 10)
QVQLVESGGGVVQPGRSLRLSCAASGFTFSSYGMHWVRQAPGKGLEWVAV
IWYDGSNKYYADSVKGRFTISRDNSKNTLYLQMNSLRAEDTAVYYCARDP
RGATLYYYYYGMDVWGQGTTVTVSSASTKGPSVFPLAPCSRSTSESTAAL
GCLVKDYFPEPVTVSWNSGALTSGVHTFPAVLQSSGLYSLSSVVTVPSSN
FGTQTYTCNVDHKPSNTKVDKTVERKCCVECPPCPAPPVAGPSVFLFPPK
PKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREP
QVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPG
K.
Tremelimumab Full heavy chain
(SEQ ID NO: 15)
QVQLVESGGGVVQPGRSLRLSCAASGFTFSSYGMHWVRQAPGKGLEWVAV
IX₁YDGSNKYYADSVKGRFTISRDNSKNTLYLQMNSLRAEDTAVYYCARD
PRGATLYYYYYGMDVWGQGTTVTVSSASTKGPSVFPLAPCSRSTSESTAA
LGCLVKDYFPEPVTVSWNSGALTSGVHTFPAVLQSSGLYSLSSVVTVPSS
NFGTQTYTCNVDHKPSNTKVDKTVERKCCVECPPCPAPPVAGPSVFLFPP
KPKDTLX₂ISRTPEVTCVVVDVSHEDPEVQFNWYVDGVEVHNAKTKPREE
QFNSTFRVVSVLTVVHQDWLNGKEYKCKVSNKGLPAPIEKTISKTKGQPR
EPQVYTLPPSREEX₂TKNQVSLTCLVKGFYPSDIAVEWESX₃GQPEX₃NY
KTTPPX₂LDSDGSFFLYSKLTVDKSRWQQGNVFSCSVX₂HEALHNHYTQK
SLSLSPGK (wherein X₃ is asparagine which may be
deamidated, X₂ is methionine or oxidized
methionine and X₁ is tryptophan or oxidized
tryptophan).

The compositions of the disclosure may comprise an anti-CTLA-4 antibody comprising one or more CDRs described herein, or one or both of the heavy or light chain variable regions described herein, or one or both of the heavy or light chains described herein.

In one aspect, the composition comprises an antibody having a heavy chain sequence comprising a CDRH1 comprising the amino acid sequence of SEQ ID NO: 4, a CDRH2 comprising the amino acid sequence of SEQ ID NO: 5, and a CDRH3 comprising the amino acid sequence of SEQ ID NO: 6, and a light chain sequence comprising a CDRL1 comprising the amino acid sequence of SEQ ID NO: 1, a CDRL2 comprising the amino acid sequence of SEQ ID NO: 2, and a CDRL3 comprising the amino acid sequence of SEQ ID NO: 3.

In one embodiment, the anti-CTLA-4 antibody comprises a heavy chain variable region CDR2 (“CDRH2”) comprising an amino acid sequence with one or two amino acid variation(s) (“CDR variant”) to the amino acid sequence set forth in SEQ ID NO: 5

In one embodiment, the anti-CTLA-4 antibody comprises a heavy chain variable region CDR2 (“CDRH2”) comprising an amino acid sequence with five or less, such as four or less, three or less, two or less, or one amino acid variation(s) (“CDR variant”) to the amino acid sequence set forth in SEQ ID NO: 5. In a further embodiment, the CDRH1 comprises an amino acid sequence with one or two amino acid variation(s) to the amino acid sequence set forth in SEQ ID NO: 4.

In one embodiment, the anti-CTLA-4 antibody comprises a heavy chain variable region CDR2 (“CDRH2”) comprising an amino acid sequence set forth in SEQ ID NO: 12, wherein X can be an oxidized tryptophan.

In one embodiment, the anti-CTLA-4 antibody comprises a heavy chain variable region CDR3 (“CDRH3”) comprising an amino acid sequence with one or two amino acid variation(s) (“CDR variant”) to the amino acid sequence set forth in SEQ ID NO: 6.

In one embodiment, the anti-CTLA-4 antibody comprises a light chain variable region CDR1 (“CDRL1”) comprising an amino acid sequence with three or less, such as one or two amino acid variation(s) (“CDR variant”) to the amino acid sequence set forth in SEQ ID NO: 1.

In one embodiment, the anti-CTLA-4 antibody comprises a light chain variable region CDR1 (“CDRL1”) comprising an amino acid sequence set forth in SEQ ID NO: 11

In one embodiment, the anti-CTLA-4 antibody comprises a light chain variable region CDR2 (“CDRL2”) comprising an amino acid sequence with one or two amino acid variation(s) (“CDR variant”) to the amino acid sequence set forth in SEQ ID NO: 2.

In one embodiment, the anti-CTLA-4 antibody comprises a light chain variable region CDR3 (“CDRL3”) comprising an amino acid sequence with three or less, such as one or two amino acid variation(s) (“CDR variant”) to the amino acid sequence set forth in SEQ ID NO: 3.

In one embodiment, the anti-CTLA-4 antibody comprises a CDRH1 comprising an amino acid sequence with up to one amino acid variation to the amino acid sequence set forth in SEQ ID NO: 3; a CDRH2 comprising an amino acid sequence with up to five amino acid variations to the amino acid sequence set forth in SEQ ID NO: 4; a CDRH3 comprising an amino acid sequence with up to one amino acid variation to the amino acid sequence set forth in SEQ ID NO: 5; a CDRL1 comprising an amino acid sequence with up to three amino acid variations to the amino acid sequence set forth in SEQ ID NO: 1; a CDRL2 comprising an amino acid sequence with up to one amino acid variation to the amino acid sequence set forth in SEQ ID NO: 2; and/or a CDRL3 comprising an amino acid sequence with up to three amino acid variations to the amino acid sequence set forth in SEQ ID NO: 3.

In one embodiment, the anti-CTLA-4 antibody comprises a heavy chain variable region (“VH”) comprising an amino acid sequence with at least about 90%, 91%, 92%, 93%, 94%, 95%, 96%, 97%, 98%, 99% or 100% sequence identity to the amino acid sequence set forth in SEQ ID NO: 8. In one embodiment, the VH comprises an amino acid sequence with at least one amino acid variation to the amino acid sequence set forth in SEQ ID NO: 7, such as between 1 and 5, such as between 1 and 3, in particular up to 2 amino acid variations to the amino acid sequence set forth in SEQ ID NO: 8.

In one embodiment, the anti-CTLA-4 antibody comprises a light chain variable region (“VL”) comprising an amino acid sequence with at least about 90%, 91%, 92%, 93%, 94%, 95%, 96%, 97%, 98%, 99% or 100% sequence identity to the amino acid sequence set forth in SEQ ID NO: 7. In one embodiment, the VL comprises an amino acid sequence with at least one amino acid variation to the amino acid sequence set forth in SEQ ID NO: 8, such as between 1 and 5, such as between 1 and 3, in particular up to 2 amino acid variations to the amino acid sequence set forth in SEQ ID NO: 7.

In one embodiment, an anti-CTLA-4 antibody comprises a VH with the amino acid sequence set forth in SEQ ID NO: 8; and a VL with the amino acid sequence set forth in SEQ ID NO: 7.

In one embodiment, the anti-CTLA-4 antibody comprises a VH comprising an amino acid sequence with at least about 90%, 91%, 92%, 93%, 94%, 95%, 96%, 97%, 98%, 99% or 100% sequence identity to the amino acid sequence set forth in SEQ ID NO: 8; and a VL comprising an amino acid sequence with at least about 90%, 91%, 92%, 93%, 94%, 95%, 96%, 97%, 98%, 990% or 100% sequence identity to the amino acid sequence set forth in SEQ ID NO: 7.

In one embodiment, the anti-CTLA-4 antibody comprises a heavy chain sequence (“HC”) comprising an amino acid sequence with at least about 90%, 91%, 92%, 93%, 94%, 95%, 96%, 97%, 98%, 99% or 100% sequence identity to the amino acid sequence set forth in SEQ ID NO: 10. In one embodiment, the HC comprises an amino acid sequence with at least one amino acid variation to the amino acid sequence set forth in SEQ ID NO: 10, such as between 1 and 10, such as between 1 and 7, in particular up to 6 amino acid variations to the amino acid sequence set forth in SEQ ID NO: 10. In a further embodiment, the HC comprises one, two, three, four, five, six or seven amino acid variations to the amino acid sequence set forth in SEQ ID NO: 10.

In one embodiment, the anti-CTLA-4 antibody comprises a light chain region (“LC”) comprising an amino acid sequence with at least about 90%, 91%, 92%, 93%, 94%, 95%, 96%, 97%, 98%, 99% or 100% sequence identity to the amino acid sequence set forth in SEQ ID NO: 9. In one embodiment, the LC comprises an amino acid sequence with at least one amino acid variation to the amino acid sequence set forth in SEQ ID NO: 9, such as between 1 and 10, such as between 1 and 5, in particular up to 3 amino acid variations to the amino acid sequence set forth in SEQ ID NO: 9. In a further embodiment, the LC comprises one, two or three amino acid variations to the amino acid sequence set forth in SEQ ID NO: 9.

In one embodiment, the anti-CTLA-4 antibody comprises a HC comprising an amino acid sequence with at least about 90%, 91%, 92%, 93%, 94%, 95%, 96%, 97%, 98%, 99% or 100% sequence identity to the amino acid sequence set forth in SEQ ID NO: 10; and a LC comprising an amino acid sequence with at least about 90%, 91%, 92%, 93%, 94%, 95%, 96%, 97%, 98%, 99% or 100% sequence identity to the amino acid sequence set forth in SEQ ID NO: 9. Therefore, the antibody is an antibody with a heavy chain at least about 90% identical to the heavy chain amino acid sequence of SEQ ID NO: 10 and/or with a light chain at least about 90% identical to the light chain amino acid sequence of SEQ ID NO: 9.

In one embodiment, the antibody comprises a heavy chain sequence of SEQ ID NO: 10 and a light chain sequence of SEQ ID NO: 9. In one embodiment, the antibody is tremelimumab comprising a heavy chain sequence of SEQ ID NO: 10 and a light chain sequence of SEQ ID NO: 9.

### 3. Post-Translational Modification Products

The skilled person will appreciate that, upon production of an antibody, post-translational modifications may occur which produces a post-translational modification product. A “post-translational modification variant” of an antibody described herein is an antibody composition wherein all or a portion of the composition comprises a “post-translational modification”. Post-translational modifications are chemical changes to the antibody that may be the result from production of the antibody in a host cell, upstream and/or downstream manufacturing processes, and/or length of storage and storage conditions (e.g., effect of exposure to light, temperature, pH, water, or by reaction with an excipient and/or the immediate container closure system). Therefore, the composition of the disclosure may be formed from the manufacture or storage of antibodies of the disclosure. Exemplary post-translational modifications comprise antibody sequence changes (“antibody variant” as described above), cleavage of certain leader sequences, the addition of various sugar moieties in various glycosylation patterns including non-enzymatic glycosylation or glycation; deamidation; oxidation; disulfide bond scrambling and other cysteine variants, such as free sulfhydryls, racemized disulfides, thioethers and trisulfide bonds; isomerization; C-terminal lysine cleavage or clipping; and/or N-terminal glutamine cyclisation.

In one example, a post-translational modification product comprises a “product related impurity” that comprises a chemical change that results in reduced function and/or activity. In another example, a post-translational modification product comprises a “product related substance” that comprises a chemical change that does not result in reduced function and/or activity. Product related impurities for the anti-CTLA-4 antibodies described herein include oxidized variants and aggregated variants. Product related substances for the anti-CTLA-4 antibodies described herein include deamidated variants, isomerized variants, C-terminal cleaved variants and N-terminal pyro-glutamate variants.

In one embodiment, the anti-CTLA-4 antibody is tremelimumab comprising a heavy chain with the amino acid sequence set forth in SEQ ID NO: 10, and a light chain with the amino acid sequence set forth in SEQ ID NO: 9, comprising all functional post-translational modifications thereof.

The percent variant provided herein is expressed as a percentage of the total amount of antibody in the composition (e.g., a “population” of antibodies). For example, 50% or less oxidized variants is in the context of total antibody in the composition being 100%, of which 50% or less is oxidized; it does not include any other non-antibody substances present in the composition which may or may not be oxidized.

Antibody variants are commonly observed when the composition of antibodies is analyzed by charged based-separation techniques such as isoelectric focusing (IEF) gel electrophoresis, capillary isoelectric focusing (cIEF) gel electrophoresis, cation exchange chromatography (CEX) and anion exchange chromatography (AEX).

Post translational modifications can result in an increase or decrease in the net charge of the antibody and cause a decrease or increase in the pI value, thereby leading to acidic variants and basic variants (collectively called “charged variants”) with respect to the main isoform. The “main isoform” is the antibody population that elutes as the major peak on chromatograms and electropherograms. Acidic species are variants with lower apparent pI and basic species are variants with higher apparent pI, when antibodies are analyzed using IEF based methods. When analyzed by chromatography-based methods, acidic species and basic species are defined based on their retention times relative to the main peak. Acidic species are the variants that elute earlier than the main peak from CEX or later then than the main peak from AEX, while basic species are the variants that elute later than the main peak from CEX or earlier than the main peak from AEX. These methods separate the main isoform of the antibody from the acidic isoform (acidic variant) and basic isoform (basic variant).

The charged variant can be detected by various methods, such as ion exchange chromatography, for example, IEF (isoelectric focusing). The percent charged variant can be determined using capillary isoelectric focusing (cIEF). Capillary isoelectric focusing (cIEF) was used to measure the pI of tremelimumab and separate charge variants. The method can be used to quantitate the acidic and basic species as a percentage of the total area peak. The terms “species,” “isoform,” “form” and “peak” are used interchangeably to refer to the main isoform and the charged variant (acidic variant and basic variant).

In one aspect, the composition comprises an acidic variant of an anti-CTLA-4 antibody, wherein the acidic variant comprises a heavy chain amino acid sequence comprising a CDRH1 of SEQ ID NO: 4, a CDRH2 of SEQ ID NO: 5, and a CDRH3 of SEQ ID NO: 6, and a light chain amino acid sequence comprising a CDRL1 of SEQ ID NO: 1, a CDRL2 of SEQ ID NO: 2, and a CDRL3 of SEQ ID NO: 3; wherein the composition comprises <100% acidic variant.

In one embodiment, the composition comprises an acidic variant of an anti-CTLA-4 antibody, wherein the acidic variant comprises a heavy chain variable region at least about 90% identical to the amino acid sequence of SEQ ID NO: 8 and/or a light chain variable region at least about 90% identical to the amino acid sequence of SEQ ID NO: 7; wherein the composition comprises <100% acidic variant.

In another embodiment, the composition comprises an acidic variant of an anti-CTLA-4 antibody, wherein the acidic variant comprises a heavy chain at least about 90% identical to the amino acid sequence of SEQ ID NO: 10 and/or a light chain variable region at least about 90% identical to the amino acid sequence of SEQ ID NO: 9; wherein the composition comprises <100% acidic variant. In a yet further embodiment, the composition comprises an acidic variant of an anti-CTLA-4 antibody, wherein the acidic variant comprises a heavy chain sequence of SEQ ID NO: 10 and a light chain sequence of SEQ ID NO: 9; wherein the composition comprises <100% acidic variant. In one embodiment, the composition comprises an acidic variant of tremelimumab, wherein the acidic variant comprises a heavy chain sequence of SEQ ID NO: 10 and a light chain sequence of SEQ ID NO: 9; wherein the composition comprises <100% acidic variant.

In one aspect, the composition comprises <100% acidic variant. In one embodiment, the composition comprises <95%, <90%, <80%, <70%, <60%, <50%, <40%, <35%, <30% or <25% acidic variant. Alternatively, the composition comprises 5-100%, 5-90%, 5-80%, 5-70%, 5-60%, 5-50%, 5-40%, 5-35%, 5-30% or 5-25% acidic variant. Alternatively, the composition comprises 10-100%, 10-97%, 10-90%, 10-80%, 10-70%, 10-60%, 10-50%, 10-40%, 10-35%, 10-30% or 10-25% acidic variant. Alternatively, the composition comprises 20-100%, 20-97%, 20-90%, 20-800%, 20-70%, 20-60%, 20-50%, 20-40%, 20-35%, 20-30% or 20-25% acidic variant.

Alternatively, the composition comprises about 60%, about 50%, about 45%, about 40%, about 35%, about 30%, about 25%, about 20% or about 10% acidic variant.

In one aspect, the composition comprises a basic variant of an anti-CTLA-4 antibody, wherein the basic variant comprises a heavy chain amino acid sequence comprising a CDRH1 of SEQ ID NO: 4, a CDRH2 of SEQ ID NO: 5, and a CDRH3 of SEQ ID NO: 6, and a light chain amino acid sequence comprising a CDRL1 of SEQ ID NO: 1, a CDRL2 of SEQ ID NO: 2, and a CDRL3 of SEQ ID NO: 3; wherein the composition comprises <100% basic variant.

In one embodiment, the composition comprises a basic variant of an anti-CTLA-4 antibody, wherein the basic variant comprises a heavy chain variable region at least about 90% identical to the amino acid sequence of SEQ ID NO: 8 and/or a light chain variable region at least about 90% identical to the amino acid sequence of SEQ ID NO: 7; wherein the composition comprises <100% of basic variant.

In one aspect, the composition comprises <100% basic variant. In one embodiment, the composition comprises <95%, <90%, <80%, <70%, <60%, <50%, <40%, <35%, <30% or <25% basic variant. Alternatively, the composition comprises 5-100%, 5-90%, 5-800%, 5-70%, 5-60%, 5-50%, 5-40%, 5-35%, 5-30% or 5-25% basic variant. Alternatively, the composition comprises 10-100%, 10-97%, 10-90%, 10-80%, 10-70%, 10-60%, 10-50%, 10-40%, 10-35%, 10-30% or 10-25% basic variant. Alternatively, the composition comprises 20-100%, 20-97%, 20-90%, 20-80%, 20-70%, 20-60%, 20-50%, 20-40%, 20-35%, 20-30% or 20-25% a basic variant.

Alternatively, the composition comprises about 60%, about 50%, about 45%, about 40%, about 35%, about 30%, about 25%, about 20% or about 10% basic variant.

In one embodiment, the composition has at least 70% bioassay potency compared to a reference standard bioassay potency.

In another embodiment, the composition comprises a basic variant of an anti-CTLA-4 antibody, wherein the basic variant comprises a heavy chain at least about 90% identical to the amino acid sequence of SEQ ID NO: 10 and/or a light chain at least about 90% identical to the amino acid sequence of SEQ ID NO: 9; wherein the composition comprises <100% of basic variant. In a yet further embodiment, the composition comprises a basic variant of an anti-CTLA-4 antibody, wherein the basic variant comprises a heavy chain sequence of SEQ ID NO: 10 and a light chain sequence of SEQ ID NO: 9; wherein the composition comprises <100% basic variant. In one embodiment, the composition comprises a basic variant of tremelimumab, wherein the basic variant comprises a heavy chain sequence of SEQ ID NO: 10 and a light chain sequence of SEQ ID NO: 9; wherein the composition comprises <100% basic variant.

In one aspect, the composition comprises a main isoform of an anti-CTLA-4 antibody, wherein the main isoform comprises a heavy chain amino acid sequence comprising a CDRH1 of SEQ ID NO: 3, a CDRH2 of SEQ ID NO: 4, and a CDRH3 of SEQ ID NO: 5, and a light chain amino acid sequence comprising a CDRL1 of SEQ ID NO: 1, a CDRL2 of SEQ ID NO: 2, and a CDRL3 of SEQ ID NO: 3; wherein the composition comprises >1% main isoform.

In one embodiment, the composition comprises a main isoform of an anti-CTLA-4 antibody, wherein the main isoform comprises a heavy chain variable region at least about 90% identical to the amino acid sequence of SEQ ID NO: 7 and/or a light chain variable region at least about 90% identical to the amino acid sequence of SEQ ID NO: 8; wherein the composition comprises >1% main isoform.

In another embodiment, the composition comprises a main isoform of an anti-CTLA-4 antibody, wherein the main isoform comprises a heavy chain at least about 90% identical to the amino acid sequence of SEQ ID NO: 10 and/or a light chain variable region at least about 90% identical to the amino acid sequence of SEQ ID NO: 9; wherein the composition comprises >1% main isoform. In a yet further embodiment, the composition comprises a main isoform of an anti-CTLA-4 antibody, wherein the main isoform comprises a heavy chain sequence of SEQ ID NO: 10 and a light chain sequence of SEQ ID NO: 9; wherein the composition comprises >1% main isoform.

In one embodiment, the composition comprises a main isoform of tremelimumab, wherein the main isoform comprises a heavy chain sequence of SEQ ID NO: 10 and a light chain sequence of SEQ ID NO: 9; wherein the composition comprises >1% main isoform.

In one aspect, the composition comprises >1% main isoform. In one embodiment, the composition comprises >2.6%, >3%, >5%, >10%, >20%, >30%, >40%, >50%, >55%, >60%, >65%, >70%, >75%, >80% or >90% main isoform. In one embodiment, the composition comprises 2-90%, 2-80%, 2-75%, 5-90%, 10-90%, 20-90%, 30-90° %, 40-90%, 50-90% or 60-90% main isoform. In one embodiment, the composition comprises 5-80%, 10-80%, 20-80%, 30-80%, 40-80%, 50-80% or 60-80% main isoform. Alternatively, the composition comprises about 80%, about 75%, about 70%, about 65%, about 600%, about 500% or about 55% main isoform.

The percent acidic variant, percent basic variant and percent main isoform can be determined using capillary isoelectric focusing (cIEF). It is understood that these isoform/charged variant embodiments may be combined with any one or a combination of antibody variants described herein.

In one aspect, the composition comprises a charged variant of an anti-CTLA-4 antibody comprising a heavy chain amino acid sequence comprising a CDRH1 of SEQ ID NO: 3, a CDRH2 of SEQ ID NO: 4, and a CDRH3 of SEQ ID NO: 5, and a light chain amino acid sequence comprising a CDRL1 of SEQ ID NO: 1, a CDRL2 of SEQ ID NO: 2, and a CDRL3 of SEQ ID NO: 3; wherein the composition comprises: <100% acidic variant; and/or <100% basic variant; and/or >1% main isoform.

In another aspect, the composition comprises a charged variant of an anti-CTLA-4 antibody comprising a heavy chain amino acid sequence comprising a CDRH1 of SEQ ID NO: 3, a CDRH2 of SEQ ID NO: 4, and a CDRH3 of SEQ ID NO: 5, and a light chain amino acid sequence comprising a CDRL1 of SEQ ID NO: 1, a CDRL2 of SEQ ID NO: 2, and a CDRL3 of SEQ ID NO: 3; wherein the composition comprises: 4-97% acidic variant; and/or 10-97% basic variant; and/or 2-80% main isoform.

In another aspect, the composition comprises a charged variant of an anti-CTLA-4 antibody comprising a heavy chain amino acid sequence comprising a CDRH1 of SEQ ID NO: 3, a CDRH2 of SEQ ID NO: 4, and a CDRH3 of SEQ ID NO: 5, and a light chain amino acid sequence comprising a CDRL1 of SEQ ID NO: 1, a CDRL2 of SEQ ID NO: 2, and a CDRL3 of SEQ ID NO: 3; wherein the composition comprises: <35% acidic variant; and/or <35% basic variant; and/or >55% main isoform.

In another aspect, the composition comprises a charged variant of an anti-CTLA-4 antibody comprising a heavy chain amino acid sequence comprising a CDRH1 of SEQ ID NO: 3, a CDRH2 of SEQ ID NO: 4, and a CDRH3 of SEQ ID NO: 5, and a light chain amino acid sequence comprising a CDRL1 of SEQ ID NO: 1, a CDRL2 of SEQ ID NO: 2, and a CDRL3 of SEQ ID NO: 3; wherein the composition comprises: 4-30% acidic variant; and/or 10-30% basic variant; and/or 60-80% main isoform.

In one aspect, the composition comprises a charged variant of an anti-CTLA-4 antibody comprising a heavy chain amino acid sequence comprising a VH of SEQ ID NO: 8, and a light chain amino acid sequence comprising a VL of SEQ ID NO: 7; wherein the composition comprises: <100% acidic variant; and/or <100% basic variant; and/or >1% main isoform. In one embodiment, the composition comprises: 10-97% acidic variant; and/or 10-97% basic variant; and/or 2-80% main isoform. In an alternative embodiment, the composition comprises: 10-30% acidic variant; and/or 10-30% basic variant; and/or 60-80% main isoform. In a further embodiment, the composition comprises: <35% acidic variant; and/or <35% basic variant; and/or >55% main isoform. In one aspect, the composition comprises a charged variant of an anti-CTLA-4 antibody comprising a heavy chain amino acid sequence of SEQ ID NO: 10, and a light chain amino acid sequence of SEQ ID NO: 9; wherein the composition comprises: <100% acidic variant; and/or <100% basic variant; and/or >1% main isoform. In one embodiment, the composition comprises: 10-97% acidic variant; and/or 10-97% basic variant; and/or 2-80% main isoform. In an alternative embodiment, the composition comprises: 10-30% acidic variant; and/or 10-30% basic variant; and/or 60-80% main isoform. In a further embodiment, the composition comprises: <35% acidic variant; and/or <35% basic variant; and/or >55% main isoform.

In one embodiment, the composition has at least 70% bioassay potency compared to a reference standard bioassay potency.

Oxidation can occur during production and/or storage (i.e., in the presence of oxidizing conditions) and results in a covalent modification of a protein, induced either directly by reactive oxygen species or indirectly by reaction with secondary byproducts of oxidative stress. Oxidation may happen primarily with methionine residues, but may also occur at tryptophan and free cysteine residues. Oxidation can occur in a CDR, in a Fab (non-CDR) region, or in an Fc region.

In one aspect, the composition comprises an antibody comprising an oxidation post-translational modification (“oxidation” or “oxidized”), also referred to herein as an “oxidized variant”. The variant may comprise an oxidized amino acid residue in the heavy chain sequence and/or the light chain sequence, such as a CDR of the heavy chain sequence and/or a CDR of the light chain sequence. The oxidized variant may be present in one or both chains of the heavy chain or light chain.

In one aspect, the composition comprises an oxidized variant of an anti-CTLA-4 antibody, wherein the oxidized variant comprises a heavy chain amino acid sequence comprising a CDRH1 of SEQ ID NO: 3, a CDRH2 of SEQ ID NO: 4, and a CDRH3 of SEQ ID NO: 5, and a light chain amino acid sequence comprising a CDRL1 of SEQ ID NO: 1, a CDRL2 of SEQ ID NO: 2, and a CDRL3 of SEQ ID NO: 3; wherein the composition comprises <40% of oxidized variant.

In one aspect, the composition comprises a population of anti-CTLA-4 antibodies that includes: antibodies having a heavy chain amino acid sequence comprising SEQ ID NO: 3 (CDRH1), SEQ ID NO: 4 (CDRH2), and SEQ ID NO: 5 (CDRH3) and a light chain amino acid sequence comprising SEQ ID NO: 1 (CDRL1), SEQ ID NO: 2 (CDRL2) and SEQ ID NO: 3 (CDRL3), and oxidized variants thereof, wherein <40% of the population of antibodies is comprised of the oxidized variants.

In one embodiment, the oxidized variant comprises oxidation at a methionine and/or tryptophan residue in a CDR of the heavy chain sequence and/or a CDR of the light chain sequence. In one embodiment, the oxidized variant comprises oxidation at a methionine and/or tryptophan residue in any one of SEQ ID NOs: 1-6. In a further embodiment, the antibody comprises oxidation at a methionine residue in a CDR of the heavy chain sequence, such as CDRH1 and/or CDRH3. In a further embodiment, the antibody comprises oxidation at a tryptophan residue in a CDR of the light chain sequence, such as CDRL2. In some embodiments, the oxidized variant comprises one or a combination of oxidation at: W52 of CDRH2, M256, M362, M401 and/or M432 of the Full Heavy chain.

It will be understood that reference to a position in the CDR (e.g., W52) provides the position number in relation to the entire antibody sequence (sequential numbering). Therefore, it will be understood that W52 of CDRH2 refers to the third residue of SEQ ID NO: 5.

In one embodiment, the antibody comprises oxidation at a methionine and/or tryptophan residue in the Fc region of the heavy chain sequence and/or the Fc region of the light chain sequence. In some embodiments, the oxidized variant comprises one or a combination of oxidation at: M256, M362, M401 and/or M432 of the Fc region of the heavy chain sequence.

In one aspect, the composition comprises an antibody that is at least about 900% identical to the heavy chain amino acid sequence of SEQ ID NO: 10 and/or at least about 90% identical to the light chain sequence of SEQ ID NO: 9, and comprises oxidation in the heavy chain sequence, for example, oxidation at amino acid W52 of CDRH2, M256 of the Fc, M362 of the Fc region, M401 of the Fc region and/or M432 of the Fc region. In one embodiment, the composition comprises an antibody that is at least about 90% identical to the heavy chain amino acid sequence of SEQ ID NO: 10 and/or at least about 90% identical to the light chain sequence of SEQ ID NO: 9.

In one embodiment, the antibody comprises a heavy chain variable region at least about 90% identical to the amino acid sequence of SEQ ID NO: 8 and/or a light chain variable region at least about 90% identical to the amino acid sequence of SEQ ID NO: 7. In a further embodiment, the antibody is at least about 90% identical to the heavy chain amino acid sequence of SEQ ID NO: 10 and/or at least about 90% identical to the light chain amino acid sequence of SEQ ID NO: 9. In a yet further embodiment, the antibody comprises a heavy chain sequence of SEQ ID NO: 10 and a light chain sequence of SEQ ID NO: 9.

In one aspect, the composition comprises an anti-CTLA-4 antibody having a heavy chain sequence comprising a CDRH1 comprising the amino acid sequence of SEQ ID NO: 4, a CDRH2 comprising the amino acid sequence of SEQ ID NO: 5, and a CDRH3 comprising the amino acid sequence of SEQ ID NO: 6, and a light chain sequence comprising a CDRL1 comprising the amino acid sequence of SEQ ID NO: 1, a CDRL2 comprising the amino acid sequence of SEQ ID NO: 2, and a CDRL3 comprising the amino acid sequence of SEQ ID NO: 3; wherein the composition comprises <35% oxidized variant.

In one aspect, the composition comprises an anti-CTLA-4 antibody comprising a heavy chain variable region at least about 90% identical to the amino acid sequence of SEQ ID NO: 8 and/or a light chain variable region at least about 90% identical to the amino acid sequence of SEQ ID NO: 7, wherein the composition comprises <35% oxidized variant. In a further aspect, the composition comprises an anti-CTLA-4 antibody comprising a heavy chain variable region of SEQ ID NO: 8 and/or a light chain variable region of SEQ ID NO: 7, wherein the composition comprises <35% oxidized variant.

In one aspect, the composition comprises a population of anti-CTLA-4 antibodies that includes: antibodies having a heavy chain variable region as set forth in SEQ ID NO: 8 and a light chain variable region as set forth in SEQ ID NO: 7, and oxidized variants thereof, wherein <35% of the population of antibodies is comprised of the oxidized variants.

In one aspect, the composition comprises an anti-CTLA-4 antibody comprising a heavy chain sequence at least about 90% identical to the amino acid sequence of SEQ ID NO: 10 and/or a light chain sequence at least about 90% identical to the amino acid sequence of SEQ ID NO: 9, wherein the composition comprises <35% oxidized variant.

In one aspect, the composition comprises an anti-CTLA-4 antibody comprising a heavy chain sequence of SEQ ID NO: 10 and a light chain sequence of SEQ ID NO: 9, wherein the composition comprises <35% oxidized variant.

In one embodiment, the composition comprises an oxidized variant of tremelimumab, wherein the oxidized variant comprises a heavy chain sequence of SEQ ID NO: 10 and a light chain sequence of SEQ ID NO: 9; wherein the composition comprises <45% oxidized variant.

In one embodiment, the composition comprises an oxidized variant of tremelimumab, wherein the oxidized variant comprises a heavy chain sequence of SEQ ID NO: 10 and a light chain sequence of SEQ ID NO: 9; wherein the composition comprises an amount of oxidized variant in the range of 0.1% to 35%.

In one aspect, the composition comprises (a) an antibody having a heavy chain sequence of SEQ ID NO: 10 and a light chain sequence of SEQ ID NO: 9, and (b) an antibody having a heavy chain sequence at least 90% identical to SEQ ID NO: 10 and a light chain sequence at least 90% identical to SEQ ID NO: 9, wherein the composition comprises <35% oxidized variant.

In one aspect, the composition comprises a population of anti-CTLA-4 antibodies that includes: antibodies having a heavy chain amino acid sequence as set forth in SEQ ID NO: 10 and a light chain amino acid sequence as set forth in SEQ ID NO: 9, and oxidized variants thereof, wherein <35% of the population of antibodies is comprised of the oxidized variants.

In one aspect, the composition comprises (a) an antibody having a heavy chain sequence of SEQ ID NO: 10 and a light chain sequence of SEQ ID NO: 9, and (b) an antibody having a heavy chain sequence at least 90% identical to SEQ ID NO: 10 and a light chain sequence at least 90% identical to SEQ ID NO: 9; and (c) an oxidized variant of the antibody of (a) and/or (b), wherein the oxidized variant is selected from any one or a combination of <30% oxidation at W52 of the heavy chain, <35% oxidation at M256 of the heavy chain, <12.5% oxidation at M362 of the heavy chain, <16% oxidation at M401 of the heavy chain, and/or <350% oxidation at M432 of the heavy chain.

In one aspect, the composition comprises <35% oxidized variant. In one embodiment, the composition comprises <35%, <30%, <20%, <15%, <10%, <5%, <4%, or <3% oxidized variant. In one embodiment, the composition comprises 0.01-35%, 0.01-30%, 0.01-20%, 0.01-15%, 0.01-10%, 0.01-5%, 0.01-4%, or 0.01-3% oxidized variant.

Alternatively, the composition comprises 0.05-35%, 0.05-30%, 0.05-20%, 0.05-15%, 0.05-10%, 0.05-5%, 0.05-4%, or 0.05-3% oxidized variant. Alternatively, the composition comprises 0.5-35%, 0.5-30%, 0.5-20%, 0.5-15%, 0.5-10%, 0.5-5%, 0.5-4%, or 0.5-3% oxidized variant. Alternatively, the composition comprises 1-35%, 1-300%, 1-20%, 1-15%, 1-10%, 1-5%, 1-4%, 1-3%, 2-4%, or 2-3% oxidized variant. Alternatively, the composition comprises about 10%, about 5%, about 4%, about 3%, about 2%, or about 1% oxidized variant. It will be understood that these oxidized variant embodiments may be combined with any one of the antibody variants described herein.

In one embodiment, the composition comprises one or a combination of: <35% oxidation at M256 of the heavy chain, <12.5% oxidation at M362 of the heavy chain, <16% oxidation of M401 of the heavy chain, <35% oxidation at M432 of the heavy chain, and/or <300% oxidation at W52 of CDRH2.

In one aspect, the composition comprises: (a) an antibody having a heavy chain sequence of SEQ ID NO: 10 and a light chain sequence of SEQ ID NO: 9, and (b) an oxidized variant of the antibody selected from any one or a combination of <25% oxidation at W52 of the heavy chain, <30% oxidation at M256 of the heavy chain, <10% oxidation at M362 of the heavy chain, <12% oxidation of M401 of the heavy chain, and/or <30% oxidation at M432 of the heavy chain.

In one embodiment, the composition comprises <25% oxidation at W52 of the heavy chain sequence. In one embodiment, the composition comprises <25%, <20%, <15%, <10%, <7.5%, <5%, <4%, <3%, <2%, or <1% oxidation at W52 of the heavy chain sequence. Alternatively, the composition comprises 0-25%, 0-20%, 0-15%, 0-10%, 0-7.5%, 0-5%, 0-4%, 0-3%, 0-2% or 0-1% oxidation at W52 of the heavy chain sequence. In one embodiment, the composition comprises 0.01-25%, 0.01-20%, 0.01-15%, 0.01-10%, 0.01-7.5%, 0.01-5%, 0.01-4%, 0.01-3%, 0.01-2%, or 0.01-1% oxidation at W52 of the heavy chain sequence. In one embodiment, the composition comprises 0.05-25%, 0.05-20%, 0.05-150%, 0.05-100%, 0.05-7.5%, 0.05-5%, 0.05-4%, 0.05-3%, 0.05-2%, or 0.05-1% oxidation at W52 of the heavy chain sequence. Alternatively, the composition comprises 0.5-25%, 0.5-20%, 0.5-15%, 0.5-10%, 0.5-7.5%, 0.5-5%, 0.5-4%, or 0.5-3%, 0.5-2% or 0.5-1% oxidation at W52 of the heavy chain sequence. Alternatively, the composition comprises 0.1% or more and 25% or less oxidation at W52 of the heavy chain sequence. Alternatively, the composition comprises about 10%, about 5%, about 4%, about 3%, about 2%, or about 1% oxidation at W52 of the heavy chain sequence. As shown by the data presented herein, thermal stress forced degradation producing up to 4.5% oxidation at W52 gives 98% potency.

In one aspect, the composition comprises an antibody having a heavy chain sequence of SEQ ID NO: 10 and a light chain sequence of SEQ ID NO: 9, wherein the composition comprises <25% oxidation at W52 of the heavy chain sequence. In one embodiment, the composition comprises an oxidized variant of tremelimumab, wherein the oxidized variant comprises a heavy chain sequence of SEQ ID NO: 10 and a light chain sequence of SEQ ID NO: 9; wherein the composition comprises <25% oxidation at W52 of the heavy chain sequence.

In one embodiment, the composition comprises <35% oxidation at M256 of the heavy chain sequence. In one embodiment, the composition comprises <35%, <30%, <25%, <20%, <16%, <15%, <12.5%, <10%, <7.5%, <5%, <4%, <3%, <2%, or <1% oxidation at M256 of the heavy chain sequence. Alternatively, the composition comprises 0-35%, 0-30%, 0-25%, 0-20%, 0-16%, 0-15%, 0-12.5%, 0-10%, 0-7.5%, 0-5%, 0-4%, 0-3%, 0-2% or 0-1% oxidation at M256 of the heavy chain sequence. In one embodiment, the composition comprises 0.01-35%, 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, or 0.01-1% oxidation at M256 of the heavy chain sequence. Alternatively, the composition comprises 0.5-35%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2% or 0.5-1% oxidation at M256 of the heavy chain sequence. Alternatively, the composition comprises 0.1% or more and 35% or less oxidation at M256 of the heavy chain sequence. Alternatively, the composition comprises about 30%, about 25%, about 20%, 10%, about 5%, about 4%, about 3%, about 2%, or about 1% oxidation at M256 of the heavy chain sequence. As shown by the data presented herein, H₂O₂ forced degradation producing up to 68.5% oxidation at M34 gives 83 potency in bioassay. Extrapolating from the H₂O₂ forced degradation data for oxidation at HC Met256, up to 13.4% oxidation can result in at least 88% potency. In one aspect, the composition comprises an antibody having a heavy chain sequence of SEQ ID NO: 10 and a light chain sequence of SEQ ID NO: 9, wherein the composition comprises <13.4% oxidation at M256 of the heavy chain sequence. In one embodiment, the composition comprises an oxidized variant of tremelimumab, wherein the oxidized variant comprises a heavy chain sequence of SEQ ID NO: 10 and a light chain sequence of SEQ ID NO: 9; wherein the composition comprises <13.4% oxidation at M256 of the heavy chain sequence.

In one embodiment, the composition comprises <16% oxidation at M401 of the heavy chain sequence. In one embodiment, the composition comprises <16%, <15%, <10%, <5%, <2%, or <1% oxidation at M401 of the heavy chain sequence. In one embodiment, the composition comprises 0-16%, 0-15%, 0-10%, 0-5%, 0-4%, 0-3%, 0-2%, or 0-1% oxidation at M401 of the heavy chain sequence. In one embodiment, the composition comprises 0.01-16%, 0.01-15%, 0.01-10%, 0.01-5%, 0.01-4%, 0.01-3)%, 0.01-2%, or 0.01-1% oxidation at M401 of the heavy chain sequence. Alternatively, the composition comprises 0.5-16%, 0.5-15%, 0.5-10%, 0.5-5%, 0.5-4%, 0.5-3%, 0.5-2% or 0.5-1% oxidation at M401 of the heavy chain sequence. Alternatively, the composition comprises 0.1% or more, or 16% or less oxidation at M401 of the heavy chain sequence. Alternatively, the composition comprises about 10%, about 5%, about 4%, about 3%, about 2%, or about 1% oxidation at M401 of the heavy chain sequence. As shown by the data presented herein, H₂O₂ forced degradation producing up to 55.2% oxidation at M401 gives 83% potency in bioassay.

In one aspect, the composition comprises an antibody having a heavy chain sequence of SEQ ID NO: 10 and a light chain sequence of SEQ ID NO: 9, wherein the composition comprises <16% oxidation at M401 of the heavy chain sequence. In one embodiment, the composition comprises an oxidized variant of tremelimumab, wherein the oxidized variant comprises a heavy chain sequence of SEQ ID NO: 10 and a light chain sequence of SEQ ID NO: 9; wherein the composition comprises <16% oxidation at M401 of the heavy chain sequence.

In one embodiment, the composition comprises <12.5% oxidation at M362 of the heavy chain sequence. In one embodiment, the composition comprises <12.5%, <10%, <5%, <4%, or <3% oxidation at M362 of the heavy chain sequence. In one embodiment, the composition comprises 0.01-12.5%, 0.01-10%, 0.01-5%, 0.01-4%, 0.01-3%, 0.01-2% or 0.01-1% oxidation at M3628 of the heavy chain sequence. Alternatively, the composition comprises 0.5-12.5%, 0.5-10%, 0.5-5%, 0.5-4%, or 0.5-3% oxidation at M362 of the heavy chain sequence. Alternatively, the composition comprises 1-12.5%, 1-10%, 1-5%, 1-4%, 1-3%, 2-4%, or 2-3% oxidation at M362 of the heavy chain sequence. Alternatively, the composition comprises 1% or more and 12.5% or less oxidation at M362 of the heavy chain sequence. Alternatively, the composition comprises about 10%, about 5%, about 4%, about 3%, about 2%, or about 1% oxidation at M362 of the heavy chain sequence. As shown by the data presented herein, H₂O₂ forced degradation producing up to 26.5% oxidation at M362 gives 83% potency in bioassay. It is therefore expected that oxidation at M362 can go higher than 26.5% without any impact to relative potency.

In one embodiment, the composition comprises <35% oxidation at M432 of the heavy chain sequence. In one embodiment, the composition comprises <35%, <30%, <20%, <15%, <10%, <5%, <2%, or <1% oxidation at M432 of the heavy chain sequence. In one embodiment, the composition comprises 0.01-35%, 0.01-30%, 0.01-20%, 0.01-15%, 0.01-10%, 0.01-5%, 0.01-4%, 0.01-3%, 0.01-2% or 0.01-1% oxidation at M432 of the heavy chain sequence. Alternatively, the composition comprises 0.5-35%, 0.5-30%, 0.5-20%, 0.5-15%, 0.5-10%, 0.5-5%, 0.5-4%, or 0.5-3% oxidation at M432 of the heavy chain sequence. Alternatively, the composition comprises 0.1% or more and 35% or less oxidation at M432 of the heavy chain sequence. Alternatively, the composition comprises about 10%, about 5%, about 4%, about 3%, about 2%, or about 1% oxidation at M432 of the heavy chain sequence. As shown by the data presented herein, H₂O₂ forced degradation producing up to 42.1% oxidation at M432 gives 83% potency in bioassay (i.e., within assay variability—full function) and photolysis forced degradation up to 20.7% oxidation at M432 gives 88% potency in bioassay. It is therefore expected that oxidation at M432 can go higher than 42.1% without any impact to relative potency.

In one embodiment, the composition comprises an oxidized variant of tremelimumab, wherein the oxidized variant comprises a heavy chain sequence of SEQ ID NO: 10 and a light chain sequence of SEQ ID NO: 9; wherein the composition comprises <35% oxidation at M256 and/or M362 and/or M401 and/or M432 of the heavy chain sequence.

In one aspect, the composition comprises an anti-CTLA-4 antibody comprising a heavy chain amino acid sequence comprising a CDRH1 of SEQ ID NO: 4, a CDRH2 of SEQ ID NO: 5, and a CDRH3 of SEQ ID NO: 6, and a light chain amino acid sequence comprising a CDRL1 of SEQ ID NO: 1, a CDRL2 of SEQ ID NO: 2, and a CDRL3 of SEQ ID NO: 3; wherein the composition comprises: <100% acidic variant; and/or <100% basic variant; and/or >1% main isoform; and/or <35% oxidized variant.

In another aspect, the composition comprises an anti-CTLA-4 antibody comprising a heavy chain amino acid sequence comprising a CDRH1 of SEQ ID NO: 4, a CDRH2 of SEQ ID NO: 5, and a CDRH3 of SEQ ID NO: 6, and a light chain amino acid sequence comprising a CDRL1 of SEQ ID NO: 1, a CDRL2 of SEQ ID NO: 2, and a CDRL3 of SEQ ID NO: 3; wherein the composition comprises: 5-45% acidic variant; and/or 5-97% basic variant; and/or 2-80% main isoform; and/or <35% oxidized variant.

In one example, oxidation can be determined using tryptic peptide mapping tandem mass spectrometry (peptide mapping LC-MS/MS).

In one aspect, the composition comprises antibodies that are aggregated antibodies (High molecular weight (HMW) species) also referred to herein as an “aggregated variant”. The aggregated antibodies may comprise dimers or higher order structures formed of antibody monomers and subunits thereof. High molecular weight (HMW) species may therefore be comprised of dimerized antibodies and monomers with additional subunits (such as a monomer with two light chain subunits, or an LC-LC dimer that is non-covalently bound to the monomer). Aggregated variants can be, for example, covalent or non-covalent, reducible or non-reducible, and visible or subvisible aggregates of an antibody disclosed herein. Aggregated variants can be characterized and distinguished from an antibody based on their size. For example, the size distribution of an antibody composition can be detected using size exclusion chromatography (SEC). In one aspect, the composition comprises an anti-CTLA-4 antibody having a heavy chain sequence comprising a CDRH1 comprising the amino acid sequence of SEQ ID NO: 4, a CDRH2 comprising the amino acid sequence of SEQ ID NO: 5, and a CDRH3 comprising the amino acid sequence of SEQ ID NO: 6, and a light chain sequence comprising a CDRL1 comprising the amino acid sequence of SEQ ID NO: 1, a CDRL2 comprising the amino acid sequence of SEQ ID NO: 2, and a CDRL3 comprising the amino acid sequence of SEQ ID NO: 3; wherein the composition comprises <26% aggregated variant. It will be understood that these aggregated variant embodiments may be combined with any of the antibody variants described herein.

In one aspect, the composition comprises an aggregated variant of an anti-CTLA-4 antibody, wherein the aggregated variant comprises a heavy chain sequence comprising a CDRH1 of SEQ ID NO: 4, a CDRH2 of SEQ ID NO: 5, and a CDRH3 of SEQ ID NO: 6, and a light chain sequence comprising a CDRL1 of SEQ ID NO: 1, a CDRL2 of SEQ ID NO: 2, and a CDRL3 of SEQ ID NO: 3; wherein the composition comprises <26% aggregated variant.

In one aspect, the composition comprises a population of anti-CTLA-4 antibodies that includes: antibodies having a heavy chain amino acid sequence comprising SEQ ID NO: 4 (CDRH1), SEQ ID NO: 5 (CDRH2), and SEQ ID NO: 6 (CDRH3) and a light chain amino acid sequence comprising SEQ ID NO: 1 (CDRL1), SEQ ID NO: 2 (CDRL2) and SEQ ID NO: 3 (CDRL3), and aggregated variants thereof, wherein ≤26% of the population of antibodies is comprised of the aggregated variants.

In one embodiment, the antibody comprises a heavy chain variable region at least about 90% identical to the amino acid sequence of SEQ ID NO: 8 and/or a light chain variable region at least about 90% identical to the amino acid sequence of SEQ ID NO: 9. In a further embodiment, the antibody is at least about 90% identical to the heavy chain amino acid sequence of SEQ ID NO: 10 and/or at least about 90% identical to the light chain amino acid sequence of SEQ ID NO: 9. In a yet further embodiment, the antibody comprises a heavy chain sequence of SEQ ID NO: 10 and a light chain sequence of SEQ ID NO: 9.

In one aspect, the composition comprises an anti-CTLA-4 antibody comprising a heavy chain variable region at least about 90% identical to the amino acid sequence of SEQ ID NO: 8 and/or a light chain variable region at least about 90% identical to the amino acid sequence of SEQ ID NO: 7, wherein the composition comprises <26% aggregated variant. In a further aspect, the composition comprises an anti-CTLA-4 antibody comprising a heavy chain variable region of SEQ ID NO: 8 and/or a light chain variable region of SEQ ID NO: 7, wherein the composition comprises <26% aggregated variant.

In one aspect, the composition comprises a population of anti-CTLA-4 antibodies that includes: antibodies having a heavy chain variable region as set forth in SEQ ID NO: 8 and a light chain variable region as set forth in SEQ ID NO: 7, and aggregated variants thereof, wherein <26% of the population of antibodies is comprised of the aggregated variants.

In one aspect, the composition comprises an anti-CTLA-4 antibody comprising a heavy chain sequence at least about 90% identical to the amino acid sequence of SEQ ID NO: 10 and/or a light chain sequence at least about 90% identical to the amino acid sequence of SEQ ID NO: 9, wherein the composition comprises <26% aggregated variant.

In one aspect, the composition comprises an antibody having a heavy chain sequence of SEQ ID NO: 10 and a light chain sequence of SEQ ID NO: 9, and an antibody having a heavy chain sequence at least about 90% identical to the amino acid sequence of SEQ ID NO: 10 and/or a light chain sequence at least about 90% identical to the amino acid sequence of SEQ ID NO: 9, wherein the composition comprises <26% aggregated variant.

In one aspect, the composition comprises an anti-CTLA-4 antibody comprising a heavy chain sequence of SEQ ID NO: 10 and a light chain sequence of SEQ ID NO: 9, wherein the composition comprises <26% aggregated variant. In one embodiment, the composition comprises an aggregated variant of tremelimumab, wherein the aggregated variant comprises a heavy chain sequence of SEQ ID NO: 10 and a light chain sequence of SEQ ID NO: 9; wherein the composition comprises <26% aggregated variant. In one embodiment, the composition comprises an aggregated variant of tremelimumab, wherein the aggregated variant comprises a heavy chain sequence of SEQ ID NO: 10 and a light chain sequence of SEQ ID NO: 9; wherein the composition comprises aggregated variant in the range of 0.01% and 26% aggregated variant.

In one aspect, the composition comprises a population of anti-CTLA-4 antibodies that includes: antibodies having a heavy chain amino acid sequence as set forth in SEQ ID NO: 10 and a light chain amino acid sequence as set forth in SEQ ID NO: 9, and aggregated variants thereof, wherein <26% of the population of antibodies is comprised of the aggregated variants.

The antibody composition may comprise <26% aggregated variants, such as <26%, <25%, <20%, <10%, <5%, <4%, <3%, <2%, or <1% aggregated variants. In another embodiment, the composition may comprise 0.01-26%, 0.01-25%, 0.01-20%, 0.01-10%, 0.01-5%, 0.01-4%, 0.01-3%, 0.01-2%, or 0.01-1% aggregated variants. Alternatively, the composition comprises more than 1% and less than 26% aggregated variants. Alternatively, the composition may comprise about 10%, about 5%, about 4%, about 3%, about 2%, or about 1% aggregated variants. As shown by the data presented herein, combined acid treated and thermal forced degradation producing up to 25.9% of the composition comprising of aggregated antibodies gives 91% potency in bioassay (i.e., within assay variability—full function).

In one aspect, the composition comprises an anti-CTLA-4 antibody comprising a heavy chain amino acid sequence comprising a CDRH1 of SEQ ID NO: 4, a CDRH2 of SEQ ID NO: 5, and a CDRH3 of SEQ ID NO: 6, and a light chain amino acid sequence comprising a CDRL1 of SEQ ID NO: 1, a CDRL2 of SEQ ID NO: 2, and a CDRL3 of SEQ ID NO: 3; wherein the composition comprises: <100% acidic variant; and/or <100% basic variant; and/or >1% main isoform; and/or <35% oxidized variant; and/or <26% aggregated variant.

In another aspect, the composition comprises an anti-CTLA-4 antibody comprising a heavy chain amino acid sequence comprising a CDRH1 of SEQ ID NO: 4, a CDRH2 of SEQ ID NO: 5, and a CDRH3 of SEQ ID NO: 6, and a light chain amino acid sequence comprising a CDRL1 of SEQ ID NO: 1, a CDRL2 of SEQ ID NO: 2, and a CDRL3 of SEQ ID NO: 3; wherein the composition comprises: 5-97% acidic variant; and/or 5-97% basic variant; and/or 2-80% main isoform; and/or <35% oxidized variant; and/or <26% aggregated variant.

Fragmented variants (“fragment variant”) are variants which comprise a portion of a full length antibody. For example, such fragments include Fab, Fab′, F(ab′)2, and Fv fragments, diabodies, linear antibodies, single-chain antibody molecules and immunoglobulin single variable domains. The antibody composition may comprise <10% fragmented antibodies, such as <5%, <4.6%, <4.5%, <4.4%, <4.3%, <4.2%, <4.1%, <4%, <3.5%, <3%, <2.5%, <2%, <1.5%, <1%, <0.5% or <0.05% fragmented antibodies. In another embodiment, the composition may comprise 0.01-10%, 0.01-5%, 0.01-4.6%, 0.01-4.5%, 0.01-4%, 0.01-3.5%, 0.01-3%, 0.01-2.5%, 0.01-2%, 0.01-1.5%, 0.01-1%, 0.01-0.5%, 0.01-0.1%, or 0.01-0.05% fragmented antibodies. In another embodiment, the composition may comprise 0.5-10%, 0.5-5%, 0.5-4.6%, 0.5-4.5%, 0.5-4%, 0.5-3.5%, 0.5-3%, 0.5-2.5%, 0.5-2%, 0.5-1.5%, 0.5-1%, 0.6-1.5%, or 0.6-1.0% fragmented antibodies. Alternatively, the composition may comprise about 10%, about 5%, about 4%, about 3%, about 2%, about 1%, or about 0.5% fragmented antibodies. It will be understood that these fragmented variant embodiments may be combined with any one of the antibody variants described herein.

Deamidation, which may, for example, occur during production and/or storage, may be an enzymatic reaction or a chemical reaction. Deamidation may occur via simple chemical reaction through intramolecular cyclisation where the amide nitrogen of the next amino acid in the chain nucleophilicly attacks the amide (N+1 attacks N); forming a succinimide intermediate. Deamidation may primarily convert asparagine (N) to iso-aspartic acid (iso-aspartate) and aspartic acid (aspartate) (D) at an approximately 3:1 ratio. This deamidation reaction may therefore be related to isomerization of aspartate (D) to iso-aspartate. The deamidation of asparagine and the isomerization of aspartate, both may involve the intermediate succinimide. To a much lesser degree, deamidation can occur with glutamine residues in a similar manner. Deamidation can occur in a CDR, in a Fab (non-CDR region), or in an Fc region.

Isomerization is the conversion of aspartate (D) to iso-aspartate which involves the intermediate succinimide (succinimide-aspartic acid residue).

In one aspect, a composition comprises an antibody comprising a deamidation post-translational modification (“deamidation” or “deamidated”) also referred to herein as a “deamidated variant”.

In one embodiment, the antibody comprises deamidation of an asparagine residue in a CDR of the heavy chain sequence and/or a CDR of the light chain sequence. In a further embodiment, the antibody comprises deamidation of an asparagine residue in a CDR of the heavy chain sequence. In one embodiment, the antibody comprises deamidation of an asparagine residue in the Fc region of the heavy chain sequence and/or the Fc region of the light chain sequence. The deamidated variant may be present in one or both chains of the heavy chain or light chain. It will be understood that these deamidated variant embodiments may be combined with any one of the antibody variants described herein. In some embodiments, the deamidated variant comprises one or a combination of deamidation at: N30 of the CDRL1 and/or N388 and/or N393 of the Fc region of the heavy chain sequence.

In one embodiment, the deamidated variant comprises a deamidated residue selected from: an aspartic acid residue, a succinimide-aspartic acid residue, or an iso aspartic acid residue.

In one aspect, the composition comprises an antibody comprising a sequence that is at least about 90% identical to the light chain amino acid sequence of SEQ ID NO: 9 and optionally comprising a sequence that is at least about 90% identical to the heavy chain sequence of SEQ ID NO: 10, and comprises deamidation in the light chain sequence, for example, deamidation at amino acid residue N30 and/or N388 and/or N393 of the Fc heavy chain region. In some embodiments, the deamidated variant comprises up to 45% deamidation at N30 of SEQ ID NO: 1 or SEQ ID NO: 7, and/or N388 of SEQ ID NO: 10, and/or N393 of SEQ ID NO: 10.

Deamidation may result in a sequence change where an asparagine residue (N) is converted to an aspartic acid residue (D). Therefore, in one embodiment, the deamidated variant comprises a light chain sequence of SEQ ID NO: 11 (i.e., the CDRL1 sequence with N30D).

The composition may comprise up to 45% deamidated variant. In one aspect, the composition comprises an antibody having a heavy chain sequence of SEQ ID NO: 10 and a light chain sequence of SEQ ID NO: 9, wherein the composition comprises up to 45% deamidated variant.

In one embodiment, the composition comprises up to 45% deamidation at N30 of the CRDL1 and/or N388 and/or N393 of the heavy chain sequence. In one embodiment, the composition comprises 0-45%, 0-40%, 0-30%, 0-20%, or 0-10% deamidation at N30. Alternatively, the composition comprises 0.1-45%, 0.1-40%, 0.1-30%, 0.1-20%, or 0.1-10% deamidation at N30. Alternatively, the composition comprises 1-45%, 1-40%, 1-30%, 1-20%, or 1-10% deamidation at N30. Alternatively, the composition comprises 2-45%, 3-45%, 4-45%, 5-45%, 6-45%, 7-45%, 8-45%, 9-45%, 2-30%, 3-30%, 4-30%, 5-30%, 2-40%, 3-40%, 4-40%, 5-40%, 2-10%, 3-10%, 4-10%, or 5-9% deamidation at N30. Alternatively, the composition comprises 1% or more, 2% or more, 3% or more, 4% or more, 5% or more, 6% or more, 7% or more, 8% or more, 9% or more, or 10% or more deamidation at N30. As shown in the data presented herein, treated samples producing up to 34.8% deamidation at N30 gives 76% potency, therefore deamidation at N30 at levels higher than 34.8% would have a significant effect on relative potency (less than 76%).

In one embodiment, the composition comprises 0-45%, 0-40%, 0-30%, 0-20%, or 0-10% deamidation at N388. Alternatively, the composition comprises 0.1-45%, 0.1-40%, 0.1-30%, 0.1-20%, or 0.1-10% deamidation at N388.

Alternatively, the composition comprises 1-45%, 1-40%, 1-30%, 1-20%, or 1-10% deamidation at N388. Alternatively, the composition comprises 0.5% or more, 1% or more, or 2% or more deamidation at N388. As shown in the data presented herein, base treated samples producing up to 28.2% deamidation at N388 gives no effect on potency in bioassay (i.e., within assay variability—full function), therefore it is expected that deamidation at N388 can go higher than the reported level of 28.2% without any impact to relative potency or FcRn binding.

In one embodiment, the composition comprises 0-45%, 0-40%, 0-30%, 0-20%, or 0-10% deamidation at N393. Alternatively, the composition comprises 0.1-45%, 0.1-40%, 0.1-30%, 0.1-20%, or 0.1-10% deamidation at N393.

Alternatively, the composition comprises 1-45%, 1-40%, 1-30%, 1-20%, or 1-10% deamidation at N393. Alternatively, the composition comprises 0.5% or more, 1% or more, or 2% or more deamidation at N393. As shown in the data presented herein, base treated samples producing up to 28.2% deamidation at N393 gives no effect on potency in bioassay (i.e., within assay variability—full function), therefore it is expected that deamidation at N393 can go higher than the reported level of 28.2% without any impact to relative potency or FcRn binding.

In one aspect, the composition comprises an antibody having a heavy chain sequence of SEQ ID NO: 9 and a light chain sequence of SEQ ID NO: 10, wherein the composition comprises up to 45% deamidation at N388 and/or N393 of the heavy chain.

In one example, deamidation can be determined using Lys-C and/or tryptic peptide mapping tandem mass spectrometry (peptide mapping LC-MS/MS).

The antibody composition may comprise (i) the antibody (as described herein, e.g., an antibody comprising a heavy chain amino acid sequence of SEQ ID NO: 10 and a light chain amino acid sequence of SEQ ID NO: 11); and (ii) antibody variants that include one or more or a combination of: amino acid sequence variants (e.g., deamidated or C-terminal lysine clipped variants), oxidized variants, aggregated variants, and/or fragmented variants.

Therefore, in one aspect, there is provided a composition comprising an antibody having a heavy chain sequence of SEQ ID NO: 10 and a light chain sequence of SEQ ID NO: 9, wherein the composition comprises: (i)<35% oxidized variant; and (ii)<26% aggregated variant.

In one aspect, a composition comprises an antibody comprising a heavy chain sequence having one or a combination of sequences selected from SEQ ID NO: 10 and/or SEQ ID NO: 12 and a light chain sequence of SEQ ID NO: 9, wherein the composition comprises <35% oxidized variant.

In one aspect, a composition comprises an antibody comprising a heavy chain sequence having one or a combination of sequences selected from SEQ ID NO: 10 and/or SEQ ID NO: 12 and a light chain sequence of SEQ ID NO: 9, wherein the composition comprises, wherein the composition comprises <26% aggregated variant.

In another embodiment a composition comprising a variant has at least 60%, at least 65%, at least 70%, at least 75%, at least 80%, at least 85%, at least 90%, at least 95%, at least 96%, at least 97%, at least 98%, or at least 99% of the potency of the reference standard which has 100% potency. In one aspect, a composition comprises a variant of an anti-CTLA-4 antibody, wherein the variant comprises a heavy chain amino acid sequence comprising a CDRH1 of SEQ ID NO: 4, a CDRH2 of SEQ ID NO: 5, and a CDRH3 of SEQ ID NO: 6, and a light chain amino acid sequence comprising a CDRL1 of SEQ ID NO: 1, a CDRL2 of SEQ ID NO: 2, and a CDRL3 of SEQ ID NO: 3; wherein the composition has at least 60% of the potency of a composition comprising a heavy chain sequence of SEQ ID NO: 10 and a light chain sequence of SEQ ID NO: 9, 10-97% acidic variant, 10-97% basic variant, 2-80% main isoform, 30% or less HC W52 oxidized variant, 35% or less HC M256 oxidized variant, 12.5% or less HC M362 oxidized variant, 16% or less HC M401 oxidized variant, 35% or less HC M432 oxidized variant, 26% or less aggregated variant, 45% or less LC N30 deamidated variant, 45% or less HC N388 deamidated variant, 45% or less HC N393 deamidated variant, and/or 10% or less fragmented variants. Potency can be determined using the bioassay as described herein.

Glycation is a post-translational modification comprising a non-enzymatic chemical reaction between a reducing sugar, such as glucose, and a free amine group in the protein, and is typically observed at the epsilon amine of lysine side chains or at the N-terminus of the protein. Glycation can occur during production and/or storage in the presence of reducing sugars.

Disulfide bond scrambling can occur during production and/or storage conditions. Under certain circumstances, disulfide bonds may break or form incorrectly, resulting in unpaired cysteine residues (—SH). These free (unpaired) sulfhydryls (—SH) may promote shuffling.

The formation of a thioether and racemization of a disulfide bond can occur under basic conditions, in production or storage, through a beta elimination of disulfide bridges back to cysteine residues via a dehydroalanine and persulfide intermediate. Subsequent crosslinking of dehydroalanine and cysteine may result in the formation of a thioether bond or the free cysteine residues may reform a disulfide bond with a mixture of D- and L-cysteine.

Trisulfides may result from insertion of a sulfur atom into a disulfide bond (Cys-SS—S-Cys) and may be formed due to the presence of hydrogen sulfide in production cell culture.

N-terminal glutamine (Q, Gln) and glutamate (glutamic acid) (E, Glu) in the heavy chain and/or light chain may form pyroglutamate (pGlu) via cyclization. pGlu formation may form in the production bioreactor, but it can also be formed, for example, non-enzymatically, depending on pH and temperature of processing and storage conditions. Cyclization of N-terminal Q or E is commonly observed in natural human antibodies.

C-terminal lysine clipping (also referred to as C-terminal lysine cleavage) is an enzymatic reaction catalyzed by carboxypeptidases, and is commonly observed in recombinant and natural human antibodies. Variants of this process include removal of lysine from one or both heavy chains due to cellular enzymes from the recombinant host cell. Administration to the human subject/patient is likely to result in the removal of any remaining C-terminal lysine.

The binding of Neonatal Fc Receptor (FcRn) to an anti-CTLA-4 antibody can measured using surface plasmon resonance (SPR).

The antibody composition comprising the antibody and antibody variants described above retain specific antigen binding and/or FcRn binding and/or potency. For example, the antibody composition comprising the antibody and antibody variants and post-translational modification variants described above has >0.70 CTLA-4 specific antigen binding; and/or >70% FcRn binding and/or >70% potency. Thus, these levels (%) of variants can be tolerated in the antibody composition without significantly impacting function (i.e., without resulting in reduced activity). In one embodiment, “reduced function” or “reduced activity” means that binding to CTLA-4, or binding to FcRn, or potency is reduced as a percentage compared to a reference standard, and is significant over assay variability. For example, reduced function or activity or potency can be described as a reduction of >5%, >10%, >15%, >20%, >25%, >30%, >35%, >40%, >45%, or >50%.

The composition may comprise a mixture of antibody variants and post-translational modification variants. For example, the antibody composition may comprise two or more of acidic variants, basic variants, oxidation variants, deamidation variants, aggregated variants, and fragmented variants.

In one aspect, the composition comprises an antibody having a heavy chain sequence of SEQ ID NO: 10 and a light chain sequence of SEQ ID NO: 9, wherein the composition comprises any one or a combination of: (i) up to 100% acidic variant, (ii) up to 100% basic variant, (iii)<35% oxidation at W52 of the heavy chain, (iv)<35% oxidation at M256 of the heavy chain, (v)<12.5% oxidation at M362 of the heavy chain, (vi)<16% oxidation at M401, (vii)<35% oxidation at M432, and/or (viii)<26% aggregated variant.

In one aspect, the composition comprises an antibody having a heavy chain sequence of SEQ ID NO: 10 and a light chain sequence of SEQ ID NO: 9, wherein the composition comprises any one or a combination of: (i) up to 100% acidic variant, (ii) up to 100% basic variant, (iii)<35% oxidation at W52 of the heavy chain, (iv)<35% oxidation at M256 of the heavy chain, (v)<12.5% oxidation at M362 of the heavy chain, (vi)<160% oxidation at M401 of the heavy chain, (vii)<35% oxidation at M432 of the heavy chain, (viii)<26% aggregated variant, (ix)<45% deamidation at N30 of the light chain, (x)<45% deamidation at N388 of the heavy chain; (xi)<45% deamidation at N393 of the heavy chain, and/or (xii)<10% fragmented variants.

The present disclosure encompasses antibodies, which may have been subjected to, or have undergone, one or more of a post-translational modification described herein. Exemplary compositions may comprise a mixture or blend of antibodies: 1) with and without post-translational modifications (1 or more, or 2 or more) described herein. Therefore, the composition may comprise a population of antibodies with post-translational modifications and a population of antibodies without post-translational modifications.

The compositions described may have been subjected to, or have undergone, one or more post-translational modifications. The modification may occur in a CDR, the variable framework region, or the constant region. The modification may result in a change in charge of the molecule.

In one embodiment, a post-translational modification described herein, does not result in a significant change in antigen binding affinity, biological activity, pharmacokinetics (PK), aggregation, immunogenicity, and/or binding to an Fc receptor, except where specified and described as a product-related impurity.

### 4. Composition Impurities

Composition Impurities may include host cell proteins (HCPs). HCPs are process-related impurities that are generated by the host organism. HCP impurities can be reported as ng of HCP per mg of drug (ppm).

In one embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤13.54 ppm endoplasmic reticulum chaperone, BiP.

In another embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤5.21 ppm Elongation factor 1-gamma.

In another embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤3.41 ppm T-complex protein 1 subunit eta.

In another embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤3.24 ppm Importin subunit beta-1.

In another embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤2.75 ppm Glyceraldehyde-3-phosphate dehydrogenase.

In another embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤2.52 ppm RNA-splicing ligase RtcB homolog.

In another embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤1.53 ppm RuvB-like 2 OS=Mus musculus.

In another embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤1.46 ppm T-complex protein 1 subunit zeta.

In a further embodiment there is provided a composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; wherein the composition comprises ≤13.5 ppm endoplasmic reticulum chaperone, BiP, 5.21 ppm Elongation factor 1-gamma, ≤3.41 ppm T-complex protein 1 subunit eta, ≤3.24 ppm Importin subunit beta-1, <2.75 ppm Glyceraldehyde-3-phosphate dehydrogenase, ≤2.52 ppm RNA-splicing ligase RtcB homolog, ≤1.53 ppm RuvB-like 2 OS=Mus musculus and ≤1.46 ppm T-complex protein 1 subunit zeta.

### 5. Methods of Production

The antibodies and compositions described herein can be obtained by any means, including via in vitro sources (e.g., a hybridoma or a cell line producing an antibody recombinantly) and in vivo sources (e.g., rodents). Methods for generating antibodies are known in the art and are described, for example, in U.S. Pat. No. 6,682,736.

For example, the compositions may be expressed in and purified from recombinant expression systems. In one embodiment, the composition is produced by a method of culturing a host cell under conditions suitable for expression of an antibody comprising SEQ ID NO: 9 and SEQ ID NO: 10, wherein the composition is expressed, and optionally purified, and optionally formulated within a pharmaceutical composition.

A number of different expression systems and purification regimes can be used to produce the compositions. Generally, host cells are transformed with a recombinant expression vector encoding the antibody. A wide range of host cells can be employed, including eukaryotic cell lines of mammalian origin (e.g., CHO, Perc6, HEK293, HeLa, NS0).

The host cell may be an isolated host cell. The host cell is usually not part of a multicellular organism (e.g., plant or animal). The host cell may be a non-human host cell.

Appropriate cloning and expression vectors for use with eukaryotic or mammalian cellular hosts and methods of cloning are known in the art.

The host cells are cultured to express the recombinant expression vector encoding the antibody.

The composition may be recovered and purified by conventional protein purification procedures. For example, the composition may be harvested directly from the culture medium. Harvest of the cell culture medium may be via clarification, for example by centrifugation and/or depth filtration. Recovery of the composition is followed by purification to ensure adequate purity. Therefore, in one aspect, there is provided a cell culture medium comprising the composition described herein. In one embodiment, the cell culture medium comprises CHO cells.

The composition may be subsequently purified from the cell culture medium. This may comprise harvesting the cell culture supernatant, placing the cell culture supernatant in contact with a purification medium (e.g., protein A resin or protein G resin to bind antibody molecules) and eluting the antibody molecules from the purification medium to produce an eluate. Therefore, in one aspect, there is provided an eluate comprising the composition described herein.

One or more chromatography steps may be used in purification, for example one or more chromatography resins; and/or one or more filtration steps. For example affinity chromatography using resins, such as protein A, G, or L may be used to purify the composition. Alternatively, or in addition to, an ion-exchange resin such as a cation-exchange may be used to purify the composition.

Alternatively, the purification steps comprise: an affinity chromatography resin step, followed by a cation-exchange resin step.

### 6. Pharmaceutical Compositions and Formulations

A composition described herein can be in the form of a pharmaceutical composition.

In one aspect there is provided a pharmaceutical composition comprising the composition and at least one pharmaceutically acceptable excipient

A “pharmaceutical composition” may comprise a composition described herein (i.e., active ingredient), and one or more pharmaceutically acceptable excipients. The excipient(s) must be acceptable in the sense of being compatible with the other ingredients of the formulation, capable of pharmaceutical formulation, not deleterious to the recipient thereof, and/or do not interfere with the efficacy of the active ingredient. Therefore, pharmaceutical compositions of the disclosure are suitable for administration to a patient.

As used herein, “pharmaceutically acceptable excipient” may include one or more of buffering agents, water, saline, phosphate buffered saline, dextrose, glycerol, ethanol and the like, as well as combinations thereof. In many cases, it will be preferable to include isotonic agents, for example, polyol, sugars, polyalcohols such as mannitol, sorbitol, or sodium chloride, preservatives; co-solvents; antioxidants including ascorbic acid and methionine; chelating agents such as EDTA; metal complexes (e.g., Zn2+-protein complexes); biodegradable polymers; and/or salt-forming counter ions such as sodium or potassium.

The precise nature of the excipient or other material may depend on the route of administration, which may be, for example, oral, rectal, nasal, topical (including buccal and sublingual), vaginal, parenteral (including subcutaneous, intramuscular, intravenous, intradermal, intrathecal, and epidural), and intratumorally. It will be appreciated that the preferred excipient may vary with, for example, the condition of the recipient and the disease to be treated.

A mixture of excipients and concentrations of each together form a “pharmaceutical formulation” (or “formulation”). Such compositions are suitably free of visible particulate matter. The formulation may be in liquid form or lyophilized form. A composition in a liquid formulation may be filled into containers and frozen. In certain embodiments, aliquots of the frozen formulation comprising the composition may be lyophilized. Lyophilizate may be reconstituted by the addition of water or other aqueous solution to produce a reconstituted formulation comprising the composition.

In one embodiment, the composition is in a liquid formulation. In some embodiments, the formulation comprises the antibody at about 10 mg/mL to about 125 mg/mL. In a further embodiment, the formulation comprises the antibody at about 15 mg/mL to about 125 mg/mL, such as about 15 mg/mL to about 100 mg/mL, in particular about 15 mg/mL to about 50 mg/mL. In a further embodiment, the formulation comprises the antibody at about 20 mg/mL. In an alternative embodiment, the formulation comprises the antibody at about 50 mg/mL.

In one aspect there is provided a formulation comprising the pharmaceutical composition described herein comprising the antibody at about 10 mg/mL to about 125 mg/mL (such as about 20 mg/mL to about 50 mg/mL, e.g., 20 mg/mL) and a buffering agent at a pH of about 5.0 to about 6.5, e.g., a pH of 5.5. In one embodiment, the composition is in a liquid formulation.

In some embodiments, the composition is formulated as a sterile liquid. In some embodiments, the composition is free from visible particles. In some embodiments, the composition is formulated in a buffer (e.g., a histidine buffer). In some embodiments, the composition comprises a CTLA-4 antibody and two or more of the following: citrate buffer, histidine buffer, arginine, trehalose, sodium chloride and polysorbate 80. In certain embodiments, a buffering agent is a citrate buffer. Citrate buffer can be achieved, for example, by the use of a conjugate acid/conjugate base system (sodium citrate/citric acid) or by HCl titration of a sodium citrate solution. In one embodiment, the citrate buffer is at a pH of about 5.0 to about 6.5, such as about 5.5 to about 6.0, in particular about 5.5 or about 6.0.

In an alternative embodiment, the buffering agent is a histidine buffer. In one embodiment, the histidine buffer is at a pH of about 5.5 to about 7.0, about 5.5 to about 6.5, such as about 6.0 to about 6.5, in particular about 6.0 or about 6.5.

In some embodiments, a formulation comprises a surfactant. “Surfactants” are surface active agents that can exert their effect at surfaces of solid-solid, solid-liquid, liquid-liquid, and liquid-air interfaces because of their chemical composition, containing both hydrophilic and hydrophobic groups. Surfactants may reduce the concentration of proteins in dilute solutions at the air-water and/or water-solid interfaces where proteins can be adsorbed and potentially aggregated. Surfactants can bind to hydrophobic interfaces in protein formulations. Some parentally acceptable non-ionic surfactants comprise either polysorbate or polyether groups. Polysorbate 20 and 80, in particular polysorbate 80 (PS80), are suitable surfactant stabilizers in formulations of the disclosure. In one embodiment, the formulation additionally comprises PS80. In some embodiments, a formulation comprises PS80 or PS20 at about 0.01% to about 0.1%, such as about 0.01% to about 0.05% or about 0.01 to about 0.03% w/v. In some embodiments, the formulation comprises PS80 or PS20 at about 0.02% w/v. In a preferred embodiment, a formulation comprises PS80 at about 0.02% w/v.

The formulation may comprise a chelating agent. As used herein, the terms “chelating agent” generally refers to an excipient that can form at least one bond (e.g, covalent, ionic, or otherwise) to a metal ion. A chelating agent is typically a multidentate ligand that can be used in selected liquid compositions as a stabilizer to complex with species, which might promote instability.

Chelating agents that are suitable for use in the present invention, include, but are not limited to, aminopolycarboxylic acids, hydroxyaminocarboxylic acids, N-substituted glycines, 2-(2-amino-2-oxoethyl) aminoethane sulfonic acid (BES), deferoxamine (DEF), citric acid, niacinamide, and desoxy cholates. Examples of suitable aminopolycarboxylic acids include ethylenediaminetetraacetic acid (EDTA), diethylenetriamine pentaacetic acid 5 (DTPA), nitrilotri-acetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid. Examples of suitable hydroxyaminocarboxylic acids include N-hydroxy-ethyliminodiacetic acid (HIMDA), N,N-bis-hydroxyethyl-glycine (bicine) and N-(trishydroxymethylmethyl) 10 gly-cine (tricine). An example of a suitable N-substituted glycine is glycylglycine. An example of a suitable desoxycholate is sodium desoxycholate. Mixtures of two or more chelating agents are also encompassed by the present invention. In some embodiments the chelating agent is EDTA. In another embodiment, the chelating agent is disodium edetate dihydrate.

In some embodiments, the formulation comprises a polyol. In some embodiments, the polyol is a sugar, and preferably a non-reducing sugar. In some embodiments, the non-reducing sugar is trehalose. In some embodiments, the formulation comprises trehalose in the range from about from about 2% to about 10% w/v. In some embodiments, the formulation comprises about 5% w/v trehalose.

In some embodiments, the formulation comprises arginine and/or trehalose, such as arginine at about 80 mM to about 120 mM (in particular 100 mM) or trehalose at about 2% to about 10% w/v (in particular 5% w/v). In one embodiment the formulation comprises 222 mM trehalose dihyrate.

In one aspect, there is provided a formulation comprising the pharmaceutical composition, comprising the antibody at about 5 mg/mL to about 125 mg/mL, histidine buffer at about 10 mM to about 40 mM, trehalose dihydrate at about 200 to about 250 mM, disodium edetate dihydrate at about 0.2 mM to about 0.3 mM, and polysorbate 80 at about 0.01% to about 0.03% w/v, at a pH of about 5.0 to about 5.8.

In one aspect, there is provided a formulation comprising about 5-125 mg/mL of the antibody, about 20 mM histidine buffer, about 222 mM trehalose dihydrate, about 0.27 mM disodium edetate dihydrate and about 0.02% (w/v) polysorbate 80, at about pH 5.5.

In one aspect, there is provided a formulation comprising 20 mg/mL of the antibody, 20 mM histidine buffer, 222 mM trehalose dihydrate, 0.27 mM disodium edetate dihydrate and 0.02% (w/v) polysorbate 80, at pH 5.5.

A “stable” formulation is one in which the protein therein essentially retains its physical and/or chemical stability during manufacturing, transport, storage, and administration. Stability can be measured by differential scanning calorimetry, where higher temperatures of transition are a more stable product formulation. A “stable” formulation may be one wherein, the phase transition does not occur until about 50° C. or more, about 55° C. or more, about 60° C. or more. Stability can be measured at a selected temperature for a selected time period. For example, for a product stored at a recommended temperature of 2° C. to 8° C., the formulation is stable at room temperature, about 30° C., or at 40° C., for at least 1 month and/or stable at about 2 to 8° C. for at least 1 year and preferably for at least 2 years. For example, the extent of aggregation, acidic variant, and/or basic variant during storage can be used as an indicator of protein stability. Thus, a “stable” formulation may be one wherein, about 10% or less, about 5% or less, for example about 4% or less aggregation variant of the antibody is present in the formulation. A “stable” formulation may be one wherein, about 60% or less, about 50% or less, for example about 45% or less acidic variant of the antibody is present in the formulation. A “stable” formulation may be one wherein, about 35% or less, about 10% or less, for example about 20% or less basic variant of the antibody is present in the formulation.

In certain aspects of the disclosure, a formulation allows the composition to remain stable to storage at about 2 to 8° C. for at least 18 months, freezing, thawing, and/or mixing. In one embodiment, a “stable” formulation may be one wherein, about 4% or less aggregation variant, about 45% or less acidic variant, and about 20% or less basic variant of the antibody is present in the formulation.

In yet another aspect, the present disclosure relates to an article of manufacture, form examples, a kit, comprising a container holding a composition in a formulation described herein. In one aspect there is provided an injection device comprising the formulation. The injection device may comprise a pen injector device or an autoinjector device. In one embodiment, the formulation is contained in a prefilled syringe.

### 7. Methods of Treatment and Compositions for Use

The present disclosure further provides a method of treating any disease or disorder in which the improper expression (e.g., overexpression) or increased activity of a CTLA-4 protein causes or contributes to the pathological effects of the disease, or a decrease in CTLA-4 protein levels or activity has a therapeutic benefit in mammals, preferably humans.

In one aspect, there is provided a composition described herein for use in therapy. Such therapy may relate to any disease or disorder in which the improper expression (e.g., overexpression) or increased activity of a CTLA-4 protein causes or contributes to the pathological effects of the disease, or a decrease in CTLA-4 protein levels or activity has a therapeutic benefit in mammals, preferably humans.

The compositions may be used in methods of increasing T cell activation or T cell effector function in a subject, which method comprises administering a therapeutically effective dose of an agent that is capable of inhibiting CTLA-4 signaling. The compositions may be used in methods of inducing an immune response in a subject, which method comprises administering a therapeutically effective dose of an agent that is capable of inhibiting CTLA-4 signaling. The compositions may be used in methods of enhancing an immune response or increasing the activity of an immune cell in a subject, which method comprises administering a therapeutically effective dose of an agent that is capable of inhibiting CTLA-4 signaling.

In one aspect, there is provided a composition for use in the treatment of cancer.

In one aspect, there is provided use of a composition described herein in the manufacture of a medicament for use in the treatment of cancer.

By the term “treating” and grammatical variations thereof as used herein, is meant therapeutic therapy. In reference to a particular condition, treating means: (1) to ameliorate the condition of one or more of the biological manifestations of the condition, (2) to interfere with a) one or more points in the biological cascade that leads to or is responsible for the condition or b) one or more of the biological manifestations of the condition, (3) to alleviate one or more of the symptoms, effects or side effects associated with the condition or treatment thereof, (4) to slow the progression of the condition or one or more of the biological manifestations of the condition or (5) to prevent the onset of one or more of the biological manifestations of the condition.

Treatment can be therapeutic, prophylactic or preventative. The subject will be one who is in need thereof. Those in need of treatment may include individuals already suffering from a particular medical disease, in addition to those who may develop the disease in the future.

Therefore, prophylactic therapy is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

Thus, the methods, antibodies and compositions described herein can be used for prophylactic treatment or preventative treatment if specified. In this case, the described methods, antibodies and compositions can be used to prevent or delay the onset of one or more aspects or symptoms of a disease. The subject can be asymptomatic. The subject may have a genetic predisposition to the disease. A prophylactically effective amount of the composition is administered to such an individual. A prophylactically effective amount is an amount which prevents or delays the onset of one or more aspects or symptoms of a disease described herein.

The methods, antibodies and compositions need not affect a complete cure, or eradicate every symptom or manifestation of the disease to constitute a viable therapeutic treatment. As is recognized in the art, drugs employed as therapeutic agents in methods of treatment may reduce the severity of a given disease state, but need not abolish every manifestation of the disease to be regarded as useful therapeutic agents. Similarly, a prophylactically administered treatment need not be completely effective in preventing the onset of a disease in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur (for example by delaying the onset of the disease) or worsen in a subject, is sufficient.

The terms “individual,” “subject,” and “patient” are used herein interchangeably and may be is defined broadly to include any person in need of treatment, for example, a person in need of cancer treatment. The subject is typically a human. The subject may also be a mammal, such as a mouse, rat, or primate (e.g., a marmoset or monkey). The subject can be a non-human animal. The antibodies, compositions and methods of the disclosure also have veterinary use. The subject to be treated may be a farm animal, for example, a cow or bull, sheep, pig, ox, goat or horse, or may be a domestic animal such as a dog or cat. The animal may be any age, or a mature adult animal.

The disclosure also provides a method of treating a cancer in a mammal.

### 8. Cancer Treatment

The present disclosure provides methods of reducing tumors or inhibiting the growth of tumor cells in a subject, which method comprises administering a therapeutically effective dose of an agent that is capable of inhibiting CTLA-4 signaling. The method may comprise administering the aforementioned composition to a mammal having a cancer, whereupon the cancer is treated in the mammal. As discussed herein, CTLA-4 is abnormally expressed in a variety of cancers and CTLA-4 expression in some cancers (e.g., renal cell carcinoma) patients correlates with tumor aggressiveness. The method can be used to treat any type of cancer known in the art, such as, for example, adenocarcinoma, adenocarcinoma of the lung, acute myeloid leukemia (“AML”), acute lymphoblastic leukemia (“ALL”), adrenocortical carcinoma, anal cancer, appendiceal cancer, B-cell derived leukemia, B-cell derived lymphoma, bladder cancer, brain cancer, breast cancer (e.g., triple negative breast cancer (TNBC)), cancer of the fallopian tube(s), cancer of the testes, cerebral cancer, cervical cancer, choriocarcinoma, chronic myelogenous leukemia, a CNS tumor, colon adenocarcinoma, colon cancer, colorectal cancer, diffuse intrinsic pontine glioma (DIPG), diffuse large B cell lymphoma (“DLBCL”), embryonal rhabdomyosarcoma (ERMS), endometrial cancer, epithelial cancer, esophageal cancer, Ewing's sarcoma, follicular lymphoma (“FL”), gall bladder cancer, gastric cancer, gastrointestinal cancer, glioma, head and neck cancer, a hematological cancer, hepatocellular cancer, Hodgkin's lymphoma/primary mediastinal B-cell lymphoma, kidney cancer, kidney clear cell cancer, laryngeal cancer, leukemia, liver cancer, lung cancer, lymphoma, melanoma, Merkel cell carcinoma, mesothelioma, monocytic leukemia, multiple myeloma, myeloma, a neuroblasts-derived CNS tumor, non-Hodgkin's lymphoma (NHL), non-small cell lung cancer (NSCLC), oral cancer, osteosarcoma, ovarian cancer, ovarian carcinoma, pancreatic cancer, peritoneal cancer, primary peritoneal cancer, prostate cancer, relapsed or refractory classic Hodgkin's Lymphoma (cHL), renal cell carcinoma, rectal cancer, salivary gland cancer (e.g., a salivary gland tumor), sarcoma, skin cancer, small cell lung cancer, small intestine cancer, squamous cell carcinoma of the anogenital region (e.g., squamous cell carcinoma of the anus, penis, cervix, vagina, or vulva), squamous cell carcinoma of the esophagus, squamous cell carcinoma of the head and neck (SCHNC), squamous cell carcinoma of the lung, stomach cancer, T-cell derived leukemia, T-cell derived lymphoma, thymic cancer, a thymoma, thyroid cancer, uveal melanoma, urothelial cell carcinoma, uterine cancer, uterine endometrial cancer, uterine sarcoma, vaginal cancer, vulvar cancer, or Wilms tumor. In one embodiment the cancer is hepatocellular carcinoma. In another embodiment the cancer is NSCLC.

In some embodiments, a cancer to be treated with the compositions described herein is characterized by microsatellite instability or lack thereof. Microsatellite instability (“MSI”) is or comprises a change that in the DNA of certain cells (such as tumor cells) in which the number of repeats of microsatellites (short, repeated sequences of DNA) is different than the number of repeats that was contained in the DNA from which it was inherited. Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load.

In some embodiments, a cancer has a microsatellite instability status of high microsatellite instability (e.g., MSI-H status). In some embodiments, a cancer has a microsatellite instability status of low microsatellite instability (e.g., MSI-L status). In some embodiments, a cancer has a microsatellite instability status of microsatellite stable (e.g., MSS status). In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In embodiments, the cancer is associated with a high tumor mutation burden (TMB). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS.

In some embodiments, a cancer is a mismatch repair deficient (dMMR) cancer. Microsatellite instability may arise from a failure to repair replication-associated errors due to a defective DNA mismatch repair (MMR) system. This failure allows persistence of mismatch mutations all over the genome, but especially in regions of repetitive DNA known as microsatellites, leading to increased mutational load that may improve responses to certain anti-CTLA-4 agents.

In some embodiments, a cancer is a hypermutated cancer. In some embodiments, a cancer harbors a mutation in polymerase epsilon (POLE). In some embodiments, a cancer harbors a mutation in polymerase delta (POLD).

In some embodiments, a cancer is endometrial cancer (e.g., MSI-H or MSS/MSI-L endometrial cancer). In some embodiments, a cancer is a MSI-H cancer comprising a mutation in POLE or POLD (e.g., a MSI-H non-endometrial cancer comprising a mutation in POLE or POLD).

In one aspect, there is provided a method of treating cancer comprising administering to a subject in need thereof a therapeutically effective amount of a composition (including a pharmaceutical composition or formulation) described herein.

As used herein, the terms “cancer,” and “tumor” are used interchangeably and, in either the singular or plural form, refer to cells that have undergone a transformation, such as malignant transformation, that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

In embodiments, a cancer that is a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, an ovarian cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus).

In some embodiments, the cancer is a hematological cancer. In some embodiments, the hematological cancer is selected from: Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), acute lymphoblastic leukemia (“ALL”), or Multiple myeloma (“MM”). In embodiments, a cancer is a blood-borne cancer such as acute lymphoblastic leukemia (“ALL”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute non-lymphocytic leukemia, acute undifferentiated leukemia, chronic myelocytic leukemia (“CML”), chronic lymphocytic leukemia (“CLL”), hairy cell leukemia and multiple myeloma; acute and chronic leukaemias such as lymphoblastic, myelogenous, lymphocytic, and myelocytic leukaemias.

In some embodiments, the cancer is a lymphoma such as Hodgkin's disease, non-Hodgkin's Lymphoma, Multiple myeloma, Waldenstrom's macroglobulinemia, Heavy chain disease and Polycythemia vera.

In some embodiments, the cancer is a squamous cell carcinoma. In some embodiments, the cancer is squamous cell carcinoma of the lung. In some embodiments, the cancer is squamous cell carcinoma of the esophagus. In some embodiments, the cancer is head and neck squamous cell carcinoma (HNSCC). In some embodiments, the cancer is squamous cell carcinoma of the anogenital region (e.g., of the anus, penis, cervix, vagina, or vulva).

In some embodiments, the cancer is bladder cancer, breast cancer (e.g., triple negative breast cancer (TNBC)), cancer of the fallopian tube(s), cholagiocarcinoma, colon adenocarcinoma, endometrial cancer, esophageal cancer, Ewing's sarcoma, gastric cancer, kidney clear cell cancer, lung cancer (e.g., lung adenocarcinoma or lung squamous cell cancer), mesothelioma, ovarian cancer, pancreatic cancer, peritoneal cancer, prostate cancer, uterine endometrial cancer, or uveal melanoma. In some embodiments, the cancer is ovarian cancer, cancer of the fallopian tube(s), or peritoneal cancer. In some embodiments, the cancer is breast cancer (e.g., TNBC). In some embodiments, the cancer is lung cancer (e.g., non-small cell lung cancer). In some embodiments, the cancer is prostate cancer.

In some embodiments, the cancer is a CNS or brain cancer such as neuroblastoma (NB), glioma, diffuse intrinsic pontine glioma (DIPG), pilocytic astrocytoma, astrocytoma, anaplastic astrocytoma, glioblastoma multiforme, medulloblastoma, craniopharyngioma, ependymoma, pinealoma, hemangioblastoma, acoustic neuroma, oligodendroglioma, meningioma, vestibular schwannoma, adenoma, metastatic brain tumor, meningioma, spinal tumor, or medulloblastoma. In embodiments, a cancer is a CNS tumor.

In some embodiments, the cancer is a solid tumor. In embodiments, a cancer is a solid tumor such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary

carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma. In some embodiments, the tumor is an advanced stage solid tumor. In some embodiments, the tumor is a metastatic solid tumor.

In some embodiments, the cancer is a gynecologic cancer (i.e., a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In some embodiments, the cancer is ovarian cancer (e.g., serous or clear cell ovarian cancer). In some embodiments, the cancer is fallopian tube cancer (e.g., serous or clear cell fallopian tube cancer). In some embodiments, the cancer is primary peritoneal cancer (e.g., serous or clear cell primary peritoneal cancer).

In some embodiments, the ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer develops in the egg-producing cells of the ovaries. In some embodiments, the cancer is or comprises a stromal tumor. Stromal tumors develop in the connective tissue cells that hold the ovaries together, which sometimes is the tissue that makes female hormones called estrogen. In some embodiments, the cancer is or comprises a granulosa cell tumor. Granulosa cell tumors may secrete estrogen resulting in unusual vaginal bleeding at the time of diagnosis. In some embodiments, the gynecologic cancer is associated with homologous recombination repair deficiency/homologous repair deficiency (“HRD”) and/or BRCA1/2 mutation(s). In some embodiments, the gynecologic cancer is platinum-sensitive. In some embodiments, the gynecologic cancer has responded to a platinum-based therapy. In some embodiments, the gynecologic cancer has developed resistance to a platinum-based therapy. In some embodiments, the gynecologic cancer has at one time shown a partial or complete response to platinum-based therapy (e.g., a partial or complete response to the last platinum-based therapy or to the penultimate platinum-based therapy). In some embodiments, the gynecologic cancer is now resistant to platinum-based therapy.

In some embodiments, the cancer is a breast cancer. Usually breast cancer either begins in the cells of the milk producing glands, known as the lobules, or in the ducts. Less commonly, breast cancer can begin in the stromal tissues. These include the fatty and fibrous connective tissues of the breast. Over time, the breast cancer cells can invade nearby tissues such the underarm lymph nodes or the lungs in a process known as metastasis. The stage of a breast cancer, the size of the tumor and its rate of growth are all factors which determine the type of treatment that is offered.

Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative.

In some embodiments, the cancer is ER-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or TNBC. In embodiments, a cancer is TNBC.

In some embodiments, the breast cancer is a metastatic breast cancer. In some embodiments, the breast cancer is an advanced breast cancer. In some embodiments, the cancer is a stage II, stage III or stage IV breast cancer. In some embodiments, the cancer is a stage IV breast cancer.

In some embodiments, the cancer is endometrial cancer (“EC”). In some embodiments, the endometrial cancer is metastatic endometrial cancer.

Endometrial carcinoma is the most common cancer of the female genital tract. The annual number of new cases of endometrial cancer (EC) is estimated at about 325,000 worldwide. Further, EC is the most commonly occurring cancer in postmenopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in first line (1 L) and second line (2L) therapy settings. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC) while type II are non-endometrioid (NEEC) (mainly serous and clear cell) carcinomas. The World Health Organization has recently updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, low-grade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyper-estrogenism. At the microscope, SC shows thick, fibrotic or edematous papillae with prominent stratification of tumor cells, cellular budding, and anaplastic cells with large, eosinophilic cytoplasms. The vast majority of EEC are low grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively.

In some embodiments, the cancer is a lung cancer. In some embodiments, the lung cancer is a squamous cell carcinoma of the lung. In some embodiments, the lung cancer is small cell lung cancer (SCLC). In some embodiments, the lung cancer is non-small cell lung cancer (NSCLC) such as squamous NSCLC. In some embodiments, the lung cancer is an ALK-translocated lung cancer (e.g., ALK-translocated NSCLC). In some embodiments, the lung cancer is an EGFR-mutant lung cancer (e.g., EGFR-mutant NSCLC).

In some embodiments, the cancer is a metastatic cancer.

In some embodiments, the cancer is a recurrent cancer (e.g., a recurrent gynecological cancer such as recurrent epithelial ovarian cancer, recurrent fallopian tube cancer, recurrent primary peritoneal cancer, or recurrent endometrial cancer).

The subject in need of cancer treatment may include patients from a variety of stages including newly diagnosed, relapsed, refractory, progressive disease, remission, and others. The subject in need of cancer treatment may also include patients who have undergone stem cell transplant or who are considered transplant ineligible.

### 9. Routes of Administration

Subjects may have had at least one prior cancer therapy before being treated with the compositions of the present disclosure. In one embodiment, the subject has been treated with at least 1, at least 2, at least 3, at least 4, at least 5, at least 6, or at least 7 prior cancer therapies before being treated with the compositions of the present disclosure. In another embodiment, the subject has newly diagnosed cancer and has had 0 prior therapies before being treated with the compositions of the present disclosure.

The compositions of the disclosure may be administered by any appropriate route. For some compositions, suitable routes include oral, rectal, nasal, topical (including buccal and sublingual), vaginal, parenteral (including subcutaneous, intramuscular, intravenous, intradermal, intrathecal, and epidural), and intratumorally.

It will be appreciated that the preferred route may vary with, for example, the condition of the recipient and the cancer to be treated.

In some embodiments, the composition is administered intravenously (e.g., by intravenous (IV) infusion). In a further embodiment, the composition is administered through a 30 minute IV infusion.

In some embodiments, the composition is administered by injection. Therefore, in one aspect there is provided an injection device comprising the composition, pharmaceutical composition or formulation of the disclosure. The injection device may comprise a pen injector device or an autoinjector device.

In one embodiment, the composition is contained in a prefilled syringe.

The desired dosage can be delivered by a single bolus administration of the composition, by multiple bolus administrations of the composition, or by continuous infusion administration of the composition.

In certain embodiments, a composition of the disclosure is administered as a pharmaceutical composition.

The term “administering” as used herein is meant to refer to the delivery of the compositions described herein to achieve a therapeutic objective. The compositions may be administered at an administration interval for a period sufficient to achieve clinical benefit.

The composition may be administered to the subject in such a way as to target therapy to a particular site.

In certain embodiments, a composition can be co-administered to a subject with one or more additional therapeutic agents. In another embodiment, a composition can be co-administered to a subject with one or more additional cancer therapeutics. The additional cancer therapeutic agent may include, but is not limited to, other immunomodulatory drugs, therapeutic antibodies, CAR-T therapeutics, BiTEs, HDAC inhibitors, proteasome inhibitors, anti-inflammatory compounds, and immunomodulatory imide drugs (IMiD).

“Co-administered” means the administration of two or more different pharmaceutical compositions or treatments (e.g., radiation treatment) that are administered to a subject by combination in the same pharmaceutical composition or separate pharmaceutical compositions. Thus, co-administration involves administration at the same time of a single pharmaceutical composition comprising two or more pharmaceutical agents or administration of two or more different compositions to the same subject at the same or different times.

In some embodiments, the composition can be administered in combination with other agents that inhibit immune checkpoint pathways. For example, the composition can be administered in combination with agents that inhibit or antagonize the PD-1 pathway.

In some embodiments, the method comprises administering the composition in combination with durvalumab. In some embodiments, the combination is for administration to patients with a liver cancer (hepatocellular carcinoma), an ovarian cancer, a head and neck cancer, a lung cancer (e.g., a non-small cell lung cancer (NSCLC)), a renal cancer, a bladder cancer, a melanoma, Merkel cell carcinoma, a cervical cancer, a vaginal cancer, a vulvar cancer, a uterine cancer, a endometrial cancer, a fallopian tube cancer, a breast cancer, a prostate cancer, a salivary gland tumor, a thymoma, a adrenocortical carcinoma, a esophageal cancer, a gastric cancer, a colorectal cancer, an appendiceal cancer, a urothelial cell carcinoma, or a squamous cell carcinoma (e.g., of the lung; of the anogenital region including anus, penis, cervix, vagina, or vulva; or of the esophagus). In further embodiments, the cancer is selected from liver cancer, ovarian cancer or lung cancer (e.g., NSCLC).

### 10. Dosage

The compositions described herein may be administered in a therapeutically effective amount.

The term “therapeutically effective amount” or “therapeutically effective dose” of a composition as used herein refers to an amount of an agent (such as an antibody or a pharmaceutical composition) which provides a therapeutic benefit in the treatment or management of one or more symptoms of a condition to be treated.

Therapeutically effective amounts and treatment regimes are generally determined empirically and may be dependent on factors, such as the age, weight, and health status of the patient and disease or disorder to be treated. Such factors are within the purview of the attending physician.

Ranges provided herein, of any type, include all values within a particular range described and values about an endpoint for a particular range.

In some embodiments, a therapeutically effective dose is a flat dose of about 10-500 mg (e.g., a flat dose about 10 mg, a flat dose about 25 mg, a flat dose about 50 mg, a flat dose about 75 mg, a flat dose about 100 mg, a flat dose about 200 mg, a flat dose about 300 mg, a flat dose about 400 mg, or a flat dose about 500 mg). In some embodiments, a therapeutically effective dose is about 1 mg/kg. In some embodiments, a therapeutically effective dose is about 3 mg/kg. In some embodiments, a therapeutically effective dose is about 10 mg/kg. In some embodiments, a therapeutically effective dose is about 15 mg/kg. In some embodiments, a therapeutically effective dose is a flat dose about 50 mg. In some embodiments, a therapeutically effective dose is about 75 mg. In some embodiments, a therapeutically effective dose is about 100 mg.

In one embodiment, the composition is administered once every 2-6 weeks (e.g., 2, 3, or 4 weeks, in particular 3 weeks).

In one embodiment, the composition is administered for once every 3 weeks for 2-6 dosing cycles (e.g., the first 3, 4, or 5 dosing cycles, in particular, the first 4 dosing cycles).

In some embodiments, the first dose and second dose are different. In some embodiments, the first dose is about 75 mg and the second dose is about 100 mg.

In some embodiments, the composition is administered at an administration interval (or treatment cycle) of once every 3 weeks (Q3W), once every 4 weeks (Q4W), once every 5 weeks (Q5W), or once every 6 weeks (Q6W). In some embodiments, the composition is administered at an administration interval (or treatment cycle) of once every three weeks (Q3W). In some embodiments, the composition is administered at an administration interval (or treatment cycle) of once every 4 weeks (Q4W). In some embodiments, the composition is administered at an administration interval (or treatment cycle) of once every 5 weeks (Q5W). In some embodiments, the composition is administered at an administration interval (or treatment cycle) of once every 6 weeks (Q6W). In some embodiments, the composition is administered for a period of at least about 2, 3, 4, 5, 6, 7, 8, 9, 10, 11, 12, 13, 14, 15, 16, 17, 18, 19, 20 weeks, or more. In some embodiments, the composition is administered on the first day of a treatment cycle or within 1, 2, or 3 days of the first day of a treatment cycle.

In some embodiments, the composition is administered at a dose of 75 mg, every 3 weeks. In another embodiment, the composition is administered at a dose of 75 mg, every 3 weeks for 4 cycles. In another embodiment, the composition is administered at a dose of 75 mg, every 3 weeks for 4 cycles followed by a further 75 mg dose at week 16.

In some embodiments, the composition is administered as a single dose of 300 mg. In another embodiment, the composition is administered as a single dose of 300 mg on Day 1 of the treatment cycle, in combination with 1500 mg durvalumab, where durvalumab monotherapy is continued every 4 weeks.

In some embodiments the compositions are administered according to Table 1.

TABLE 1
Recommended Dosage

| Indication | Recommended Dosage | Duration of Therapy |
|---|---|---|
| Metastatic | Duration chemotherapy: | Up to a maximum of 5 |
| NSCLC | 75 mg^{a, b} in combination with durvalumab | doses unless there was |
| | 1500 mg^{c} and platinum-based chemotherapy | disease progression or |
| | every 3 weeks (21 days) for 4 cycles | unacceptable toxicity |
| | Post-platinum chemotherapy: Durvalumab | |
| | 1500 mg^{b, c} every 4 weeks as monotherapy and | |
| | histology-based pemetrexed maintenance^{d, e} | |
| | therapy every 4 weeks and, | |
| | a fifth dose of 75 mg^{f, g} alongside durvalumab | |
| | dose 6 at week 16 | |
| uHCC | Single Tremelimumab Regular Interval | As long as clinical benefit |
| | Durvalumab (STRIDE): 300 mg^{h} as a single | is observed or until |
| | priming dose in combination with durvalumab | unacceptable toxicity |
| | 1500 mg^{h, i} at Cycle 1/Day 1, followed by | |
| | durvalumab monotherapy every 4 weeks | |

^{a}Patients with a body weight of 40 kg or less must receive weight-based dosing equivalent to Tremelimumab 1 mg/kg and durvalumab 20 mg/kg until the weight improves to greater than 30 kg.
^{b}Administer Tremelimumab first; followed by durvalumab and then chemotherapy on the day of dosing, when applicable
^{c}When Tremelimumab is administered in combination with durvalumab and chemotherapy, refer to the Prescribing Information for durvalumab for dosing information.
^{d}When Tremelimumab is administered in combination with durvalumab and platinum-based chemotherapy, refer to the Prescribing Information for nab-paclitaxel, gemcitabine, pemetrexed and carboplatin or cisplatin for dosing information.

In some embodiments, the composition described herein is administered according to dosing regimens demonstrated to achieve a clinical benefit the patient. In some embodiments, a clinical benefit is stable disease (“SD”), a partial response (“PR”) and/or a complete response (“CR”). In some embodiments, a clinical benefit is stable disease (“SD”). In embodiments, a clinical benefit is a partial response (“PR”).

In embodiments, a clinical benefit is a complete response (“CR”). In some embodiments, PR or CR is determined in accordance with Response Evaluation Criteria in Solid Tumors (RECIST). In some embodiments, the composition is administered for a longer period to maintain clinical benefit.

All patent and literature references disclosed herein are expressly and entirely incorporated herein by reference.

### EXAMPLES

### Example 1: Tremelimumab Impurites

Tremelimumab was assessed to determine product-related impurities and process-related impurities. Test methods used to evaluate impurities are described herein.

### Methods

### Amino Acid Analysis

Amino acid analysis was used to determine the amino acid composition of the protein. The samples were acid hydrolyzed, labelled and analyzed by liquid chromatography. Quantitation of each amino acid was determined relative to an external standard curve.

### Analytical Ultracentrifugation

Analytical Ultracentrifugation was used to measure purity, fragmentation, and aggregation. Species of different sizes were separated based on their sedimentation behaviour under a strong centrifugal field. Species resolved at sedimentation coefficients lower than the monomer peak were recorded as fragments. Species resolved at sedimentation coefficients higher than the monomer peak were recorded as aggregates.

### Competitive Binding ELISA

A competitive enzyme-linked immunosorbent assay (ELISA) was used as a binding assay. The assay measures the ability of tremelimumab to block the binding of CTLA-4 to B7.2 fusion protein. Binding activity was evaluated as a dose response normalized to Reference Standard and yielding a potency value relative to the tremelimumab Reference Standard.

Capillary Isoelectric Focusing (cIEF)

The charge heterogeneity of tremelimumab was determined by imaged capillary electrophoresis (iCE). Tremelimumab samples were mixed with carrier ampholytes and injected into a capillary. Separation of charge isoforms was achieved when an electric potential was applied and the carrier ampholytes formed a pH gradient within the capillary; tremelimumab charge isoforms focused at points of neutral net charge (pI) within the capillary.

### Reducing Capillary Gel Electrophoresis

Proteins were subjected to a denaturant, sodium dodecyl sulfate (SDS), under reducing conditions. When placed in an electric field in a capillary the SDS coated proteins migrate through a replaceable SDS polymer sieving matrix toward the anode, separating according to apparent molecular weight.

### Non-reducing Capillary Gel Electrophoresis

In non-reducing capillary gel electrophoresis, proteins were subjected to a denaturant, SDS, under non-reducing conditions. When placed in an electric field in a capillary, the SDS-coated proteins migrate through a replaceable SDS polymer sieving matrix toward the anode separating according to apparent molecular weight.

### FcRn Binding Assay by SPR

A surface plasmon resonance binding assay was used to measure FcRn binding. This method is based on the ability of the test sample to bind to recombinant FcRn that has been immobilized on an SPR sensor surface. A reference flow cell was created using the same standard amine coupling procedure without the addition of FcRn.

### High Performance Size Exclusion Chromatography

The purity (monomer) and product related impurity (aggregates) of tremelimumab were determined by high performance size exclusion chromatography (HPSEC).

The HPSEC method separates size variants according to their relative molecular size and shape using differential exclusion from stationary phase pores in a column. As the mobile phase carries the sample through the column, solutes diffuse into pores at rates inversely proportional to their hydrodynamic volume based on the solute size and pore exclusion limit. Separated variants were detected and quantified by a UV detector.

### Ion Exchange Chromatography (IEC)

Ion exchange chromatography was used to evaluate charge heterogeneity. Samples were injected onto an ion-exchange column and eluted with a salt gradient. Eluted protein was detected using UV absorbance at 220 (or 280) nm. Results were reported as percentage of 0-Lys, 1-Lys, and 2-Lys peaks. To evaluate charge heterogeneity in the absence of C-terminal lysine, samples were digested with Carboxypeptidase B (CBP) prior to analysis. Results were reported as area percentage of pre-peaks, main peak, and post-peaks.

### Ligand Binding Assay by SPR

This assay was performed using a surface plasmon resonance (SPR) to measure the association (ka) and dissociation (kd) for the interaction between tremelimumab and CTLA-4 The ratio of kd/ka provides information about the equilibrium dissociation constant (K_{D}) for the interaction between tremelimumab and CTLA-4. Binding affinity of test sample was calculated relative to the K_{D} of Reference Standard.

### Methionine Oxidation by Lys-C Mapping

Methionine oxidation of tremelimumab samples was quantitatively determined by peptide mapping. The resulting peptide fragments were separated by reversed-phase HPLC and detected using an ultraviolet detector. One methionine-containing peptide fragment and its respective oxidized form were monitored. Percent oxidation was reported for this methionine-256-containing fragment.

### N-terminal Sequencing

N-terminal sequencing by Edman degradation was used for determination of the amino acid sequence on the N-termini of heavy and light chains of tremelimumab. The results were compared to theoretical sequences of the light and heavy chains.

Peptide Mapping with Mass Spectrometry

Peptide mapping was used to verify primary sequences and evaluate post-translational modifications. Samples were denatured and reduced, alkylated and digested with trypsin. The peptides were separated by reverse phase liquid chromatography and were measured using a UV detector and a mass spectrometer. Results were reported with respect to consistency of the peptide fragmentation pattern of the test sample compared to Reference Standard.

### Q-TOF Mass Spectrometry

Q-TOF mass spectrometry (MS) was used to confirm the primary structure of the molecule based on differences of their mass to charge ratios (m/z). The measured mass of the molecule was obtained through the deconvolution of the m/z data and compared to the theoretical mass of the molecule based on the amino acid composition. Results were reported in units of molecular mass (Da).

### Reporter Gene Bioassay

CTLA4 is a cell surface receptor on activated T cells. The reporter gene bioassay measures the ability (potency) of tremelimumab to attenuate CTLA4-mediated inhibitory signals during T cell activation by blocking CTLA4 from binding to the natural ligands, B7.1 (CD80) and B7.2 (CD86) on the antigen presenting cells. Tremelimumab was incubated at varying concentrations with the cell lines in the presence of Super Antigen SEE. The amount of luminescence generated is proportional to the T cell activity and is quantified in a luminometer after reaction with luciferase substrate. The potency of the test sample was determined relative to Reference Standard.

Reversed-Phase HPLC with Mass Spectrometry

RP-HPLC/MS method was used only for peak identification. Samples were deglycosylated using PNGase F. Both non-reduced and reduced samples were separated by RP-HPLC and analyzed by Q-TOF mass spectrometer Mass spectrometric data were collected and analysed. Each peak in the RP-HPLC profile was identified and reported.

### Product-Related Impurities

Product-related impurities such as aggregates and fragments are typically found in monoclonal antibody products as a result of degradation or modification. Important Quality Attributes identified for tremelimumab include the following listed below (Table 2).

TABLE 2

Aggregates
CDR Deamidation (Light Chain Asn-30)
Fragments
CDR Oxidation (Heavy Chain Trp-52)
Fe Oxidation (Met-256 and Met-432)

### Aggregation

Aggregation is one of the major degradation pathways of therapeutic antibodies. Multiple aggregation mechanisms have been observed in protein solutions, including association of native monomers, aggregation of conformationally altered monomers, aggregation of chemically modified monomers, nucleation-controlled aggregation, and surface-induced aggregation. Aggregates can be broadly divided into two classes, covalent and non-covalent. Non-covalent aggregates can be further classified as reversible and non-reversible.

Tremelimumab aggregates, including both dimer and higher-molecular weight (HMW) aggregates, were measured by high performance size exclusion chromatography (UPSEC).

### Characterization of Aggregates

To evaluate the impact on biological activity, tremelimumab aggregates were induced by a combination of low pH (2.5 to 3.0) and high temperature (45° C.) treatment with shear force (shaking). The stressed materials were fractionated by preparative SEC to generate enriched aggregates. The aggregate and monomer fractions were subsequently characterized by physicochemical and biological analysis (Table 4). Results indicate that surprisingly, aggregates have only a moderate impact on biological activity as decrease in relative potency was only seen with high aggregate fractions.

TABLE 4
Impact of Aggregates on Potency

| | Aggregate Level | Relative |
|---|---|---|
| Sample | by HPSEC (%) | Potency (%) |
| Stressed starting material | 25.9 | 91 |
| Prep SEC aggregate fraction | 77.4 | 73 |
| Prep SEC monomer fraction | 12.7 | 98 |

### SUMMARY

Results demonstrated that tremelimumab remained active at relatively high levels of aggregation.

Aggregation increased after materials were subject to stressed conditions i.e., low pH, high temperature and shear force. The impact of aggregates on biological activity is moderate. Reduced relative potency was observed when materials had >70% aggregates.

### CDR Deamidation (Light Chain Asn-30)

Asparagine deamidation is another degradation pathway of therapeutic antibodies. When deamidation occurs in the CDR (complementarity determining region), its impact on biological activity varies. The impact of deamidation on activity can vary from product to product. It has been reported that CDR deamidations at either Asn-33, Asn-55, or Asn-102 in IgGs has been found to decrease antigen binding and biological activity. Further, succinimide formation, a deamidation intermediate, at Asn-55 in the CDR2 region of an IgG1 has been reported to have reduced the biological activity by 70% and ligand binding affinity by 50%. Subsequent hydrolysis of this succinimide resulted in a further drop in potency. The impact of deamidation on protein activity is impossible to predict with the degree of certainty necessary for pharmaceutical compositions.

Tremelimumab (IgG2) has one CDR deamidation site at Asn-30 on the light chain. LC Asn-30 deamidation, as measured by peptide mapping, ranged from 6.5-6.9% for the samples tested (Table 5).

TABLE 5
Deamidation Measured by Peptide Mapping
and cIEF of Tremelimumab Samples

| Tremelimumab | Deamidation by Peptide | Total Acidic |
|---|---|---|
| Samples | Mapping LC Asn-30 (%) | Peaks by cIEF (%) |
| 1 | 6.5 | 18.8 |
| 2 | 6.5 | 17.5 |
| 3 | 6.9 | 18.7 |
| 4 | 6.9 | 17.9 |
| 5 | 6.8 | 17.7 |

### Characterization of CDR Deamidation (LC Asn-30)

When analyzing tremelimumab samples stressed at 40° C. during the stability study, a significant increase in deamidation at LC Asn-30 was observed by peptide mapping. These data are consistent with an increase in acidic peaks by cIEF (Table 6).

TABLE 6
Deamidation Measured by Peptide Mapping
and cIEF in 40° C. Heat-stressed Samples

| | LC Asn-30 Deamidation | Total Acidic |
|---|---|---|
| Time points (Month) | by Peptide Mapping (%) | Peaks by cIEF (%) |
| 1 | 18.3 | 31.0 |
| 2 | 29.6 | 43.1 |
| 3 | 37.7 | 51.9 |

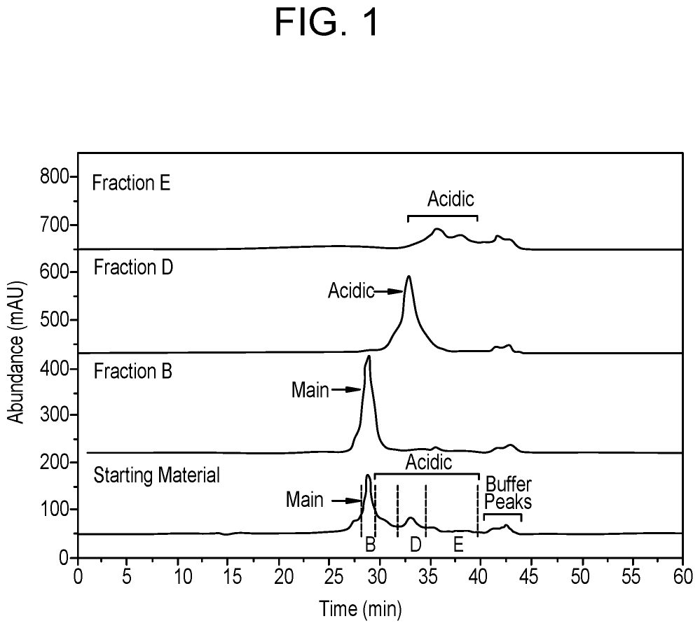

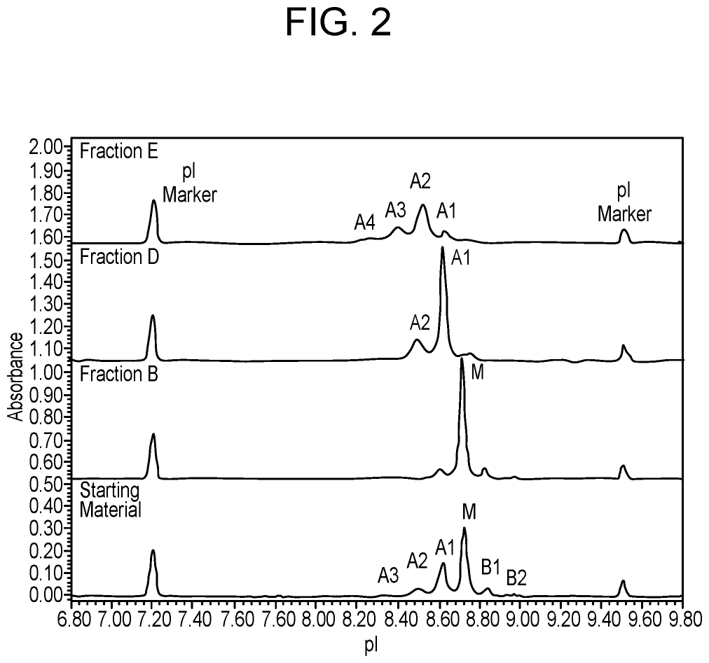

To evaluate the impact of deamidation on biological activity, tremelimumab was incubated at pH 9 for 7 days, then fractionated by ion exchange chromatography (IEC, FIG. 1) to enrich LC Asn-30 deamidated species. The main peak (M) fraction (Fraction B) together with two acidic fractions (Fraction D, Fraction E) containing LC Asn-30 deamidation were subsequently characterized by physicochemical and biological analyses. cIEF analyses of the IEC fractions are shown in FIG. 2.

Table 7 summarizes LC Asn-30 deamidation levels in the IEC fractions by peptide mapping and cIEF, as well as its impact on biological activity (reporter gene bioassay), CTLA-4 binding and FcRn binding activity. IEC fraction D contained 41.2% Asn-30 deamidation and eluted mainly as acidic peak 1 (A1) in cIEF. IEC fraction D showed reduced biological activity (78%) and decreased CTLA-4 binding (62%). The CTLA-4 binding was lower (54%) for IEC fraction E, which had higher LC Asn-30 deamidation and eluted mainly as acidic peak 2 (A2) in cIEF.

TABLE 7
Impact of Deamidation on Relative Potency, Relative
CTLA-4 Binding and Relative FcRn Binding Activity

| | Deamidation at | | | | | | | | |
|---|---|---|---|---|---|---|---|---|---|
| | LC.Asn- 30 by | | | | | | | Rel. | Rel. |
| | Peptide | | | | | | Rel. | CTLA-4 | FcRn |
| | Mapping | cIEF peaks (%) | | | | | Potency | Binding | Binding |
| IEC Fraction | (%) | A4 | A3 | A2 | A1 | M | (%) | (%) | (%) |
| B (main peak) | 2.3 | 0 | 0 | 0 | 10.5 | 80.6 | 103 | 97 | 104 |
| D (acidic peak 1) | 41.2 | 0 | 0 | 24.7 | 68.5 | 6.8 | 78 | 62 | 95 |
| E (acidic peak 2) | 53.8 | 9.4 | 23.6 | 50.4 | 11.4 | 5.2 | NT | 54 | 52 |

A1 = acidic peak 1;
A2 = acidic peak 2;
A3 = acidic peak 3;
A4 = acidic peak 4,
M = main peak;
NT = not tested

Further characterization was conducted following incubation of tremelimumab in PBS and in human serum at 37° C. to assess changes in deamidation level under physiological conditions. Deamidation at LC Asn-30 was the major degradation product detected in these studies (Table 8).

TABLE 8
LC Asn-30 Deamidation in Tremelimumab
Incubated with Human Serum and PBS

| | | | |
|---|---|---|---|
| Incubation | Deamidation of LC Asn-30 | Acidic Peaks | Relative |
| time (week) | by Peptide Mapping (%) | by cIEF (%) | Potency (%) |

Human Serum incubation study

| | | | |
|---|---|---|---|
| 0 | 3.4 | 29.1 | 106 |
| 1 | 11.1 | 46.0 | 98 |
| 2 | 22.9 | 65.8 | 91 |
| 3 | 33.9 | 80.0 | 82 |
| 4 | 34.8 | 76.9 | 76 |
| 5 | 37.7 | 76.5 | 80 |

PBS incubation study

| | | | |
|---|---|---|---|
| 0 | 2.2 | 26.2 | 92 |
| 1 | 8.8 | 41.2 | 96 |
| 2 | 18.5 | 56.2 | 83 |
| 3 | 27.0 | 66.0 | 93 |
| 4 | 37.7 | 75.7 | 76 |
| 5 | 44.7 | 81.3 | 76 |

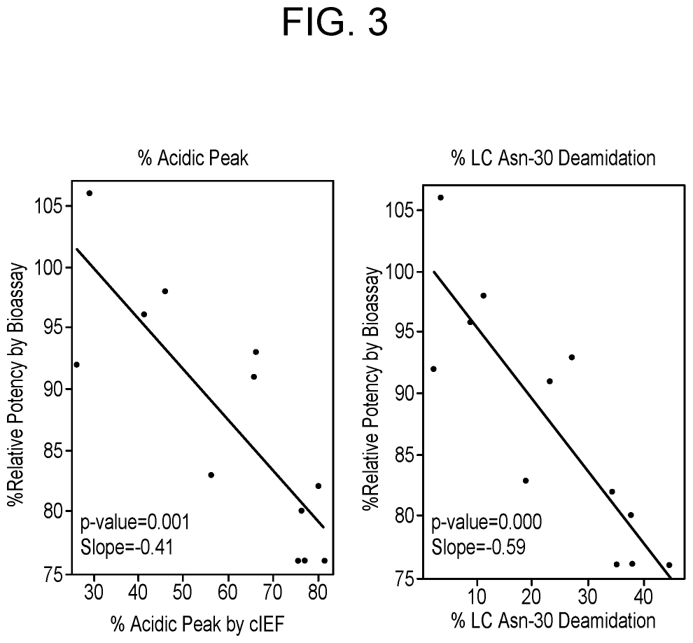

The correlations between biological activity and LC Asn-30 deamidation level (as measured by cIEF acidic peaks and by peptide mapping) are shown in FIG. 3. The impact of LC Asn-30 deamidation on biological activity is moderate. More than 75% relative potency was maintained when LC Asn-30 deamidation was >80% by cIEF acidic peaks, or >40% by peptide mapping.

### SUMMARY

Deamidation increased under 40° C. heat-stressed conditions (37.7% by peptide mapping after 3 months), and during serum or PBS incubation at 37° C. The impact of CDR deamidation at LC Asn-30 on biological activity is moderate. There was more than 75% relative potency remaining in materials with over 80% cIEF acidic peaks.

### Fragmentation

Fragmentation is a further degradation pathway of monoclonal antibodies. Fragmentation can occur at sites adjacent to Asp and His residues and has been shown to be influenced by the presence of metal ions, oxidative free radical impurities, or residual host cell proteolytic enzymes. The hinge region of an IgG2 is less prone to cleavage than IgG1. Total fragmentation of an IgG2 is generally less than that of an IgG1 under similar conditions.

### Characterization of Fragments

Tremelimumab is resistant to fragmentation, even under stressed conditions for an extended period of time. Fragments in tremelimumab stressed at 40° C. up to 6 months were less than or equal to 6.3% by non-reducing gel electrophoresis and less than or equal to 5.0% by reducing gel electrophoresis, as shown in Table 10. The low level of fragment is consistent with literature reports that IgG2 is less prone to fragmentation due to lack of peptide bond hydrolysis in its hinge region.

TABLE 10
Fragment determination in Heat Stressed Tremelimumab
Samples by Gel Electrophoresis

| | | | Fragments by | Fragments by |
|---|---|---|---|---|
| | Temper- | | Non-reducing gel | Reducing gel |
| Tremelimumab | ature | | electrophoresis | electrophoresis |
| Samples | (° C.) | Month | (%) | (%) |
| 6 | Control | 0 | 1.7 | 0.5 |
| | 40 | 2 | 2.6 | 0.6 |
| | | 3 | 2.7 | 1.7 |
| | | 6 | 5.9 | 5.0 |
| 7 | Control | 0 | 1.8 | 0.6 |
| | 40 | 2 | 2.6 | 0.5 |
| | | 3 | 3.4 | 2.2 |
| | | 6 | 6.1 | 4.2 |
| 8 | Control | 0 | 1.4 | 0.5 |
| | 40 | 2 | 2.2 | 0.6 |
| | | 3 | 3.0 | 2.4 |
| | | 6 | 6.3 | 4.8 |

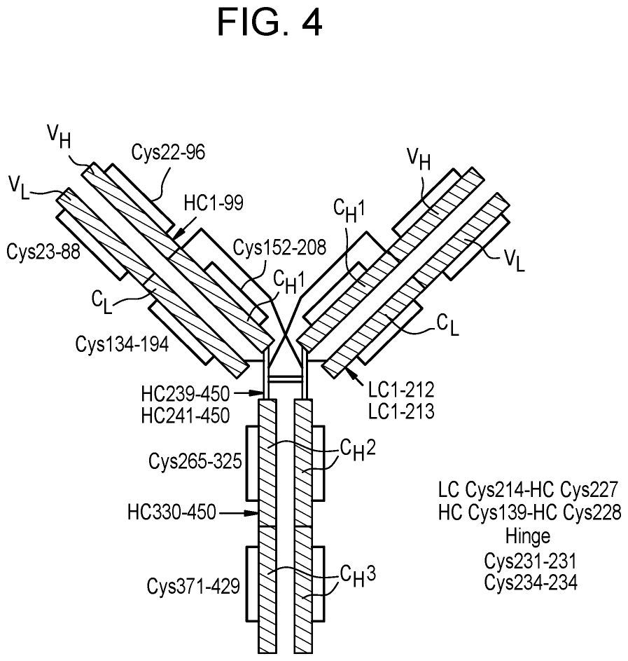

To identify the fragments observed by gel electrophoresis, control and heat stressed (40° C. for 6 months) samples were analyzed by RP-LC/MS. Compared to undetectable levels of fragment in the control sample, fragments were detected in the heat stressed sample. Major fragments were determined to be heavy chain fragments (HC 1-99, HC330-450), light chain fragments (LCl-212, LCl-213), and the light chain fragments with succinimide. Succinimide is a deamidation or isomerization intermediate. Succinimide intermediates are identified in heat stressed IgGs which have increased levels of deamidation or isomerization. Trace amounts of heavy chain fragments HC 239-450 and HC 241-450 were also detected. A schematic representation of tremelimumab with the fragmentation sites labelled is shown in FIG. 4.

The tremelimumab fragmentation pattern was fully characterized by orthogonal physicochemical methods as summarized in Table 11. Expected fragments and other product-related species detected in heat stressed tremelimumab by RP-LC/MS are listed. Gel electrophoresis peak identification was based on results from RP-LC/MS, deglycosylation study and the partial reduction study. In addition, peptide mapping was performed for verification of fragments and aglycosylated species.

TABLE 11
Tremelimumab Fragments by RP-LC/MS and Gel Electrophoresis

| Mass | Peak Identified by | NR Gel | R Gel |
|---|---|---|---|
| (Da) | RP-LC/MS | Electrophoresis | Electrophoresis |
| 13604 | HC C fragments (330-450) | HC C fragment | HC C fragment |
| 25234 | HC C fragments (241-450) | HC C fragment | HC C fragment |
| 25404 | HC C fragment (239-450) | | |
| 23150 | LC C fragment (1-212) | LC | LC |
| 23280 | LC C fragment (1-213) | LC | LC |
| 23133 | LC C fragment (1-212) | Su-LC | Su-LC |
| | with succinimide | | |
| 23263 | LC C fragment (1-213) | Su-LC | Su-LC |
| | with succinimide | | |
| 11019 | HC N fragment (1-99) | HC N fragment | HCN fragment |
| 148982 | IgG (G0f/G0t) | IgG | ND |

HC: heavy chain;
LC: light chain;
ND: not detected
NR: non-reducing;
R: reducing;
Su-LC: LC with succinimide

To evaluate the impact of fragmentation on biological activity, the relative potency (reporter gene assay) was determined for tremelimumab samples stressed at 40° C. for 0, 2 and 3 months (Table 12). Results demonstrate that >9500 relative potency was maintained after incubation for 3 months at 40° C. in the presence of low levels of fragments.

TABLE 12
Impact of Fragment on Potency

| Sample | Fragments by Non-reducing | Relative |
|---|---|---|
| Description | Gel Electrophoresis (%) | Potency (%) |
| 40° C. 0 Months | 1.7 | 95 |
| 40° C. 2 Months | 2.6 | 97 |
| 40° C. 3 Months | 2.7 | 97 |

### Summary

The fragmentation pattern was characterized by RP-LC/MS. Predominant fragment species were heavy chain N-terminal fragments (HC 1-99), heavy chain C-terminal fragments (HC 330-450), and light chain C-terminal fragments (LC 1-212 and LC 1-213).

Hinge region fragments were not detected. Tremelimumab fragmentation occurred very slowly under stressed conditions (40° C.). The presence of low level fragments (2.7% after 3 months at 40° C.) had little impact on biological activity. Fragmentation is not a major degradation pathway for tremelimumab.

### Oxidation

Amino acid oxidation is known to affect the structure, activity, and degradation rate of proteins. Met and Trp oxidation are chemical modifications that occur during monoclonal antibody purification, formulation, and storage. Oxidation of other residues (Cys and Tyr) has also been observed. Fe oxidation (Met-252 and Met-428; EU numbering) has been reported to decrease FcRn binding and cause shorter serum half-life. Biological activity loss has been shown to correlate with oxidation in the CDR region.

Tremelimumab oxidation was measured by tryptic peptide mapping as a characterization test at Trp-52 in the CDR, as well as at Met-256, Met-362, Met-401, and Met-432 in the Fe region. As the most susceptible site to oxidation, Met-256 oxidation was monitored by Lys-C peptide mapping.

### Characterization of Oxidation Heat Stress

Minor increases in oxidation of tryptophan and methionine residues were observed after thermal stress at 40° C. for 3 months (Table 14).

TABLE 14
Oxidation by Peptide Mapping of Heat-stressed Tremelimumab

| | Oxidation by Peptide Mapping (%) | | | | | |
|---|---|---|---|---|---|---|
| Time | HC Trp- | HC Met- | HC Met- | HC Met- | HC Met- | Relative |
| (month) | 52 | 256 | 362 | 401 | 432 | Potency (%) |
| 0 | 1.8 | 1.3 | 1.6 | 1.6 | 1.2 | 100 |
| 1 | 1.6 | 1.4 | 2.1 | 2.4 | 1.5 | 112 |
| 2 | 2.5 | 2.0 | 2.6 | 3.8 | 2.2 | 101 |
| 3 | 4.5 | 2.5 | 3.1 | 4.3 | 2.7 | 98 |

### Chemical Oxidation

Chemical oxidation was performed on tremelimumab samples by hydrogen peroxide treatment (100 ppm) at room temperature, prior to peptide mapping and biological analysis (Table 15). Met-256 and Met-401 oxidized more readily than Met-432 and Met-362. Met-256 oxidation up to 79.8% had no impact on potency. However, at oxidation levels above 35.5%, there was a reduction in FcRn binding.

TABLE 15
Effects of Chemical Oxidation on Potency and FcRn Binding

| | | | | | | | Relative |
|---|---|---|---|---|---|---|---|
| | | Oxidation by Peptide Mapping (%) | | | | Relative | FcRn |
| Incubation time | | HCMet- | HCMet- | HCMet- | HCMet- | Potency | Binding |
| with 100 ppm H₂O₂ | | 256 | 362 | 401 | 432 | (%) | (%) |
| Control t = 0 | | 1.2 | 0.9 | 1.4 | 0.2 | 100 | 89 |
| 8 | hours | 13.4 | 3.9 | 13.3 | 2.4 | 88 | 93 |
| 25 | hours | 35.5 | 9.7 | 28.7 | 7.8 | 83 | 91 |
| 2 | days | 55.8 | 17.8 | 44.6 | 20.7 | 88 | 37 |
| 3 | days | 68.5 | 26.5 | 55.2 | 42.1 | 83 | 28 |
| 4 | days | 79.8 | 36.3 | 63.7 | 53.0 | 98 | 24 |

### Photo-Oxidation

In addition to chemical oxidation, photo-stress studies were performed at the exposure level of IX and 3X as outlined by ICH guidance QIB; that is, optical radiation exposure exceeds 200 watt-hours per square meter for UV-A and 12 million lux-hours for cool white light (CWL). Another study was done on tremelimumab samples exposed to CWL intensity of approximately 2000 lux for 14 days (total 672000 lux hours).

Table 16 summarizes the level of oxidation in photo-stressed samples by peptide mapping, and its impact on potency and FcRn binding. No major impact on potency or FcRn binding was observed under exposure at IX ICH guideline or CWL 14 day exposure. Decrease in potency and FcRn binding occurred after exposures at 3X ICH guideline. Oxidation on Trp-52, Met-256, and Met-432 was observed at IX ICH guideline exposure, CWL 14 day exposure, and increased significantly after 3X ICH guideline exposures. Trp-52 is in the heavy chain CDR region and oxidation at this site could cause loss of potency under excess exposure of light (3X ICH). The study showed that photo-stressed oxidation of tryptophan in the CDR at 36.7% had an impact on biological activity.

TABLE 16
Oxidation, Potency, and FcRn Binding of Photo-stressed Tremelimumab Samples

| | | | | | | | Relative |
|---|---|---|---|---|---|---|---|
| | Oxidation by Peptide Mapping (%) | | | | | Relative | FcRn |
| Sample | HCTrp- | HCMet- | HCMet- | HCMet- | HCMet- | Potency | Binding |
| Description | 52 | 256 | 362 | 401 | 432 | (%) | (%) |

Photo-stress study according to ICH guideline

| | | | | | | | |
|---|---|---|---|---|---|---|---|
| Dark control | 3.3 | 2.8 | 1.3 | 1.3 | 0.6 | 107 | 91 |
| DP 1x ICH | 10.9 | 12.1 | 3.8 | 4.1 | 11.9 | 87 | 78 |
| DP 3x ICH | 36.7 | 41.9 | 12.8 | 15.8 | 34.6 | 59 | 41 |

Photo-stress study at 2000 tux per hour

| | | | | | | | |
|---|---|---|---|---|---|---|---|
| t = 0 control | 0.7 | 3.3 | 1.4 | 1.6 | 1.0 | 102 | 98 |
| DP (2R vial, | 6.4 | 7.8 | 2.5 | 2.7 | 5.4 | 97 | 92 |
| horizontal) 14 | | | | | | | |
| DP (2R vial | 5.4 | 7.1 | 2.2 | 2.5 | 4.7 | 90 | 94 |
| vertical) | | | | | | | |

### Summary

Chemical oxidation and photo-stress studies demonstrated that Met-256 oxidation up to 35.5% did not impact potency or FcRn binding. Trp-52 oxidation at 36.7% reduced potency and FcRn binding.

### Host Cell Protein (HCP)

LC-MS characterization of HCP was performed on tremelimumab and pre-purification samples. The results are summarized in the Tables 17-21. 20 or less HCPs were identified in tremelimumab samples, whereas 569-660 HCPs were detected in three different pre-purification samples (3-5), respectively. The method is able to achieve high sensitivity for HCP identification by using native digestion to deplete majority of mAb molecules. The concentration of each identified HCP was estimated by label-free quantitation approach. The results confirmed clearance of HCPs through purification process, though a few low abundant HCPs remained at reduced levels.

TABLE 17

| Tremelimumab | | | | | | |
|---|---|---|---|---|---|---|
| samples | 1 | 9 | 10 | 3 | 4 | 5 |
| Drug mg/mL | 19.6 | 20.1 | 19.9 | 10 | 10 | 10 |
| # HCPs Identified | 3 | 16 | 20 | 660 | 569 | 601 |
| HCP LC-MS ng/mg* | 2.4 | 8.0 | 13.0 | 2,700.0 | 1,400.0 | 2,400.0 |

*Semi-quantitative

TABLE 18

| | | | | | Sample | Sample | Sample |
|---|---|---|---|---|---|---|---|
| | Description | | MW | calc. | 1 | 9 | 10 |
| Accession | OS = Mus musculus | # AAs | [kDa] | pI | μg/mL | μg/mL | μg/mL |
| P10126 | Elongation factor 1-alpha 1 | 462 | 50.1 | 9.01 | | 2.15E−03 | 2.97E−03 |
| P19096 | Fatty acid synthase | 2504 | 272.3 | 6.58 | | | 1.38E−03 |
| P16858 | Glyceraldehyde-3-phosphate | 333 | 35.8 | 8.25 | | 1.29E−02 | 1.73E−02 |
| | dehydrogenase | | | | | | |
| Q9JKR6 | Hypoxia up-regulated protein 1 | 999 | 111.1 | 5.19 | | | 4.99E−03 |
| P20029 | Endoplasmic reticulum | 655 | 72.4 | 5.16 | 1.49E−02 | | 8.83E−02 |
| | chaperone BiP | | | | | | |
| Q9WTM5 | RuvB-like 2 OS = Mus musculus | 463 | 51.1 | 5.64 | | 6.07E−03 | 1.00E−02 |
| P35700 | Peroxiredoxin-1 | 199 | 22.2 | 8.12 | | 2.80E−03 | 3.52E−03 |
| P42932 | T-complex protein 1 subunit | 548 | 59.5 | 5.62 | | 1.48E−03 | 4.11E−03 |
| | theta | | | | | | |
| Q9D8N0 | Elongation factor 1-gamma | 437 | 50 | 6.74 | 1.43E−02 | 3.73E−02 | 4.15E−02 |
| P80317 | T-complex protein 1 subunit | 531 | 58 | 7.08 | | 4.74E−03 | 9.77E−03 |
| | zeta | | | | | | |
| P70168 | Importin subunit beta-1 | 876 | 97.1 | 4.78 | | 1.52E−02 | 2.04E−02 |
| P14869 | 60S acidic ribosomal protein PO | 317 | 34.2 | 6.25 | | 1.22E−02 | 7.41E−03 |
| P14206 | 40S ribosomal protein SA | 295 | 32.8 | 4.87 | | 7.39E−04 | 2.66E−03 |
| P80313 | T-complex protein 1 subunit eta | 544 | 59.6 | 7.84 | | 1.61E−02 | 2.14E−02 |
| Q99LF4 | RNA-splicing ligase RtcB | 505 | 55.2 | 7.23 | | 1.19E−02 | 1.58E−02 |
| | homolog | | | | | | |
| Q78PY7 | Staphylococcal nuclease | 910 | 102 | 7.43 | | | 5.08E−04 |
| | domain-containing protein 1 | | | | | | |
| P14685 | 26S proteasome non-ATPase | 530 | 60.7 | 8.44 | 1.84E−02 | | |
| | regulatory subunit 3 | | | | | | |
| Q9DBG5 | Perilipin-3 | 437 | 47.2 | 5.62 | | 2.09E−03 | 3.29E−03 |
| Q8R1Q8 | Cytoplasmic dynein 1 light | 523 | 56.6 | 6.42 | | | 6.22E−04 |
| | intermediate chain 1 | | | | | | |
| Q9WVA3 | Mitotic checkpoint protein | 326 | 36.9 | 6.84 | | 1.53E−02 | |
| | BUB3 | | | | | | |
| Q4VAH7 | HEPACAM family member 2 | 463 | 51.4 | 6.64 | | | 4.29E−03 |
| Q922Q4 | Pyrroline-5-carboxylate | 320 | 33.6 | 7.77 | | | 4.67E−03 |
| | reductase 2 | | | | | | |
| Q99J09 | Methylosome protein 50 | 342 | 36.9 | 5.27 | | 7.61E−03 | |
| Q8R010 | Aminoacyl tRNA synthase | 320 | 35.4 | 7.83 | | 9.21E−03 | |
| | complex-interacting | | | | | | |
| | multifunctional protein 2 | | | | | | |

TABLE 19

| | | | | Average + |
|---|---|---|---|---|
| | Description | Average | STDEV | 5 STEV |
| Accession | OS = Mus musculus | ppm | ppm | ppm |
| P20029 | Endoplasmic reticulum | 1.7 | 2.36 | 13.54 |
| | chaperone BiP | | | |
| Q9D8N0 | Elongation factor 1-gamma | 1.6 | 0.73 | 5.21 |
| P80313 | T-complex protein 1 subunit | 0.6 | 0.56 | 3.41 |
| | eta | | | |
| P70168 | Importin subunit beta-1 | 0.6 | 0.53 | 3.24 |
| P16858 | Glyceraldehyde-3-phosphate | 0.5 | 0.45 | 2.75 |
| | dehydrogenase | | | |
| Q99LF4 | RNA-splicing ligase RtcB | 0.5 | 0.41 | 2.52 |
| | homolog | | | |
| Q9WTM5 | RuvB-like 2 OS = Mus | 0.3 | 0.25 | 1.53 |
| | musculus | | | |
| P80317 | T-complex protein 1 subunit | 0.2 | 0.24 | 1.46 |
| | zeta | | | |

| | | | |
|---|---|---|---|
| TOTAL | 6.0 | 5.5 | 34. |

Ppm: parts per million of active substance, ng/mg protein

A useful upper bound using a tolerance interval using about five times the standard deviation of the data set, which yields an upper bound of about 34 ng/mg. The useful upper bound for host cell protein is well below the range for currently licensed monoclonal antibodies (≤100 ng/mg protein) (Champion K. et al. Bioprocess International; 2005; 3(8):52-7).

A useful upper bound on each HCP can also be established based on the same method. For instance, an upper bound for endoplasmic reticulum chaperone BliP is approximately 14 ng/mg.

TABLE 21
Quality Attribute Summary

| Quality Attribute | Useful Range |
|---|---|
| Aggregates | 0-70% |
| Fragments | 0-10.0% |
| Oxidation (CDR HC Trp-52) | 0-37% |
| Oxidation (Fc Met-256, Met-432) | 0-35% |
| (Methionine Oxidation)* | |
| Deamidation (CDR LC Asn-30) | 0-80% cIEF acidic peak |
| (Acidic Variants)* | 0-40% by peptide mapping |
| Host Cell Proteins | ≤34 ng/mg |

What is claimed is:

1: A composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; and one or more deamidated variants of the anti-CTLA-4 antibody, wherein the composition comprises ≤45%, ≤40%, ≤35%, ≤30%, ≤20%, ≤15%, ≤10%, ≤5%, ≤4%, or ≤3% of the deamidated variants.

2: The composition according to claim 1, wherein the amount of the deamidated variants of the anti-CTLA antibody is between the lower limit of detection of the method used to identify the deamidated variant and 45%, 40%, 35%, 30%, 20%, 15%, 10%, 5%, 4%, 3%, 2%, or 1%.

3: The composition according to either claim 1 or claim 2, wherein the amount of the deamidated variants of the anti-CTLA antibody is:
(i) selected from any one of the following ranges: 0.01-45%, 0.01-40%, 0.01-35%, 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-45%, 0.05-40%, 0.05-35%, 0.05-30%, 0.05-25%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-45%, 0.1-40%, 0.1-35%, 0.1-30%, 0.1-25%, 0.1-20%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-45%, 0.5-40%, 0.5-35%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 45%, about 40%, about 35%, about 30%, about 25%, about 20%, about 10%, about 5%, about 4%, about 3%, about 2%, or about 1%.

4: The composition according to claim 3, wherein the amount of the deamidated variants of the anti-CTLA antibody is 0.01-45%.

5: The composition according to any one of the preceding claims, wherein the deamidated variants of the anti-CTLA-4 antibody comprise a deamidated residue that is an aspartic acid residue, a succinimide-aspartic acid residue, or an iso-aspartic acid residue.

6: The composition according to any one of the preceding claims, wherein the deamidated residue or residues are within one or more CDRs of the anti-CTLA-4 antibody.

7: The composition according to any one of the preceding claims, wherein the deamidated residue or residues of the anti-CTLA antibody are within any one of the amino acid sequences of SEQ ID NOs: 1 to 6.

8: The composition according to any one of the preceding claims, wherein the deamidated variants of the anti-CTLA-4 antibody comprise deamidation at N30 in the CDRL1 of the anti-CTLA-4 antibody.

9: The composition according to any one of the preceding claims, wherein the deamidated variants of the anti-CTLA-4 antibody comprise deamidation at N30 of the amino acid sequence of SEQ ID NO: 1.

10: The composition according to any one of the preceding claims, wherein the deamidated variants of the anti-CTLA-4 antibody comprise the amino acid sequence of SEQ ID NO: 11.

11: The composition according to any one of the preceding claims, wherein the deamidated variants of the anti-CTLA-4 antibody comprise 0.1-45%, 0.1-35%, 0.1-30%, 0.1-25%, 0.1-20%, 0.1-10%, 0.1-5%, 0.1-1%, 1-45%, 1-35%, 1-30%, 1-25%, 1-20%, 1-10%, 1-5%, 5-45%, 5-35%, 5-30%, 5-25%, 5-20%, or 5-10% deamidation at N30 of the anti-CTLA-4 antibody.

12: The composition according to any one of the preceding claims, wherein the deamidated variants of the anti-CTLA-4 antibody comprise 1% or more, 2% or more, 3% or more, 4% or more, 5% or more, 6% or more, 7% or more, 8% or more, 9% or more, or 10% or more deamidation at N30 of the anti-CTLA-4 antibody.

13: The composition according to any one of the preceding claims, wherein the deamidated variants of the anti-CTLA-4 antibody comprise 0.1-45%, 0.1-35%, 0.1-30%, 0.1-25%, 0.1-20%, 0.1-10%, 0.1-5%, 0.1-1%, 1-45%, 1-35%, 1-30%, 1-25%, 1-20%, 1-10%, 1-5%, 5-45%, 5-35%, 5-30%, 5-25%, 5-20%, or 5-10% deamidation at N388 of the anti-CTLA-4 antibody.

14: The composition according to any one of the preceding claims, wherein the deamidated variants of the anti-CTLA-4 antibody comprise 1% or more, 2% or more, 3% or more, 4% or more, 5% or more, 6% or more, 7% or more, 8% or more, 9% or more, or 10% or more deamidation at N388 of the anti-CTLA-4 antibody.

15: The composition according to any one of the preceding claims, wherein the deamidated variants of the anti-CTLA-4 antibody comprise 0.1-45%, 0.1-35%, 0.1-30%, 0.1-25%, 0.1-20%, 0.1-10%, 0.1-5%, 0.1-1%, 1-45%, 1-35%, 1-30%, 1-25%, 1-20%, 1-10%, 1-5%, 5-45%, 5-35%, 5-30%, 5-25%, 5-20%, or 5-10% deamidation at N393 of the anti-CTLA-4 antibody.

16: The composition according to any one of the preceding claims, wherein the deamidated variants of the anti-CTLA-4 antibody comprise 1% or more, 2% or more, 3% or more, 4% or more, 5% or more, 6% or more, 7% or more, 8% or more, 9% or more, or 10% or more deamidation at N393 of the anti-CTLA-4 antibody.

17: The composition according to any one of the preceding claims, wherein the composition comprises:
(i) deamidation at N30 of the anti-CTLA-4 antibody;
(ii) deamidation at N388 of the anti-CTLA-4 antibody; and/or
(iii) deamidation at N393 of the anti-CTLA-4 antibody.

18: The composition according to any one of the preceding claims, wherein the composition comprises:
(i)≤45%, ≤40%, ≤35%, ≤30%, ≤25%, ≤20%, ≤16%, ≤15%, 12.5%, ≤10%, ≤5%, ≤5%, ≤4%, ≤3%, ≤2%, ≤1%, or ≤0.5 deamidation at N30 of the anti-CTLA-4 antibody;
(ii)≤45%, ≤40%, ≤35%, ≤30%, ≤25%, ≤20%, ≤16%, ≤15%, ≤12.5%, ≤10%, ≤5%, ≤5%, ≤4%, ≤3%, ≤2%, ≤1%, or ≤0.5 deamidation at N388 of the anti-CTLA-4 antibody; and/or
(iii)≤45%, ≤40%, ≤35%, ≤30%, ≤25%, ≤20%, ≤16%, ≤15%, ≤12.5%, ≤10%, ≤5%, ≤5%, ≤4%, ≤3%, ≤2%, ≤1%, or ≤0.5 deamidation at N393 of the anti-CTLA-4 antibody.

19: The composition according to any one of the preceding claims, wherein the anti-CTLA-4 antibody and the acidic variants of the anti-CTLA-4 antibody comprise a light chain variable domain having an amino acid sequence that is at least about 90% identical to the amino acid sequence of SEQ ID NO: 7 and a heavy chain variable domain having an amino acid sequence that is at least about 90% identical to the amino acid sequence of SEQ ID NO: 8.

20: The composition according to claim 19, wherein the anti-CTLA-4 antibody comprises a light chain variable domain having the amino acid sequence of SEQ ID NO: 7 and a heavy chain variable domain having the amino acid sequence of SEQ ID NO: 8.

21: The composition according to any one of the preceding claims, wherein the anti-CTLA-4 antibody and the acidic variants of the anti-CTLA-4 antibody comprise a light chain having an amino acid sequence that is at least about 90% identical to the amino acid sequence of SEQ ID NO: 9 and a heavy chain having an amino acid sequence that is at least about 90% identical to the amino acid sequence of SEQ ID NO: 10.

22: The composition according to claim 21, wherein the anti-CTLA-4 antibody comprises a light chain having the amino acid sequence of SEQ ID NO: 9 and a heavy chain having the amino acid sequence of SEQ ID NO: 10.

23: The composition according to claim 21, wherein the acidic variants of the anti-CTLA-4 antibody comprise deamidation at one or more of N388 and/or N393 of the amino acid sequence of SEQ ID NO: 10.

24: The composition according to any one of the preceding claims, wherein the deamidated variants of the anti-CTLA-4 antibody further comprise deamidation at N388 and/or N393 of the heavy chain of the anti-CTLA-4 antibody.

25: The composition according to any one of the proceeding claims, wherein the method used to identify the deamidated variant or deamidation at a residue of the anti-CTLA-4 antibody is capillary isoelectric focusing (cIEF).

26: The composition according to any one of the preceding claims, wherein the composition further comprises oxidized variants of the anti-CTLA-4 antibody.

27: A composition comprising an anti-CTLA antibody, wherein the anti-CTLA antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3; and a heavy chain variable domain comprising a CDRH1 having the amino acid sequence of SEQ ID NO: 4, a CDRH2 having the amino acid sequence of SEQ ID NO: 5, and a CDRH3 having an amino acid sequence of SEQ ID NO: 6; and one or more oxidized variants of the anti-CTLA antibody,
wherein the composition comprises ≤35%, ≤30%, ≤20%, ≤15%, ≤10%, ≤5%, ≤4%, or ≤3% of the oxidized variants.

28: The composition according to claim 27, wherein the amount of the oxidized variant of the anti-CTLA antibody is between the lower limit of detection of the method used to identify the oxidized variant and 35%, 30%, 20%, 15%, 10%, 5%, 4%, 3%, 2%, or 1%.

29: The composition according to either claim 27 or claim 28, wherein the amount of the oxidized variant of the anti-CTLA antibody is:
(i) selected from any one of the following ranges: 0.01-35%, 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-35%, 0.05-30%, 0.05-25%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-35%, 0.1-30%, 0.1-25%, 0.1-20%, 0.1-16%, 0.1-15%, 0.1-12.5%, 0.1-10%, 0.1-7.5%, 0.1-5%, 0.14%, 0.1-3%, 0.1-2%, 0.1-1%, 0.5-35%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 35%, about 30%, about 25%, about 20%, about 10%, about 5%, about 4%, about 3%, about 2%, or about 1%.

30: The composition according to claim 29, wherein the amount of the oxidized variant of the anti-CTLA antibody is 0.01-35%.

31: The composition according to any one of claims 24-30, wherein the oxidized residue or residues are within one or more CDRs of the anti-CTLA antibody.

32: The composition according to any one of claims 24-31, wherein the oxidized variants of the anti-CTLA antibody comprise oxidation at a tryptophan residue or a methionine residue.

33: The composition according to any one of claims 24-32, wherein the oxidized residue or residues of the anti-CTLA antibody are within any one of the amino acid sequences of SEQ ID NOs: 1 to 6.

34: The composition according to any one of claims 24-33, wherein the oxidized variants of the anti-CTLA antibody comprise oxidation at W52 of the CDRH2 of the anti-CTLA-4 antibody.

35: The composition according to claim 34, wherein the composition comprises ≤30%, ≤25%, ≤20%, ≤16%, ≤15%, ≤12.5%, ≤10%, ≤5%, ≤5%, ≤4%, ≤3%, ≤2%, ≤1% or 0.5 oxidation at W52 of the CDRH2 of the anti-CTLA antibody.

36: The composition according to claim 35, wherein the composition comprises oxidation at W52 of the CDRH2 of the anti-CTLA antibody in an amount that is between the lower limit of detection of the method used to identify oxidation at W52 of the CDRH2 and 30%, 25%, 20%, 16%, 15%, 12.5%, 10%, 7.5%, 5%, 4%, 3% 2%, or 1%.

37: The composition according to any one of claims 24-36, wherein the composition comprises oxidation at W52 of the CDRH2 of the anti-CTLA antibody in an amount:
(i) selected from any one of the following ranges: 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-30%, 0.05-25%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-30%, 0.1-25%, 0.1-20%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 20%, about 10%, about 5%, about 4%, about 3%, about 2%, or about 1%.

38: The composition according to any one of claims 24-37, wherein the oxidized variants of the anti-CTLA antibody comprise the amino acid sequence of SEQ ID NO: 12.

39: The composition according to any one of claims 24-38, wherein the oxidized variant of the anti-CTLA antibody comprises oxidation at one or more of M256, M362, M401, and/or M432 of the heavy chain of the anti-CTLA antibody.

40: The composition according to any one of claims 24-38, wherein the composition comprises:
(i)≤35%, ≤30%, ≤25%, ≤20%, ≤16%, ≤15%, ≤12.5%, ≤10%, ≤5%, ≤5%, ≤4%, ≤3%, ≤2%, ≤1%, or ≤0.5 oxidation at M256 of the heavy chain of the anti-CTLA antibody;
(ii)≤12.5%, ≤10%, ≤5%, ≤5%, ≤4%, ≤3%, ≤2%, ≤1%, or ≤0.5 oxidation at M362 of the heavy chain of the anti-CTLA antibody;
(iii)≤16%, ≤15%, ≤12.5%, ≤10%, 5%, ≤55%, ≤4%, ≤3%, ≤2%, ≤1%, or ≤0.5 oxidation at M401 of the heavy chain of the anti-CTLA antibody; and/or
(iv)≤35%, ≤30%, ≤25%, ≤20%, ≤16%, ≤15%, ≤12.5%, ≤10%, ≤5%, ≤5%, ≤4%, ≤3%, ≤2%, ≤1%, or ≤0.5 oxidation at M432 of the heavy chain of the anti-CTLA antibody.

41: The composition according to any one of claims 24-39, wherein the composition comprises oxidation at M256 of the heavy chain of the anti-CTLA antibody in an amount that is between the lower limit of detection of the method used to identify oxidation at M256 of the heavy chain of the anti-CTLA antibody and 35%, 30%, 25%, 20%, 16%, 15%, 12.5%, 10%, 7.5%, 5%, 4%, 3%, 2%, or 1%.

42: The composition according to any one of claims 24-41, wherein the composition comprises oxidation at M256 of the heavy chain of the anti-CTLA antibody in an amount:
(i) selected from any one of the following ranges: 0.01-35%, 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-35%, 0.05-30%, 0.05-25%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-35%, 0.1-30%, 0.1-25%, 0.1-20%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-35%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 35%, about 30%, about 25%, about 20%, about 10%, about 5%, about 4%, about 3%, about 2%, or about 1%.

43: The composition according to any one of claims 24-42, wherein the composition comprises oxidation at M401 of the heavy chain of the anti-CTLA antibody in an amount that is between the lower limit of detection of the method used to identify oxidation at M401 of the heavy chain of the anti-CTLA antibody and 16%, 15%, 12%, 0.5%, 10%, 7.5%, 5%, 4%, 3%, 2%, or 1%.

44: The composition according to any one of claims 24-43, wherein the composition comprises oxidation at M401 of the heavy chain of the anti-CTLA antibody in an amount:
(i) selected from any one of the following ranges: 0.01-16%, 0.01-15%, 0.01-12.5%, 0.01-10%, 0.01-7.5%, 0.01-5%, 0.01-4%, 0.01-3%, 0.01-2%, 0.01-1%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 16%, about 15%, about 10%, about 5%, about 4%, about 3%, about 2%, or about 1%.

45: The composition according to any one of claims 24-44, wherein the composition comprises oxidation at M362 of the heavy chain of the anti-CTLA antibody in an amount that is between the lower limit of detection of the method used to identify oxidation at M362 of the heavy chain of the anti-CTLA antibody and 12.5%, 10%, 7.5%, 5%, 4%, 3%, 2%, or 1%.

46: The composition according to any one of claims 24-45, wherein the composition comprises oxidation at M362 of the heavy chain of the anti-CTLA antibody in an amount:
(i) selected from any one of the following ranges: 0.01-12.5%, 0.01-10%, 0.01-7.5%, 0.01-5%, 0.01-4%, 0.01-3%, 0.01-2%, 0.01-1%, 0.05-12.5%, 0.05-10%, 0.05-7.5%, 0.05-5%, 0.05-4%, 0.05-3%, 0.05-2%, 0.05-1%, 0.1-12.5%, 0.1-10%, 0.1-7.5%, 0.1-5%, 0.1-4%, 0.1-3%, 0.1-2%, 0.1-1%, 0.5-12.5%, 0.5-10%, 0.5-7.5%, 0.5-5%, 0.5-4%, 0.5-3%, 0.5-2%, and 0.5-1%; or
(ii) about 10%, about 5%, about 4%, about 3%, about 2%, or about 1%.

47: The composition according to any one of claims 24-46, wherein the composition comprises oxidation at M432 of the heavy chain of the anti-CTLA antibody in an amount that is between the lower limit of detection of the method used to identify oxidation at M432 of the heavy chain of the anti-CTLA antibody and 35%, 30%, 25%, 20%, 16%, 15%, 12.5%, 10%, 7.5%, 5%, 4%, 3%, 2%, or 1%.

48: The composition according to any one of claims 24-47, wherein the composition comprises oxidation at M432 of the heavy chain of the anti-CTLA antibody in an amount:
(i) selected from any one of the following ranges: 0.01-35%, 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-35%, 0.05-30%, 0.05-25%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-35%, 0.1-30%, 0.1-25%, 0.1-20%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-35%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 35%, about 30%, about 25%, about 20%, about 10%, about 5%, about 4%, about 3%, about 2%, or about 1%.

49: The composition according to any one of claims 26-48, wherein the amount of oxidation at M256 of the oxidized variant of the anti-CTLA-4 antibody is between the lower limit of detection of the method used to identify oxidation at M256 of the oxidized variant of the anti-CTLA-4 antibody and 35%, 30%, 25%, 20%, 16%, 15%, 12.5%, 10%, 7.5%, 5%, 4%, 3%, 2%, or 1%.

50: The composition according to any one of claims 26-49, wherein the composition comprises oxidation at M256 of the oxidized variant of the anti-CTLA-4 antibody in an amount:
(i) selected from any one of the following ranges: 0.01-35%, 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-35%, 0.05-30%, 0.05-25%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-35%, 0.1-30%, 0.1-25%, 0.1-20%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-35%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 35%, about 30%, about 25%, about 20%, about 10%, about 5%, about 4%, about 3%, about 2%, or about 1%.

51: The composition according to any one of claims 26-50, wherein the amount of oxidation at M401 of the anti-CTLA-4 antibody is between the lower limit of detection of the method used to identify oxidation at M401 of the anti-CTLA-4 antibody and 16%, 15%, 12.5%, 10%, 7.5%, 5%, 4%, 3%, 2%, or 1%.

52: The composition according to any one of claims 26-51, wherein the composition comprises oxidation at M401 of the anti-CTLA-4 antibody in an amount:
(i) selected from any one of the following ranges: 0.01-16%, 0.01-15%, 0.01-12.5%, 0.01-10%, 0.01-7.5%, 0.01-5%, 0.01-4%, 0.01-3%, 0.01-2%, 0.01-1%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 16%, about 15%, about 10%, about 5%, about 4%, about 3%, about 2%, or about 1%.

53: The composition according to any one of claims 26-52, wherein the amount of oxidation at M362 of the anti-CTLA-4 antibody is between the lower limit of detection of the method used to identify oxidation at M362 of the anti-CTLA-4 antibody and 12.5%, 10%, 7.5%, 5%, 4%, 3%, 2%, or 1%.

54: The composition according to any one of claims 26-53, wherein the composition comprises oxidation at M362 of the anti-CTLA-4 antibody in an amount:
(i) selected from any one of the following ranges: 0.01-12.5%, 0.01-10%, 0.01-7.5%, 0.01-5%, 0.01-4%, 0.01-3%, 0.01-2%, 0.01-1%, 0.05-12.5%, 0.05-10%, 0.05-7.5%, 0.05-5%, 0.05-4%, 0.05-3%, 0.05-2%, 0.05-1%, 0.1-12.5%, 0.1-10%, 0.1-7.5%, 0.1-5%, 0.1-4%, 0.1-3%, 0.1-2%, 0.1-1%, 0.5-12.5%, 0.5-10%, 0.5-7.5%, 0.5-5%, 0.5-4%, 0.5-3%, 0.5-2%, and 0.5-1%; or
(ii) about 10%, about 5%, about 4%, about 3%, about 2%, or about 1%.

55: The composition according to any one of claims 26-54, wherein the amount of oxidation at M432 of the anti-CTLA-4 antibody is between the lower limit of detection of the method used to identify oxidation at M432 and 35%, 30%, 25%, 20%, 16%, 15%, 12.5%, 10%, 7.5%, 5%, 4%, 3%, 2%, or 1%.

56: The composition according to ant one of claims 26-55, wherein the composition comprises oxidation at M432 of the anti-CTLA-4 antibody in an amount:
(i) selected from any one of the following ranges: 0.01-35%, 0.01-30%, 0.01-25%, 0.01-20%, 0.01-16%, 0.01-15%, 0.01-12.5%, 0.01-10%, 0.01-7.5%, 0.01-5%, 0.01-4%, 0.01-3%, 0.01-2%, 0.01-1%, 0.05-35%, 0.05-30%, 0.05-25%, 0.05-20%, 0.05-16%, 0.05-15%, 0.05-12.5%, 0.05-10%, 0.05-7.5%, 0.05-5%, 0.05-4%, 0.05-3%, 0.05-2%, 0.05-1%, 0.1-35%, 0.1-30%, 0.1-25%, 0.1-20%, 0.1-16%, 0.1-15%, 0.1-12.5%, 0.1-10%, 0.1-7.5%, 0.1-5%, 0.1-4%, 0.1-3%, 0.1-2%, 0.1-1%, 0.5-35%, 0.5-30%, 0.5-25%, 0.5-20%, 0.5-16%, 0.5-15%, 0.5-12.5%, 0.5-10%, 0.5-7.5%, 0.5-5%, 0.5-4%, 0.5-3%, 0.5-2%, and 0.5-1%; or
(ii) about 35%, about 30%, about 25%, about 20%, about 10%, about 5%, about 4%, about 3%, about 2%, or about 1%.

57: The composition according to any one of claims 26-56, wherein the method used to identify the oxidized variant or oxidation at a residue of the anti-CTLA-4 antibody is capillary isoelectric focusing (cIEF).

58: A composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; and one or more aggregated variants of the anti-CTLA-4 antibody, wherein the composition comprises ≤26%, ≤25%, ≤20%, ≤10%, ≤5%, ≤4%, ≤3%, ≤2%, or ≤1% of the aggregated variants.

59: The composition according to claim 58, wherein the amount of aggregated variants of the anti-CTLA-4 antibody is between the lower limit of detection of the method used to identify the aggregated variant of the anti-CTLA-4 antibody and 26%, 25%, 20%, 10%, 5%, 4%, 3%, 2%, or 1%.

60: The composition according to either claim 58 or claim 59, wherein the composition comprises the aggregated variant of the anti-CTLA-4 antibody in an amount:
(i) selected from any one of the following ranges: 0.01-26%, 0.01-25%, 0.01-20%, 0.01-10%, 0.01-5%, 0.01-4%, 0.01-3%, 0.01-2%, 0.01-1%, 0.05-26%, 0.05-25%, 0.05-20%, 0.05-10%, 0.05-5%, 0.05-4%, 0.05-3%, 0.05-2%, 0.05-1%; 0.1-26%, 0.1-25%, 0.1-20%, 0.1-10%, 0.1-5%, 0.1-4%, 0.1-3%, 0.1-2%, 0.1-1%, 0.5-26%, 0.5-25%, 0.5-20%, 0.5-10%, 0.5-5%, 0.5-4%, 0.5-3%, 0.5-2%, and 0.5-1%; or
(ii) about 10%, about 5%, about 4%, about 3%, about 2%, or about 1%.

61: The composition according to any one of claims 58-60, wherein the method used to identify the aggregated variants of the anti-CTLA-4 antibody is size exclusion chromatography.

62: The composition according to claim 61, wherein the size exclusion chromatography is size exclusion high performance liquid chromatography (SE-HPLC).

63: A composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; and a fragmented variant of the CTLA-4 antibody, wherein the composition comprises ≤10%, ≤5%, ≤4%, ≤3%, ≤2%, or 1% fragmented variant.

64: The composition according to claim 63, wherein the amount of fragmented variant of the anti-CTLA-4 antibody is between the lower limit of detection of the method used to identify the aggregated variant of the anti-CTLA-4 antibody and 10%, 5%, 4%, 3%, 2%, or 1%.

65: The composition according to either claim 63 or claim 64, wherein the composition comprises the fragmented variant of the anti-CTLA-4 antibody in an amount:
(i) selected from any one of the following ranges: 0.01-10%, 0.01-5%, 0.01-4%, 0.01-3%, 0.01-2%, 0.01-1%, 0.05-10%, 0.05-5%, 0.05-4%, 0.05-3%, 0.05-2%, 0.05-1%, 0.1-10%, 0.1-5%, 0.1-4%, 0.1-3%, 0.1-2%, 0.1-1%, 0.5-10%, 0.5-5%, 0.5-4%, 0.5-3%, 0.5-2%, and 0.5-1%; or
(ii) about 10%, about 5%, about 4%, about 3%, about 2%, or about 1%.

66: The composition according to any one of claims 63-65, wherein the fragmented variant comprises heavy chain N-terminal fragments (HC 1-99), heavy chain C-terminal fragments (HC 330-450), and light chain C-terminal fragments (LC 1-212 and LC 1-213).

67: The composition according to any one of claims 63-66, wherein the anti-CTLA-4 antibody comprises a heavy chain having the amino acid sequence of SEQ ID NO: 10 and a light chain having the amino acid sequence of SEQ ID NO: 9.

68. The composition according to any one of claims 63-67, wherein the method used to identify the aggregated variant of the anti-CTLA-4 antibody is reducing or non-reducing capillary gel electrophoresis (CGE).

69: A composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; and up to 100% heavy chain N-terminal pyro-glutamate variant and/or up to 100% heavy chain C-terminal lysine cleaved variant of the CTLA-4 antibody.

70: A composition comprising an anti-CTLA antibody, wherein the anti-CTLA-4 antibody comprises a light chain variable domain comprising a CDRL1 having the amino acid sequence of SEQ ID NO: 1, a CDRL2 having the amino acid sequence of SEQ ID NO: 2, and a CDRL3 having the amino acid sequence of SEQ ID NO: 3, and a heavy chain variable domain comprising a CDRH1 having the amino acid sequence of SEQ ID NO: 4, a CDRH2 having the amino acid sequence of SEQ ID NO: 5, and a CDRH3 having the amino acid sequence of SEQ ID NO: 6; further comprising ≤34 ng/mg of host cell protein (HCP).

71: The composition according to claim 70, further comprising ≤34 ng/mg HCP.

72: The composition according to either claim 70 or claim 71, wherein the HCP is selected from the group consisting of Elongation factor 1-alpha 1, Glyceraldehyde-3-phosphate dehydrogenase, RuvB-like 2 OS=Mus musculus, Peroxiredoxin-1, T-complex protein 1 subunit theta, Elongation factor 1-gamma, T-complex protein 1 subunit zeta, Importin subunit beta-1, 60S acidic ribosomal protein P0, 40S ribosomal protein SA, T-complex protein 1 subunit eta, RNA-splicing ligase RtcB homolog, Perilipin-3, Mitotic checkpoint protein BUB3, Methylosome protein 50, Aminoacyl tRNA synthase complex-interacting multifunctional protein 2, Fatty acid synthase, Hypoxia up-regulated protein 1, Endoplasmic reticulum chaperone BiP, Staphylococcal nuclease domain-containing protein 1, Cytoplasmic dynein 1 light intermediate chain 1, HEPACAM family member 2, and Pyrroline-5-carboxylate reductase 2.

73: The composition according to claim 72 wherein the composition further comprises ≤13.5 ppm endoplasmic reticulum chaperone, BiP, ≤5.21 ppm Elongation factor 1-gamma, ≤3.41 ppm T-complex protein 1 subunit eta, ≤3.24 ppm Importin subunit beta-1, <2.75 ppm Glyceraldehyde-3-phosphate dehydrogenase, ≤2.52 ppm RNA-splicing ligase RtcB homolog, ≤1.53 ppm RuvB-like 2 OS=Mus musculus and ≤1.46 ppm T-complex protein 1 subunit zeta.

74: The composition according to any preceding claim, wherein the anti-CTLA-4 antibody comprises a light chain variable domain having the amino acid sequence of SEQ ID NO: 7 and a heavy chain variable domain having the amino acid sequence of SEQ ID NO: 8.

75: The composition according to any preceding claim, wherein the anti-CTLA-4 antibody comprises a light chain having the amino acid sequence of SEQ ID NO: 9 and a heavy chain having the amino acid sequence of SEQ ID NO: 10.

76: The composition according to any preceding claim, wherein the acidic variant of the anti-CTLA-4 antibody and/or the oxidized variant of the anti-CTLA-4 antibody comprises a light chain having an amino acid sequence that is at least about 90% identical to the amino acid sequence of SEQ ID NO: 9 and a heavy chain having an amino acid sequence that is at least about 90% identical to the amino acid sequence of SEQ ID NO: 15.

77: The composition according to claim 76, wherein the acidic variant of the anti-CTLA-4 antibody and/or the oxidized variant of the anti-CTLA-4 antibody comprises a light chain having the amino acid sequence of SEQ ID NO: 9 and a heavy chain having the amino acid sequence of SEQ ID NO: 15.

78: The composition according to 75, wherein the anti-CTLA-4 antibody comprises oxidation at one or more of M256, M362, and/or M432 of the amino acid sequence of SEQ ID NO: 10.

79: The composition according to claim 76 or claim 77, wherein the composition comprises:
(i)≤35%, ≤30%, ≤25%, ≤20%, ≤16%, ≤15%, ≤12.5%, ≤10%, ≤5%, ≤5%, ≤4%, ≤3%, ≤2%, ≤1%, or ≤0.5 oxidation at M256 of the amino acid sequence of SEQ ID NO: 10;
(ii)≤12.5%, ≤10%, ≤5%, ≤5%, ≤4%, ≤3%, ≤2%, ≤1%, or ≤0.5 oxidation at M362 of the amino acid sequence of SEQ ID NO: 10;
(iii)≤16%, ≤15%, ≤12.5%, ≤10%, ≤5%, ≤5%, ≤4%, ≤3%, <^{2%}, <1%, or ≤0.5 oxidation at M401 of the amino acid sequence of SEQ ID NO: 10; and/or
(iv)≤35%, ≤30%, ≤25%, ≤20%, ≤16%, ≤15%, ≤12.5%, ≤10%, ≤5%, ≤5%, ≤4%, ≤3%, ≤2%, ≤1%, or ≤0.5 oxidation at M432 of the amino acid sequence of SEQ ID NO: 10.

80: A composition comprising an anti-CTLA-4 antibody having a heavy chain comprising the amino acid sequence of SEQ ID NO: 10 and a light chain comprising the amino acid sequence of SEQ ID NO: 9, wherein the composition comprises:
(i)≤35%, ≤30%, ≤20%, ≤15%, ≤10%, ≤5%, ≤4%, or ≤3% oxidized variant of the anti-CTLA-4 antibody;
(ii)≤26%, ≤25%, ≤20%, ≤10%, ≤5%, ≤4%, ≤3%, ≤2%, or ≤1% aggregated variant of the anti-CTLA-4 antibody;
(iii)≤45%, ≤40%, ≤35%, 30%, ≤20%, ≤15%, 10%, 5%, ≤54%, or ≤3% deamidated variant of the anti-CTLA-4 antibody;
(iv)≤10%, ≤5%, ≤4%, ≤3%, ≤2%, or ≤1% fragmented variant of the anti-CTLA-4 antibody.

81: A composition comprising an anti-CTLA-4 antibody having a heavy chain comprising the amino acid sequence of SEQ ID NO: 10 and a light chain comprising the amino acid sequence of SEQ ID NO: 9, wherein the composition further comprises:
(i)≤30% of a variant oxidized at Heavy Chain Trp-52 of the anti-CTLA-4 antibody;
(ii)≤35% of a variant deamidated at Heavy Chain Met256 and/or Heavy Chain Met-432 of the anti-CTLA-4 antibody;
(iii)≤34 ng/mg Host Cell protein;
(iv)≤4% aggregated variant of the anti-CTLA-4 antibody; and/or
(iv)≤10% fragmented variant of the anti-CTLA-4 antibody.

82: The composition according to any one of the preceding claims, wherein the anti-CTLA-4 antibody is a full-length antibody.

83: The composition according to any one of the preceding claims, wherein the anti-CTLA-4 antibody is human.

84: The composition according to any one of the preceding claims, wherein the composition is formed during the manufacture or storage of the antibody.

85: A pharmaceutical composition comprising the composition according to any one of the preceding claims and at least one pharmaceutically acceptable excipient.

86: A formulation comprising the pharmaceutical composition according to claim 84, wherein the formulation comprises about 5 mg/mL to about 125 mg/mL of anti-CTLA-4 antibody and a buffering agent at a pH of about 5.0 to about 6.5.

87: The formulation according to claim 86, wherein the buffering agent is histidine buffer.

88: The formulation according to claim 87, wherein the buffering agent is histidine buffer at a pH of about 5.5.

89: The formulation according to any one of claims 86-88, which additionally comprises trehalose dihydrate.

90: The formulation according to any one of claims 86-89, which additionally comprises polysorbate 80.

91: The formulation according to any one of claims 86-90, which additionally comprises a chelating agent.

92: A formulation comprising the pharmaceutical composition according to claim 85, wherein the formulation comprises:
(a) about 20 mg/mL of anti-CTLA-4 antibody;
(b) about 20 mM histidine buffer;
(c) about 222 mM trehalose dihydrate;
(d) about 0.27 mM disodium edetate dihydrate; and
(e) about 0.02% w/v polysorbate 80, at a pH of about 5.5.

93: An injection device comprising the composition according to any one of claims 1-84, the pharmaceutical composition according to claim 85, or the formulation according to any one of claims 86-92.

94: A cell culture medium comprising the composition according to any one of claims 1-84.

95: An eluate comprising the composition according to any one of claims 1-84.

96: A method of treating cancer comprising administering to a subject in need thereof a therapeutically effective amount of the composition according to any one of claims 1-84, the pharmaceutical composition according to claim 85, or the formulation according to any one of claims 86-92.

97: The method according to claim 96, wherein the composition is administered at a dose of from 50 mg to 400 mg, for example, 75 mg or 300 mg.

98: A composition according to any one of claims 1-84, the pharmaceutical composition according to claim 85, or the formulation according to any one of claims 86-92 for use in therapy.

99: A composition according to any one of claims 1-84, the pharmaceutical composition according to claim 85, or the formulation according to any one of claims 86-92 for use in the treatment of cancer.

100: Use of a composition according to any one of claims 1-84, the pharmaceutical composition according to claim 85, or the formulation according to any one of claims 86-92 in the manufacture of a medicament for use in the treatment of cancer.


SEQUENCE LISTING (15 sequences)

SEQ ID NO: 1 - protein, 11 aa, synthetic construct
RASQSINSYLD

SEQ ID NO: 2 - protein, 7 aa, synthetic construct
AASSLQS

SEQ ID NO: 3 - protein, 9 aa, synthetic construct
QQYYSTPFT

SEQ ID NO: 4 - protein, 10 aa, synthetic construct
GFTFSSYGMH

SEQ ID NO: 5 - protein, 15 aa, synthetic construct
VIWYDGSNKYYADSV

SEQ ID NO: 6 - protein, 16 aa, synthetic construct
DPRGATLYYYYYGMDV

SEQ ID NO: 7 - protein, 139 aa, synthetic construct
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 8 - protein, 167 aa, synthetic construct
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 9 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 11 aa, synthetic construct
RASQSIDSYLD

SEQ ID NO: 12 - protein, 15 aa, synthetic construct
  VARIANT 3: note = X is oxidized tryptophan
VIXYDGSNKYYADSV

SEQ ID NO: 13 - protein, 139 aa, synthetic construct
PSSLSASVGDRVTITCRASQSIDSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 14 - protein, 167 aa, synthetic construct
  VARIANT 42: note = X is oxidized methionine
GVVQPGRSLRLSCAASGFTFSSYGMHWVRQAPGKGLEWVAVIXYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 15 - protein, 450 aa, synthetic construct
  VARIANT 52: note = X is tryptophan or oxidized tryptophan
  VARIANT 256: note = X is methionine or oxidized methionine
  VARIANT 362: note = X is methionine or oxidized methionine
  VARIANT 401: note = X is methionine or oxidized methionine
  VARIANT 432: note = X is methionine or oxidized methionine
  VARIANT 388: note = X is asparagine which may be deamidated
  VARIANT 393: note = X is asparagine which may be deamidated
VQLVESGGGVVQPGRSLRLSCAASGFTFSSYGMHWVRQAPGKGLEWVAVIXYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLXISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEXTKNQVSLTCLVKGFYPSDIAVEWESXGQPEXNYKTTPPXLDSDGSFFLYSKLTVDKSRWQQGNVFSCSVXHEALHNHYTQKSLSLSPGK